(19) United States
(12) Patent Application Publication
(10) Pub. No.: US 2019/0358184 A1
(43) Pub. Date: Nov. 28, 2019

(54) Methods of treating amyloid-beta peptide diseases

(71) Applicant: Ecole Polytechnique Federale de Lausanne, Lausanne (CH)

(72) Inventors: Johan AUWERX, Lausanne (CH); Vincenzo SORRENTINO, Lausanne (CH); Laurent MOUCHIROUD, Lausanne (CH)

(21) Appl. No.: 16/468,791

(22) PCT Filed: Dec. 13, 2017
(86) PCT No.: PCT/EP2017/082584

§ 371 Date: Jun. 12, 2019

Related U.S. Application Data
(60) Provisional application No. 62/595,417, filed on Dec. 6, 2017.
(60) Provisional application No. 62/433,616, filed on Dec. 13, 2016.

(51) Int. Cl. A61K 31/201; A61K 31/65; A61K 31/225; A61K 31/352; A61K 31/706

(52) U.S. Cl. CPC A61K 31/201; A61K 31/65; A61K 31/706; A61K 31/352; A61K 31/225

(57) ABSTRACT

The present invention is directed to a method of treating an amyloid-3 peptide disease in a subject in need thereof, the method comprising administering to the subject a therapeutically effective amount of a compound that enhances mitochondrial proteostasis.

### RELATED APPLICATIONS

This application claims priority to, and the benefit of U.S. Provisional Application No. 62/433,616, filed Dec. 13, 2016 and U.S. Provisional Application No. 62/595,417, filed Dec. 6, 2017, both of which are incorporated herein by reference in their entireties.

### FIELD OF THE INVENTION

The present invention relates to methods of treating amyloid-β peptide diseases.

### BACKGROUND OF THE INVENTION

Aging is often accompanied by the onset of proteotoxic degenerative diseases, characterized by the accumulation of unfolded and aggregated proteins. Amyloid diseases are a subclass of proteotoxic disorders, which can affect the nervous system, like in the case of Alzheimer's (AD), the most common form of dementia, but also other organs, as exemplified by type-2 diabetes, amyloidosis-associated kidney disease, and inclusion body myositis (IBM), an aging-related muscle degeneration disease.

To date, no efficient therapy is available to treat or delay AD or IBM, two diseases with a strong component of amyloid-β (Aβ) aggregation. Clinical trials for AD have focused primarily on counteracting Aβ aggregation in the brain, considered the key pathogenic mechanism. However, increasing evidence suggests that AD is a complex multifactorial disease and mitochondrial dysfunction has emerged as a common pathological hallmark. Similarly, mitochondrial dysfunction has been identified as a typical feature of IBM. Mitochondrial abnormalities in both AD and IBM include decreased mitochondrial respiration and activity and alterations in mitochondrial morphology; however, the relevance of other key aspects of mitochondrial homeostasis, such as mitochondrial proteostasis, to these diseases is still largely unknown.

### SUMMARY OF THE INVENTION

One aspect of the present disclosure relates to a method of treating an amyloid-3 peptide disease in a subject in need thereof, the method comprising administering to the subject a therapeutically effective amount of a compound that enhances mitochondrial proteostasis.

In some embodiments, the compound can do the following: (a) induce mitochondrial unfolded protein response (UPR^{mt}), (b) induce mitochondrial biogenesis, (c) induce mitophagy, (d) modulate lipid metabolism, or a combination thereof.

In some embodiments, the compound that induces UPR^{mt} is selected from the group consisting of tetracycline, chlortetracycline, oxytetracycline, demeclocycline, lymecycline, meclocycline, methacycline, minocycline, rolitetracycline, doxycycline, tigecycline, actinonin, chloramphenicol, and a compound that blocks mitochondrial import.

In some embodiments, doxycycline is administered at 90 mg/kg/day in food for 9-10 weeks or 200-500 mg/kg/day in water for about 9-10 weeks.

In some embodiments, the compound that induces mitochondrial biogenesis is selected from the group consisting of an NAD⁺ boosting compound, a PARP inhibitor, a CD38 or CD157/BST1 inhibitor, an activator of nicotinamide phosphoribosyltransferases (NAMPT), an inhibitor of nicotinamide N methyltransferases (NNMT), and an inhibitor of α-amino-β-carboxymuconate-ε-semialdehyde decarboxylase (ACMSD).

In some embodiments, the NAD⁺ boosting compound is an NAD⁺ precursor, e.g., nicotinamide riboside, nicotinamide mononucleotide, nicotinic acid, or nicotinamide. Nicotinamide riboside can be administered at 400 mg/kg/day in food for about 9-10 weeks.

In some embodiments, the PARP inhibitor is 3-aminobenzamide, olaparib, velaparib, rucaparib, iniparib, talazoparib, CEP-9722, E7016, or niraparib. Olaparib or velaparib can be administered at 300 mg/kg/day in food for about 9-10 weeks.

In some embodiments, the CD38 or CD157/BST1 inhibitor is GSK 897-78c, apigenin, or quercetin.

In some embodiments, the activator of NAMPT is PC73.

In some embodiments, the inhibitor of ACMSD is a phthalate ester or pyrazinamide.

In some embodiments, the compound that induces mitophagy is selected from the group consisting of Urolithin A, Urolithin B, 5-aminoimidazole-4-carboxamide-ribonucleoside, salicylate, A-769662, 1-(2,6-Dichlorophenyl)-6-[[4-(2-hydroxyethoxy)phenyl]methyl]-3-propan-2-yl-2H-pyrazolo[3,4-d]pyrimidin-4-one, and metformin.

In some embodiments, the compound that modulates lipid metabolism is selected from the group consisting of perhexiline, a fibrate (e.g., fenofibrate, clofibrate, or bezafibrate), and a statin (e.g., lovastatin, simvastatin, atorvastatin, or fluvastatin).

In some embodiments, the compound that modulates lipid metabolism also inhibits sphingosin and ceramide synthesis.

In some embodiments, the compound that modulates lipid metabolism is myriocin, fumonisin B1, amlodipine, astemizole, benztropine, bepridil, or doxepine. Myriocin can be administered at 0.4 mg/kg body weight.

In some embodiments, the compound is a natural product, such as Urolithin A, nicotinamide riboside, nicotinamide mononucleotide, nicotinic acid, nicotinamide, and quercetin.

In some embodiments, the amyloid-β peptide disease is a muscle disease, such as inclusion body myositis, age-related sarcopenia, frailty of the elderly, and muscular dystrophy (e.g., Duchenne's or Becker's muscular dystrophy).

In some embodiments, the amyloid-β peptide disease is a metabolic disease, such as type 2 diabetes, an amyloid kidney disease, and an amyloid heart disease.

In some embodiments, the amyloid-β peptide disease is Alzheimer's disease, Dementia, Parkinson's disease, Huntington's disease, or amyotropic lateral sclerosis.

In some embodiments, the subject is a human.

In some embodiments, the method comprises administering to the subject a therapeutically effective amount of at least two compounds, each of which enhances mitochondrial proteostasis. The at least two compounds are administered sequentially or simultaneously.

### BRIEF DESCRIPTION OF THE DRAWINGS

FIGS. 1A-1H are graphs showing that mitochondrial function is perturbed in AD and results in the induction of a conserved mitochondrial stress response signature. (FIG. 1A) GSEA of genes involved in Oxphos and mitochondrial protein import in human Alzheimer prefrontal cortex expression dataset (GN328). (FIG. 1B) Heatmaps of genes involved in Oxphos and mitochondrial protein import obtained from the GSEA of GN328. Low expression is shown in blue, while high expression is in red. (FIG. 1C) Correlation plots of mitochondrial stress genes, UPR^{er} and HSR levels in human prefrontal cortex from AD patients (GN328). For further information, see FIGS. 6A-6G. (FIG. 1D) Transcript analysis of the Mitochondrial Stress Response signature (MSR) in brain tissues from patients with different stages of AD, i.e. no cognitive impairment (NCI, n=8), mild-cognitive impairment (MCI, n=8) and mild/moderate AD (n=8). (FIG. 1E) Western blot and quantification of mtDNaJ and CLPP expression in brain tissues of NCI, MCI and AD patients (NCI, MCI and AD, n=8). Data are representative of at least two independent experiments. (FIG. 1F) Transcript analysis of the MSR in cortex tissues of wild type (WT) and 3×TgAD mice at 9 months (WT, n=5; 3×TgAD, n=5) of age. (FIG. 1G) Immunoblot analysis (WT, n=6; 3×TgAD, n=6,) of selected mitochondrial proteostasis (LONP1) and autophagy (PINK, phospho-p62 and LC3) effectors in cortex tissues of 9 months WT and 3×TgAD mice. (FIG. 1H) Immunoblot analysis (WT, n=5; 3×TgAD, n=5,) of mitophagy and autophagy proteins in mitochondrial extracts from cortex tissues of 9 months WT and 3×TgAD mice. Values are mean±s.e.m. *P<0.05; **P≤0.01; ***P≤0.001; n.s. not significant. See Methods for details of the statistical test used. Mito., mitochondrial. For further information, see FIGS. 7A-7G.

FIGS. 2A-2J are graphs showing mitochondrial dysfunction and reliance on atfs-1 for survival and fitness of GMC101 worms upon proteotoxic stress. (FIG. 2A) Transcript analysis of the MSR of CL2122 and GMC101 worms at day 1 of adulthood (n=3). (FIG. 2B) Respiration at basal level and after 45 min incubation with FCCP (10 μM) at day 1 (Dl) and 3 (D3) of adulthood in CL2122 and GMC101 (n=10). (FIG. 2C) Immunoblot analysis (CL2122, n=5; GMC101, n=5, WB of 4 representative biological replicates) of OXPHOS proteins in control and GMC101 at day 1 of adulthood. (FIG. 2D) mtDNA/nDNA ratio at day 1 of adulthood in CL2122 and GMC101 (n=13 per group). (FIG. 2E) Representative images of CL2122 and GMC101 fed with atfs-1 RNAi after 4 days of development (n=2), and fraction of worms fed with atfs-1 RNAi at the larval, L4 and adult stage after 4 days (n=2). (FIG. 2F) Respiration at basal level and after 45 min incubation with FCCP (10 μM) at day 1 of adulthood in GMC101 fed with atfs-1 RNAi (n=6-10). (FIG. 2G) Transcript analysis of the MSR of GMC101 fed with atfs-1 RNAi at day 1 of adulthood (n=3). (FIG. 2H) Mobility of GMC101 fed with 50% dilution of atfs-1 RNAi from day 1 to 4 of adulthood (ev, n=33 to 59; atfs-1^{1/2}, n=39 to 47). (FIG. 2I) Amyloid aggregation in GMC101 upon atfs-1 RNAi shown by western-blotting of 2 biological repeats at day 1 of adulthood. Data are representative of at least two independent experiments. (FIG. 2J) Mobility of control and afts-1 overexpressing (AUW9 and AUW10) GMC101 worms from day 1 to 4 of adulthood (GMC101, n=41 to 57; AUW9, n=36 to 40; AUW10, n=36 to 38). Values are mean±s.e.m. *P<0.05; **P≤0.01; ***P≤0.001; ****P≤0.0001; n.s. not significant. See Methods for details of the statistical test used. ev, scrambled RNAi; A.U., arbitrary units. For further information, see FIGS. 8A-80 and 9A-9L.

FIGS. 3A-3K are graphs showing enhancing mitochondrial proteostasis by inhibiting mitochondrial translation increases fitness and reduces Aβ aggregation in GMC101 worms and in amyloid-expressing cells. (FIGS. 3A-3B) Transcript analysis of the MSR of GMC101 worms fed with mrps-5 RNAi (FIG. 3A, n=3) or treated with dox (FIG. 3B, n=3) at day 1 of adulthood. (FIG. 3C) Mobility of GMC101 worms fed with mrps-5 RNAi or treated with dox in control RNAi condition from day 1 to 6 of adulthood (n=20 to 54). (FIG. 3D) Percentage of paralyzed GMC101 worms after mrps-5 RNAi or dox at day 8 of adulthood (n=3). (FIG. 3E) Percentage of dead GMC101 after mrps-5 RNAi or dox at day 8 of adulthood (n=3). (FIG. 3F) Western-blot of amyloid aggregation in GMC101 fed with mrps-5 RNAi or treated with dox (15 μg/mL) at day 1 of adulthood (n=3). (FIG. 3G) Mobility of GMC101 fed with mrps-5 RNAi or treated with dox upon atfs-1 RNAi feeding from day 1 to 6 of adulthood (n=20 to 38). (FIG. 3H) Western-blot of amyloid aggregation in GMC101 worms treated with dox (15 μg/mL) upon atfs-1 RNAi feeding (n=3, WB representative of 2 biological replicates) at day 1 of adulthood. (FIG. 3I) Mobility of GMC101 fed with mrps-5 RNAi or treated with dox upon dct-1 RNAi feeding from day 1 to day 4 of adulthood (dox, dct-1, n=34 to 54; mrps-5, dct-1, n=41 to 45). Values are mean±s.e.m. *P<0.05; **P≤0.01; ***P≤0.001; ****P≤0.0001. (FIG. 3J) Western-blot of amyloid aggregation in GMC101 worms treated with dox (15 μg/mL) upon dct-1 RNAi feeding (n=3) at day 1 of adulthood. (FIG. 3K) Confocal images of the SH-SY5Y neuroblastoma cell line expressing the APP Swedish K670N/M671L double mutation (APP_{Swe}) stained with the anti-β-Amyloid 1-42, after treatment with dox (10 μg/mL) and, where indicated, ISRIB (0.5 μM) for 24 h. Scale bar, 10 μm. Data are representative of at least two independent experiments. ev, scrambled RNAi; dox., doxycycline; A.U., arbitrary units; ISRIB, integrated stress response inhibitor. For further information, see FIGS. 10A-10O.

FIGS. 4A-4M are graphs showing that supplementation with NAD⁺ boosters increases healthspan and reduces protein aggregation in GMC101 worms and in amyloid-expressing cells. Transcript analysis of the MSR of GMC101 worms treated with nicotinamide riboside (FIG. 4A, NR, n=3) or Olaparib (FIG. 4B, AZD, n=3) at day 1 of adulthood. (FIG. 4C) Mobility of GMC101 treated with NR (n=50) or AZD (n=39) from day 1 to 4 of adulthood. (FIG. 4D) Percentage of paralyzed GMC101 after treatment with NR (n=3) or AZD (n=3) at day 8 of adulthood. (FIG. 4E) Percentage of dead GMC101 worms after NR (n=3) or AZD (n=3) treatment at day 8 of adulthood. (FIG. 4F) Western-blot of amyloid aggregation in GMC101 after treatment with NR (n=3) or AZD (n=3) at day 1 of adulthood. Data are representative of at least two independent experiments. (FIG. 4G) Mobility of GMC101 treated with NR upon atfs-1 RNAi feeding from day 1 to 4 of adulthood (ev, n=31 to 52; atfs-1, n=36 to 38; NR, n=32 to 40; NR, atfs-1, n=36 to 41). (FIG. 4H) Percentage of paralyzed and dead GMC101 treated with NR upon atfs-1 RNAi feeding at day 8 of adulthood (n=3). (FIG. 4I) Western-blot of amyloid aggregation in GMC101 treated with NR upon atfs-1 RNAi feeding (n=3, WB representative of 2 biological replicates) at day 1 of adulthood. (FIG. 4J) Mobility of GMC101 worms treated with NR upon dct-1 RNAi feeding from day 1 to 4 of adulthood (ev, n=32 to 41; dct-1, n=38 to 40; NR, n=37 to 39; NR, dct-1, n=32 to 50). (FIG. 4K) Percentage of paralyzed and dead GMC101 worms treated with NR upon dct-1 RNAi feeding at day 8 of adulthood (n=3). Values are mean±s.e.m. *P<0.05; **P≤0.01; ***P≤0.001; ****P≤0.0001. (FIG. 4L) Immunoblot of amyloid aggregation in GMC101 worms treated with NR upon dct-1 RNAi feeding (n=3) at day 1 of adulthood. (FIG. 4M) Confocal images of the APP_{Swe} SH-SY5Y neuroblastoma cells stained with the anti-3-Amyloid 1-42, after treatment with NR (3 mM) for 24 h. Data are representative of at least two independent experiments. ev, scrambled RNAi; NR, nicotinamide riboside; A.U., arbitrary units. For further information, see FIGS. 11A-11L.

FIGS. 5A-5F are graphs showing that NR treatment reduces amyloid aggregation in aged mice and Aβ deposits in transgenic AD mice. (FIG. 5A) Representative images and corresponding quantification (relative % area) of amyloid immunostaining of TA muscle of young and old C57BL/6J mice with or without NR treatment using the anti-oligomer antibody A11 (n=5 per group). Scale bar, 50 m. (FIG. 5B) Representative images and corresponding quantification (relative % area) of amyloid immunostaining of TA muscle of young and old mice with or without NR using the anti-Aβ antibody 4G8 (n=5-7 per group). Scale bar, 50 m. (FIG. 5C) Immunoblot of the Oxphos components SDHB and MTCO1 and amyloid proteins with the 4G8 antibody from forelimbs muscles of young and old C57BL/6J mice with or without NR treatment (n=3 per group). (FIG. 5D) Representative images and corresponding quantification of plaque (relative % area and number) in cortex samples of APP/PSEN1 AD mice with or without NR treatment, using Thioflavin S (ThS) (n=5-7 per group; NR 400 mg/kg/day for 10 weeks). Scale bar, 200 m. (FIG. 5E) Transcript analysis of the MSR from cortex samples of APP/PSEN1 mice with or without NR treatment (n=5 per group). (FIG. 5F) Immunoblot of Oxphos proteins from cortex samples of APP/PSEN1 mice following NR treatment (n=4 per group). Values are mean±s.e.m. *P<0.05; **P≤0.01; ***P≤0.001. Values are mean±s.e.m. *P<0.05; **P≤0.01; ***P≤0.001. CD, chow diet. For further information, see FIGS. 12A-12G.

FIGS. 6A-6G are graphs showing that mitochondrial function pathways are perturbed in AD and IBM. In FIGS. 6A-6F, GSEA of genes involved in Oxphos and Mitochondrial protein import in human Alzheimer visual cortex expression datasets (GN327) (FIG. 6A) and whole brain (GN314) (FIG. 6D). Heatmaps of genes involved in Oxphos and mitochondrial protein import obtained from the GSEA analysis of the AD visual cortex (FIG. 6B) and whole brain (FIG. 6E) expression datasets. Low expression is shown in blue, while high expression is in red. Correlation plots of mitochondrial stress genes, UPR^{er} and HSR levels in human visual cortex (FIG. 6C) and whole brain (FIG. 6F) from AD patients subjects. (FIG. 6G) GSEA analysis of muscle transcript expression datasets from biopsies of healthy subjects and IBM patients (GSE3112 and GSE39454) showing a suggestive negative enrichment for mitochondrial function. Mito., mitochondrial.

FIGS. 7A-7G are graphs showing MSR analysis and mitochondrial function in 3×TgAD mice. (FIG. 7A) Transcript analysis of human APP expression in the cortex tissues of WT and 3×TgAD mice (WT n=4; AD n=4), confirming the identify of the AD model. (FIG. 7B) Transcript analysis of the MSR in the cortex tissues of WT (n=4) and 3×TgAD mice (n=4) at 6 months of age. (FIG. 7C) Immunoblot analysis (WT, n=5; 3×TgAD, n=6, WB of 4 representative mice) and quantification of selected mitochondrial proteostasis and autophagy effectors in cortex tissues of 6 months WT and 3×TgAD mice. (FIGS. 7D-7F) Transcript analysis of the MSR in the cortex tissues of WT (FIG. 7D, 6 mo, n=4; 9 mo, n=5) and 3×TgAD mice (FIG. 7E, 6 mo, n=4; 9 mo, n=5) at 6 and 9 months of age. The corresponding heatmaps (FIG. 7F) represent the relative variation in gene expression when comparing 6 and 9 months of age. The decrease in expression is more pronounced in the 3×TgAD mice. (FIG. 7G) CS activity assay performed on cortex tissue lysates from WT and 3×TgAD mice (WT, n=8; 3×TgAD, n=7). Values are mean±s.e.m. *P<0.05; **P≤0.01; ***P≤0.001; ***P≤0.001; n.s., non-significant.

FIGS. 8A-80 are graphs showing characterization of Aβ proteotoxicity and stress response pathways in the amyloid aggregation model GMC101. (FIG. 8A) Immunoblot of amyloid aggregation in CL2122 and GMC101 worms (n=3) maintained at 20° C., or after temperature shift to 25° C., at day 1 of adulthood. (FIG. 8B) Transcript analysis of the MSR in CL2122 and GMC101 maintained at 20° C. (n=3). (FIG. 8C) Respiration at basal level and after 45 min incubation with FCCP (10 μM) at day 1 and 3 of adulthood in CL2122 and GMC101 (n=10). Data are representative of at least two independent experiments. (FIG. 8D) CS activity assay performed on lysates from CL2122 and GMC101 on day 1 of adulthood (n=5 per group). (FIG. 8E) Mobility of worms from day 1 to 4 of adulthood in CL2122 and GMC101 (CL2122, n=24 to 48; GMC101, n=33 to 59). (FIG. 8F) Confocal images of CL2122 and GMC101 muscle cell integrity, nuclear morphology and mitochondrial networks, obtained respectively by phalloidin, DAPI and MitoTracker Orange CMTMros stainings at day 1 of adulthood. Scale bar, 10 μm. (FIG. 8G) Transcript analysis of UPR^{er}, HSR and daf-16 target genes in CL2122 and GMC101 after temperature shift, at day 1 of adulthood (n=3). (FIG. 8H) Representative images of CL2122 and GMC101 fed with RNAis targeting atfs-1, xbp-1 and hsf-1 after 4 days of development at 20° C. (n=2), and fraction of worms fed with these RNAis at the larval, L4 and adult stage after 4 days (n=2). (FIG. 8I) Validation of the atfs-1 RNAi in CL2122 and GMC101 by mRNA analysis at day 1 of adulthood (n=3). (FIG. 8J) Transcript analysis of the MSR in CL2122 fed with atfs-1 RNAi at day 1 of adulthood (n=3). (FIG. 8K) Mobility of GMC101 fed with 50% dilution of atfs-1 RNAi from day 1 to 4 of adulthood (ev, n=24 to 48; atfs-1^{1/2}, n=32 to 47). (FIG. 8L) Immunoblot of amyloid aggregation in CL2122 and GMC101 fed with atfs-1 RNAi and after temperature shift at day 1 of adulthood (n=3, WB representative of 2 biological replicates). (FIG. 8M) Mobility of worms at day 1 of adulthood in CL2122, GMC101, and N2 fed with atfs-1 or hsf-1 RNAi (CL2122, n=22 to 28; GMC101, n=18 to 27; N2, n=27 to 38). (FIG. 8N) Validation of the newly generated atfs-1#2 RNAi in CL2122 and GMC101 by mRNA analysis at day 1 of adulthood (n=3). (FIG. 8O) Mobility of worms from day 1 to day 4 of adulthood in CL2122 and GMC101 fed with the atfs-1#2 RNAi (CL2122, ev, n=36 to 47; atfs-1#2, n=38 to 42; GMC101, ev, n=30 to 55; atfs-1#2, n=39 to 46). ev, scrambled RNAi; A.U., arbitrary units. Values are mean±s.e.m. *P<0.05; **P≤0.01; ***P≤0.001; ****P≤0.0001; n.s., non-significant.

FIGS. 9A-9L are graphs showing reliance on ubl-5 and on increased mitochondrial stress response for survival and fitness of GMC101 worms. (FIG. 9A) Fraction of day 1 adult worms in CL2122 and GMC101 worms fed with ubl-5 RNAi after 4 days of development (n=3). (FIG. 9B) Mobility of worms from day 1 to day 4 of adulthood in CL2122 and GMC101 fed with the ubl-5 RNAi (CL2122, ev, n=33 to 39; ubl-5, n=36 to 43; GMC101, ev, n=40; ubl-5, n=35 to 41). (FIG. 9C) Percentage of paralyzed and dead CL2122 and GMC101 worms upon ubl-5 RNAi feeding at day 8 of adulthood (n=3). In FIGS. 9D-9E, Transcript analysis of UPR^{er}, HSR and daf-16 target genes in GMC101 (FIG. 9D) and CL2122 (FIG. 9E) fed with atfs-1 RNAi at day 1 of adulthood (n=3). (FIG. 9F) Validation of the atfs-1 overexpressing strains AUW9, AUW10 and AUW11 by assessment of the mRNA levels of atfs-1 and its targets hsp-6 and hsp-60 in these strains, and in GMC101 and CL2122 (n=3). (FIG. 9G) Mobility of worms from day 1 to 4 of adulthood in control and atfs-1 overexpressing CL2122 (AUW11), and control and atfs-1 overexpressing GMC101 (AUW9, AUW10) lines (CL2122, n=34 to 40; GMC101, n=41 to 57; AUW9, n=36 to 40; AUW10, n=36 to 38; AUW11, n=34 to 42). (FIG. 9H) Percentage of paralyzed and dead GMC101, AUW9 and AUW10 at day 6 of adulthood (n=3). (FIG. 9I) Mobility of worms from day 1 to 5 of adulthood in GMC101, the clk-1 mutant strain CB4876, and the AUW12 strain, derived from the cross between GMC101 and CB4876 (GMC101, n=34 to 35; CB4876 n=29 to 42; AUW12, n=34 to 38). (FIG. 9J) Percentage of paralyzed and dead GMC101, CB4876 and AUW12 worms at day 8 of adulthood (n=3). (FIG. 9K) Mobility of worms from day 1 to 4 of adulthood in, GMC101, the nuo-6 mutant strain MQ1333, and the AUW13 strain, derived from the cross between GMC101 and MQ1333 (GMC101, n=43 to 46; MQ1333 n=32 to 50; AUW13, n=34 to 47). (FIG. 9L) Percentage of paralyzed and dead GMC101, MQ1333 and AUW13 worms at day 8 of adulthood (n=3). ev, scrambled RNAi; A.U., arbitrary units. Values are mean±s.e.m. *P<0.05; **P≤0.01; ***P≤0.001; ****P≤0.0001; n.s., non-significant.

FIGS. 10A-10O are graphs showing effects of the inhibition of mitochondrial translation and mitophagy in control and GMC101 worms, and of compound treatments in mammalian cells. (FIG. 10A) Representative images of GMC101 worms fed with mrps-5 RNAi or treated with dox (15 μg/mL) from eggs to day 1 of adulthood (n=2). (FIG. 10B) Transcript analysis of the MSR of CL2122 (n=4) worms treated with dox (15 μg/mL) at day 1 of adulthood. In FIGS. 10C-10D, Transcript analysis of UPR^{er}, HSR and daf-16 target genes in GMC101 fed with mrps-5 RNAi (FIG. 10C), or treated with dox (15 μg/mL) (FIG. 10D) at day 1 of adulthood (n=3). (FIG. 10E) Immunoblot of amyloid aggregation in CL2122 and GMC101 fed with mrps-5 RNAi or treated with dox (15 μg/mL) at day 1 of adulthood (n=3). (FIG. 10F) Respiration at basal level at day 3 and 6 of adulthood in GMC101 worms fed with mrps-5 RNAi (n=8-10). (FIG. 10G) Additional confocal images of the intracellular amyloid deposits in the SH-SY5Y_{(APPSwe)} neuroblastoma cell line stained with the anti-β-Amyloid 1-42 (Millipore AB5078P) antibody, after treatment with dox (10 μg/mL) and, where indicated, ISRIB (0.5 μM) for 24 h. Scale bar, 10 μm. Data are representative of at least two independent experiments. (FIG. 10H) Western-blot analysis showing that NR (1 mM) increases oxidative phosphorylation (OXPHOS) proteins levels, and that dox (10 μg/mL) increases the ratio of nDNA-(ATP5A, UQCRC1) over mtDNA-encoded (MTCO1) OXPHOS proteins in SH-SY5Y_{(APPSwe)} cells (n=2). (FIG. 10I) Transcript levels of representative MSR genes in the APP_{Swe}-expressing cell line after 24 h of dox (10 μg/mL; n=4). (FIG. 10J) Transcript analysis of SH-SY5Y_{(APPSwe)} cells treated as in FIG. 10G, of the ATF4 target genes CHOP and CHAC1 (n=4). (FIG. 10K) Mobility of GMC101 fed with dct-1 RNAi from day 1 to 4 of adulthood (ev, n=34 to 54; dct-1, n=42 to 44). (FIG. 10L) Mobility of worms from day 1 to 4 of adulthood in GMC101 fed with dct-1, mrsp-5, or both RNAis (ev, n=34 to 54; dct-1, n=42 to 44; mrps-5, n=34 to 35; mrps-5, dct-1, n=41 to 45). (FIG. 10M) Mobility of worms from day 1 to 4 of adulthood in GMC101 treated with dox or fed with dct-1 RNAi (ev, n=34 to 54; dct-1, n=42 to 44; dox, n=43 to 52; dox, dct-1, n=34 to 54). In FIGS. 10N-10O, Mobility of CL2122 fed with dct-1 RNAi from (FIG. 10N) day 1 to 4 of adulthood (ev, n=32 to 44; dct-1, n=38 to 40), or (FIG. 10O) at day 8 of adulthood (n=38). ev, scrambled RNAi; dox., doxycycline; A.U., arbitrary units. Values are mean±s.e.m. *P<0.05; **P≤0.01; ***P≤0.001; ****P≤0.0001 n.s., non-significant.

FIGS. 11A-11L are graphs showing effect of NAD⁺-boosting compounds and sirtuin depletion in control and GMC101 worms, and NR treatment in mammalian cells. In FIGS. 11A-11B, transcript analysis of the MSR of CL2122 treated with NR (1 mM) (FIG. 11A, n=3 or AZD (0.3 μM) (FIG. 11B, n=3 at day 1 of adulthood. In FIGS. 11C-11D, Mobility of CL2122 treated with NR (1 mM) and AZD (0.3 μM) from (FIG. 11C) day 1 to 4 of adulthood (vehicle, n=n=32 to 44; NR, n=32 to 48; AZD, n=39 to 43), or (FIG. 11D) at day 8 of adulthood (vehicle, n=38; NR, n=36; AZD, n=33). (FIG. 11E) Representative images of CL2122 and GMC101 fed with RNAis for atfs-1, sir-2.1, and daf-16 after 4 days of development (n=2). (FIG. 11F) Mobility of worms from day 1 to 4 of adulthood in GMC101 treated with NR (1 mM) and fed with sir-2.1 (ev, n=31 to 52; sir-2.1, n=37; NR, n=32 to 40; sir-2.1; NR, n=38 to 51). (FIG. 11G) Mobility of worms from day 1 to 4 of adulthood in GMC101 treated with NR (1 mM) and fed with daf-16 (ev, n=31 to 52; daf-16, n=27 to 43; NR, n=32 to 40; daf-16; NR, n=38 to 48). (FIG. 11H) Percentage of paralyzed and dead GMC101 treated with NR or fed with sir-2.1, daf-16 or atfs-1 RNAis at day 8 of adulthood (n=3). In FIGS. 11I-11J, Transcript analysis of UPR^{er}, HSR and daf-16 target genes in GMC101 treated with NR (1 mM) (FIG. 11D), or AZD (0.3 μM) (FIG. 11E) at day 1 of adulthood (n=3). (FIG. 11K) Additional confocal images of the intracellular amyloid deposits in the SH-SY5Y_{(APPSwe)} cell line stained with the anti-β-Amyloid 1-42 antibody, after treatment with NR (3 mM) for 24 h. (FIG. 11L) Transcript levels of representative MSR genes in the APP_{Swe}-expressing cell line after treatment with NR (1 mM) for 24 h (n=4). Data are representative of at least two independent experiments. NR, nicotinamide riboside; CD, chow diet; ISRIB, integrated stress response inhibitor; AZD, Olaparib; ev, scrambled RNAi; A.U., arbitrary units. Values are mean±s.e.m. *P<0.05; **P≤0.01; ***P≤0.001; ****P≤0.0001; n.s., non-significant.

FIGS. 12A-12G are graphs showing targeted mass spectrometry-based identification of endogenous Aβ peptide in non-transgenic mice, NR effects in aged mice, and proposed model. (FIG. 12A) Schematic of the APP protein, with the C-terminal region containing the membrane-spanning domain and the Aβ peptides identified via LC-MS/MS in mouse brain tissues. The Aβ1-42 peptide is shown below as part of APP. Aβ1-40 or the longer form of Aβ1-42 are produced by the sequential cleavage of APP by β- and γ-secretases, of which the activity of the latter enzyme results in cleavage of the two different C-terminal isoforms of Aβ1-40 and the more amyloidogenic form Aβ1-42, respectively. Different Aβ cleavage products may also be observed as a result α-Secretase activity, which cleaves APP in the middle of the Aβ peptide domain, generating Aβ fragments lacking the N-terminal domain of residues Aβ1-16. For qualitative identification of Aβ fragments in mouse brains, 4G8-immunocomplexes from old C57BL/6J mice (n=1 per group) brain extracts were digested using Lys-N protease and analyzed by LC-MS/MS. The cleavage specificity of Lys-N protease generates fragments of residues Aβ1-15 and Aβ16-27 as well as the C-terminal fragment of Aβ28-42. The Aβ peptide fragments identified using the combination of IP (4G8) and LC-MS/MS are highlighted in red. The lack of MS detection of other, non-Aβ, proteotypic peptides associated with the APP sequence, together with the presence of Aβ peptide starting at residue Asp1 strongly suggests that the here reported identification of endogenous murine Aβ peptide is the result of a specific pull-down (IP-4G8) of Aβ peptide from muscle and brain tissues. (FIG. 12B) Tandem (LC-MS/MS) spectrum of the proteolytic (Lys-N) fragment Aβ16-27 showing the unbiased identification of several b-ions (blue) and y-ions (red). The bottom graph indicates the measured fragment ion mass error relative to the calculated theoretical mass. Similar sequence coverage was achieved for Aβ1-15 (data not shown). (FIG. 12C) Multiple reaction monitoring (MRM) summary of peptide transitions (as a sum of transition peak area) detected for fragment Aβ16-27 in young (left), old (centre) and AD (right) mouse brain (top row), as well as forelimbs (bottom row) (n=1 per group). The MRM approach resulted in the qualitative identification of endogenous Aβ from brain (top) and muscle (bottom) tissue from aged and AD animals, whereas detection of Aβ in forelimbs from young animals was below the analytical limit of detection (for the sake of clarity and comparison only two abundant peptide transitions are labelled: b9 and b8). (FIG. 12D) Overview of MRM peptide transition spectrum of all transitions monitored for mouse endogenous Aβ16-27 (top; aged mouse muscle tissue) and the synthetic heavy (K) labelled surrogate peptide (bottom). The identical LC retention time and MS ionization properties, as well as the transition fragment signature (b-ions) allowed for an unbiased identification of endogenous Aβ peptide in muscle tissue. Heavy labelled transitions bearing the heavy lysine (K) residue within the sequence are labelled in red (b-ions) and show a mass difference of 8 Da (heavy K), whereas light (endogenous) peptide transitions are labelled in black. In FIGS. 12E-12F, transcript analysis of the MSR in quadriceps tissues from young (n=4) (FIG. 12E) and old (FIG. 12F) C57BL/6J mice with or without NR treatment (n=5). Values are mean±s.e.m. *P<0.05; **P≤0.01; ***P≤0.001. CD, chow diet. (FIG. 12G) Proposed model for the GMC101 worm strain illustrating the critical role of mitochondrial homeostasis and the benefits of enhancing mitochondrial proteostasis in Aβ proteopathies. (1) Accumulation of amyloid aggregates triggers mitochondrial dysfunction, which induces the MSR (2) Depletion of atfs-1 results in loss of mitochondrial homeostasis, which leads to faster and more pronounced amyloid aggregation and decreased healthspan. (3) Enhancing mitochondrial proteostasis in GMC101 worms with dox, mrps-5 RNAi, and NAD⁺ boosters (e.g. NR and Olaparib), leads to a recovery of organismal fitness, delaying the development of the signs of Aβ diseases.

FIGS. 13A-13D are graphs showing that inhibition of sphingosine biosynthesis pathway protects from proteotoxic Aβ disease in worms. (FIG. 13A) Treatment of GMC101 worms with increasing doses of Myriocin (5 uM and 10 uM), a potent inhibitor of serine palmitoyltransferase, results in a dose-dependent reduction in accumulation of amyloid-β aggregates (c13) in GMC101 worms. (FIG. 13B) Myriocin treatment at the concentration of 10 uM also results in an increase in spontaneous movement in GMC101 worms, and (FIG. 13C) improves the paralysis score and survival at day 4 of adulthood. (FIG. 13D) Treatment of GMC101 worms with increasing doses of myriocin (5 uM and 10 uM) leads to the induction of genes and transcription factors involved in oxidative stress and mitochondrial stress response, such as sod-2, sod-4, atfs-1, skn-1. Additionally, myriocin treatment results in upregulation of UPR^{mt} and mitophagy genes, such as hsp-6, hsp-60, dct-1 and pdr-1.

FIG. 14A is a graph showing that inhibition of ceramide synthesis resolves Aβ proteotoxic stress in human cells. Treatment of human neuroblastoma cell line expressing the APP (Amyloid beta Precursor Protein) Swedish K670N/M671L double mutation with Myriocin (serine palmitoyltransferase inhibitor) and Fumonisin B1 (ceramide synthase inhibitor), both at 10 uM, markedly reduced intracellular Aβ deposits, as shown by immunodetection with an Aβ1-42-specific antibody. FIG. 14B shows the experimental pipeline for the treatment of aged mice (18 months of age or older) with Myriocin, at 0.4 mg/kg via intra peritoneal injection for 8 weeks. (FIG. 14C) Myriocin, administered as described in FIG. 14B, improves grip strength, exercise capacity (distance run), balance (latency square in beamwalk), and movement coordination (rotarod latency) in old mice. (FIG. 14D) Myriocin treatment reduces amyloid deposits, as shown by immunodetection with Thioflavin S (ThS), in the muscle tissues of old mice.

FIGS. 15A-15B are graphs showing that enhancing mitophagy with Urolithin A (UA) increases proteostasis and fitness in the GMC101 worms. (FIG. 15A) Treatment of GMC101 with increasing doses of UA (20 uM and 50 uM), a natural compound that increases clearance of damaged mitochondria via mitophagy in many organisms, results in a dose-dependent increase in spontaneous movement in affected worms. (FIG. 15B) UA treatment also results in a dose-dependent reduction in accumulation of amyloid-β aggregates (c13) in GMC101 worms and therefore improved organismal proteostasis, in agreement with the increased fitness observed in panel A.

### DETAILED DESCRIPTION OF THE INVENTION

### Definitions

“Treat,” “treating,” or “treatment” refers to decreasing the symptoms, markers, and/or any negative effects of a disease or condition in any appreciable degree in a subject who currently has the disease or condition. In some embodiments, treatment may be administered to a subject who exhibits only early signs of a disease or condition for the purpose of decreasing the risk of developing the disease or condition. In some embodiments, “treat,” “treating,” or “treatment” refers to amelioration of one or more symptoms of a disease or condition. For example, amelioration of one or more symptoms of a disease or condition includes a decrease in the severity, frequency, and/or length of one or more symptoms of a disease or condition.

“Prevent,” “prevention,” or “preventing” refers to any method to partially or completely prevent or delay the onset of one or more symptoms or features of a disease or condition. Prevention may be administered to a subject who does not exhibit any sign of a disease or condition.

“Subject” means a human or animal (in the case of an animal, more typically a mammal). In some embodiments, the subject is a human.

“Therapeutically effective amount” refers to that amount of a drug or pharmaceutical agent that will elicit the biological or medical response of a tissue, system, animal, or human that is being sought by a researcher or clinician.

“Pharmaceutical” or “pharmaceutically acceptable” when used herein as an adjective, means substantially non-toxic and substantially non-deleterious to the recipient.

As used herein, the term “inhibit” means to reduce an activity by at least 10%, and preferably more, e.g., 20%, 30%, 40%, 50%, 60%, 70%, 80%, 90% or more, up to and including 100% relative to that activity that is not subject to such inhibition.

As used herein, the term “enhance” means to increase an activity by at least 10%, and preferably more, e.g., 20%, 50%, 75% or even 100% or more (e.g., 2×, 5×, 10×, etc.) relative to that activity that is not subject to such enhancement.

As used herein, the term “small molecule” refers to organic molecules, whether naturally-occurring or artificially created (e.g., via chemical synthesis) that have relatively low molecular weight and that are not proteins, polypeptides, or nucleic acids. Typically, small molecules have a molecular weight of less than about 1500 g/mol.

The term “about” refers to a range of values which can be 15%, 10%, 8%, 5%, 3%, 2%, 1%, or 0.5% more or less than the specified value. For example, “about 10%” can be from 8.5% to 11.5%. In one embodiment, the term “about” refers to a range of values which are 5% more or less than the specified value. In another embodiment, the term “about” refers to a range of values which are 2% more or less than the specified value. In another embodiment, the term “about” refers to a range of values which are 1% more or less than the specified value.

The articles “a” and “an” are used in this disclosure to refer to one or more than one (i.e., to at least one) of the grammatical object of the article. By way of example, “an element” means one element or more than one element.

The term “and/or” is used in this disclosure to mean either “and” or “or” unless indicated otherwise.

The present invention is based, inter alia, on the discovery that amyloid-3 peptide proteopathies perturb mitochondria and repairing mitochondrial proteostasis reduces protein aggregation in animal models of amyloid-3 diseases. Accordingly, one aspect of the invention relates to a method of treating an amyloid-3 peptide disease in a subject in need thereof, the method comprising administering to the subject a therapeutically effective amount of a compound that enhances mitochondrial proteostasis. Enhancement of mitochondrial proteostasis can be measured using two complementary strategies: (1) by assessing the expression levels of biomarkers of mitochondrial stress response in cells and biopsies material, such as measuring transcript and/or protein levels of mitochondrial stress response, e.g. LONP1, CLPP, HSPA9 and HSP60; and/or (2) by determining the levels of biomarkers of mitochondrial stress in the plasma, the so-called “mitokines”, such as FGF21 and GDF 15.

In some embodiments, the compound enhances mitochondrial proteostasis by at least 10%, at least 20%, at least 30%, at least 40%, at least 50%, at least 60%, at least 70%, at least 80%, at least 90% as compared to a control when the compound is not administered or a placebo is administered.

The amyloid-3 peptide disease can be a muscle disease, a metabolic disease, Alzheimer's disease, Dementia, Parkinson's disease, Huntington's disease, or amyotropic lateral sclerosis. In some embodiments, the muscle disease is inclusion body myositis, age-related sarcopenia, frailty of the elderly, or muscular dystrophy (e.g., Duchenne's or Becker's muscular dystrophy). In some embodiments, the metabolic disease is type 2 diabetes, an amyloid kidney disease, or an amyloid heart disease.

In some embodiments, the amyloid-3 peptide disease is Alzheimer's disease.

Symptoms of Alzheimer's disease include memory loss, confusion, irritability, aggression, mood swings and trouble with language. This disease is characterized by the loss of neurons and synapses in the cerebral cortex and certain subcortical regions. The loss results in gross atrophy of the affected regions, including degeneration in the temporal lobe, and parts of the frontal cortex and cingulate gyrus. Amyloid plaques and neurofibrillary tangles are visible by microscopy in brains of those afflicted with this disease. Alzheimer's disease is usually diagnosed based on the person's medical history, history from relatives, and behavioural observations. Advanced medical imaging with computed tomography (CT) or magnetic resonance imaging (MRI), and with single-photon emission computed tomography (SPECT) or positron emission tomography (PET) can be used to help exclude other cerebral pathology or subtypes of dementia.

In some embodiments, the amyloid-β peptide disease is inclusion body myositis. Symptoms of inclusion body myositis include weakening of muscles leading to finger flexion, loss of balance/control leading to tripping and falling occasionally, climbing stairs difficulties, climbing stairs difficulties, difficulty bending down, loss of mobility, off balance posture, Low tolerance for severe injuries, and leg muscles being unstable. Certain blood tests and/or a muscle biopsy may be performed for the diagnosis of inclusion body myositis.

The compound that enhances mitochondrial proteostasis can be a small molecule, a peptide, a polypeptide, or a polynucleotide. The compound can be either synthetic or natural. For example, the compound can be a pharmaceutical compound. The compound can also be a natural product that serve as a nutraceutical. Urolithin A, nicotinamide riboside, nicotinamide mononucleotide, nicotinic acid, nicotinamide, and quercetin are non-limiting examples of natural products that can be used for the methods of the present disclosure.

In some embodiments, the compound that enhances mitochondrial proteostasis can be a compound that induces mitochondrial unfolded protein response (UPR^{mt}). Compounds that induce UPR^{mt} can include, but are not limited to, tetracycline, chlortetracycline, oxytetracycline, demeclocycline, lymecycline, meclocycline, methacycline, minocycline, rolitetracycline, doxycycline, tigecycline, actinonin, chloramphenicol, and a compound that blocks mitochondrial import. In some embodiments, a compound that induces UPR^{mt} can be doxycycline.

In some embodiments, the compound that enhances mitochondrial proteostasis can be a compound that induces mitochondrial biogenesis. Compounds that induce mitochondrial biogenesis can include, but are not limited to, an NAD⁺ boosting compound, a PARP inhibitor, a CD38 or CD157/BST1 inhibitor, an activator of nicotinamide phosphoribosyltransferases (NAMPT), an inhibitor of nicotinamide N methyltransferases (NNMT), and an inhibitor of α-amino-3-carboxymuconate-ε-semialdehyde decarboxylase (ACMSD).

In some embodiments, an NAD⁺ boosting compound can be an NAD⁺ precursor, such as nicotinamide riboside, nicotinamide mononucleotide, nicotinic acid, and nicotinamide.

Exemplary PARP inhibitors include, but are not limited to, olaparib, rucaparib, niraparib, iniparib, talazoparib, veliparib, CEP 9722, Eisai's E7016, BGB-290, and 3-aminobenzamide.

Exemplary CD38 or CD157/BST1 inhibitors include, but are not limited to, GSK 897-78c, apigenin, and quercetin. Exemplary activators of NAMPT include, but are not limited to, PC73.

Exemplary inhibitors of ACMSD include, but are not limited to, a phthalate ester and pyrazinamide. Common phthalate esters include dimethyl phthalate, diethyl phthalate, diallyl phthalate, di-n-propyl phthalate, di-n-butyl phthalate, diisobutyl phthalate, butyl cyclohexyl phthalate, di-n-pentyl phthalate, dicyclohexyl phthalate, butyl benzyl phthalate, di-n-hexyl phthalate, diisohexyl phthalate, diisoheptyl phthalate, butyl decyl phthalate, di(2-ethylhexyl) phthalate, di(n-octyl) phthalate, diisooctyl phthalate, n-octyl n-decyl phthalate, diisononyl phthalate, di(2-propylheptyl) phthalate, diisodecyl phthalate, diundecyl phthalate, diisoundecyl phthalate, ditridecyl phthalate, and diisotridecyl phthalate.

In some embodiments, the compound that enhances mitochondrial proteostasis is a compound that induces mitophagy. Compounds that induce mitophagy can include, but are not limited to, Urolithin A, Urolithin B, 5-aminoimidazole-4-carboxamide-ribonucleoside, salicylate, 6,7-dihydro-4-hydroxy-3-(2′-hydroxy[1,1′-biphenyl]-4-yl)-6-oxo-thieno[2,3-b]pyridine-5-carbonitrile (i.e., A-769662), 1-(2,6-Dichlorophenyl)-6-[[4-(2-hydroxyethoxy)phenyl]methyl]-3-propan-2-yl-2H-pyrazolo[3,4-d]pyrimidin-4-one (i.e., AMPK Activator 991), and metformin.

In some embodiments, the compound that enhances mitochondrial proteostasis can be a compound that modulates lipid metabolism. Compounds that modulate lipid metabolism can include, but are not limited to, perhexiline, a fibrate, and a statin. Exemplary fibrates include, but are not limited to, aluminium clofibrate, bezafibrate, ciprofibrate, choline fenofibrate, clinofibrate, clofibrate, clofibride, fenofibrate, gemfibrozil, ronifibrate, and simfibrate. In some embodiments, the fibrate is fenofibrate, clofibrate, or bezafibrate. Exemplary statins include, but are not limited to, atorvastatin, cerivastatin, fluvastatin, lovastatin, mevastatin, pitavastatin, pravastatin, rosuvastatin, and simvastatin. In some embodiments, the statin is lovastatin, simvastatin, atorvastatin, or fluvastatin.

In some embodiments, a compound that modulates lipid metabolism is a compound that inhibits sphingosin and ceramide synthesis. Exemplary compounds that inhibit sphingosin and ceramide synthesis include, but are not limited to, myriocin, fumonisin B1, alverine, amiodarone, amitriptyline, aprindine, AY-9944, biperiden, camylofin, carvedilol, cepharanthine, chlorpromazine, chlorprothixene, cinnarizine, clemastine, clofazimine, clomiphene, clomipramine, cloperastine, conessine, cyclobenzaprine, cyproheptadine, desipramine, desloratadine, dicycloverine, dicyclomine, dilazep, dimebon, drofenine, emetine, fendiline, flunarizine, fluoxetine, flupentixol, fluphenazine, fluvoxamine, hydroxyzine, imipramine, lofepramine, loperamid, loratadin, maprotiline, mebeverine, mebhydrolin, mepacrine, mibefradil, norfluoxetine, nortriptyline, paroxetine, penfluridol, perhexiline, perphenazine, pimethixene, pimozide, profenamine, promazine, promethazine, protriptyline, sertindole, sertraline, solasodine, suloctidil, tamoxifen, terfenadine, thioridazine, tomatidine, trifluoperazin, triflupromazine, trimipramine, zolantidine, 3-O-ethyl 5-O-methyl 2-(2-aminoethoxymethyl)-4-(2-chlorophenyl)-6-methyl-1,4-dihydropyridine-3,5-dicarboxylate (i.e., amlodipine), 1-[(4-fluorophenyl)methyl]-N-[1-[2-(4-methoxyphenyl)ethyl]-4-piperidyl]benzoimidazol-2-amine (i.e., astemizole), (1R,5S)-3-benzhydryloxy-8-methyl-8-azabicyclo[3.2.1]octane (i.e., benztropine), N-benzyl-N-[3-(2-methylpropoxy)-2-pyrrolidin-1-ylpropyl]aniline (i.e., bepridil), and (3E)-3-(6H-benzo[c][1]benzoxepin-11-ylidene)-N,N-dimethylpropan-1-amine (i.e., doxepine). In some embodiments, a compound that inhibits sphingosin and ceramide synthesis is myriocin, fumonisin B1, amlodipine, astemizole, benztropine, bepridil, or doxepine.

A compound that enhances mitochondrial proteostasis can be administered via any administration routes, including oral administration in forms such as tablets, capsules (each of which includes sustained release or timed release formulations), pills, powders, granules, elixirs, tinctures, suspensions, syrups emulsions, intravenous administration (bolus or in-fusion), intraperitoneal administration, topical administration (e.g., ocular eye-drop), subcutaneous administration, intramuscular administration, transdermal (e.g., patch) administration, and intravitreal administration.

Administration of the compound that enhances mitochondrial proteostasis typically is carried out over a defined time period, e.g., days, weeks, or months. The dosage regimen utilizing the compounds of the invention is selected in accordance with a variety of factors including type, species, age, weight, sex and medical condition of the patient; the severity of the condition to be treated; and the particular compound or salt thereof employed. An ordinary skilled physician, veterinarian or clinician can readily determine and prescribe the effective amount of the drug required to prevent, counter or arrest the progress of the condition. Examples of dosing regimens that can be used in the methods of the invention include, but are not limited to, daily, three times weekly (intermittent), weekly, or every 14 days.

In one embodiment, the dose is from about 0.2 to 1000 mg/kg of body weight, depending on the specific compound being used. In some embodiments, the dosage is about 0.2 to 800 mg/kg of body weight, 0.2 to 500 mg/kg of body weight, 10 to 500 mg/kg of body weight, or 10 to 300 mg/kg of body weight. In some embodiments, the dosage is about 50 mg/kg, 60 mg/kg, 70 mg/kg, 80 mg/kg, 90 mg/kg, 100 mg/kg, 150 mg/kg, 200 mg/kg, 250 mg/kg, 300 mg/kg, 350 mg/kg, 400 mg/kg, 450 mg/kg, 500 mg/kg, 550 mg/kg, 600 mg/kg, 650 mg/kg, 700 mg/kg, 750 mg/kg, 800 mg/kg, 850 mg/kg, 900 mg/kg, 950 mg/kg, or 1000 mg/kg of body weight.

In some embodiments, doxycycline is administered at about 90 mg/kg/day in food for at least 4 weeks, e.g., 8 weeks, 9 weeks, 10 weeks, 12 weeks, or 14 weeks. In some embodiments, doxycycline is administered at about 200-500 mg/kg/day in water for at least 4 weeks, e.g., 8 weeks, 9 weeks, 10 weeks, 12 weeks, or 14 weeks.

In some embodiments, nicotinamide riboside is administered at about 400 mg/kg/day in food for at least 4 weeks, e.g., 8 weeks, 9 weeks, 10 weeks, 12 weeks, or 14 weeks.

In some embodiments, myriocin is administered at 0.4 mg/kg of body weight, for example, three times per week by injection.

In some embodiments, olaparib or velaparib is administered at 300 mg/kg/day in food for about 9-10 weeks.

In some embodiments, a therapeutically effective amount of the compound can reduce amyloid-β formation by at least 10%, at least 20%, at least 30%, at least 40%, at least 50%, at least 60%, at least 70%, at least 80%, or at least 90%.

In the methods of the invention, the compounds herein described in detail can form the active ingredient, and are typically administered in admixture with suitable pharmaceutical diluents, excipients or carriers (collectively referred to herein as “carrier”). Pharmaceutically acceptable carriers useful in the composition include, for example, propylene glycol, polypropylene glycol, polyethylene glycol (e.g. PEG 400), glycerol, ethanol, dimethyl isosorbide, glycofurol, propylene carbonate, dimethyl acetamide, water, or mixtures thereof; preferably, propylene glycol, polyethylene glycol, ethanol, water, or mixtures thereof. Examples of excipients include certain inert proteins such as albumins; hydrophilic polymers such as polyvinylpyrrolidone; amino acids such as aspartic acid (which may alternatively be referred to as aspartate), glutamic acid (which may alternatively be referred to as glutamate), lysine, arginine, glycine, and histidine; fatty acids and phospholipids such as alkyl sulfonates and caprylate; surfactants such as sodium dodecyl sulphate and polysorbate; nonionic surfactants such as such as TWEEN®, PLURONICS®, or polyethylene glycol (PEG); carbohydrates such as glucose, sucrose, mannose, maltose, trehalose, and dextrins, including cyclodextrins; polyols such as mannitol and sorbitol; chelating agents such as EDTA; and salt-forming counter-ions such as sodium.

The compounds disclosed herein can be administered in a combination therapy. For example, two or more compounds can be administered simultaneously or sequentially. In some embodiments, the two or more compounds can be administered at the optimal dose respectively. In some embodiments, one of the compounds can be administered at a suboptimal dose, for example, to minimize or avoid side effects. In some embodiments, the two or more compounds can be administered at a suboptimal dose respectively. In some embodiments, a natural product can be administered in combination with a synthetic compound, for example, to optimize the efficacy of both while minimizing the side effects of the synthetic compound.

The details of the invention are set forth in the accompanying description below. Although methods and materials similar or equivalent to those described herein can be used in the practice or testing of the present invention, illustrative methods and materials are now described. Other features, objects, and advantages of the invention will be apparent from the description and from the claims. In the specification and the appended claims, the singular forms also include the plural unless the context clearly dictates otherwise. Unless defined otherwise, all technical and scientific terms used herein have the same meaning as commonly understood by one of ordinary skill in the art to which this invention belongs. All patents and publications cited in this specification are incorporated herein by reference in their entireties.

### EXAMPLES

The disclosure is further illustrated by the following examples and synthesis examples, which are not to be construed as limiting this disclosure in scope or spirit to the specific procedures herein described. It is to be understood that the examples are provided to illustrate certain embodiments and that no limitation to the scope of the disclosure is intended thereby. It is to be further understood that resort may be had to various other embodiments, modifications, and equivalents thereof which may suggest themselves to those skilled in the art without departing from the spirit of the present disclosure and/or scope of the appended claims.

### Example 1

Alzheimer's disease (AD) and inclusion body myopathy (IBM) are common and devastating diseases characterized by the aggregation of amyloid-β peptide (Aβ), yet we know relatively little about their underlying molecular mechanisms or how to treat them. Here, we provide bioinformatic and experimental evidence of a conserved mitochondrial stress response signature present in Aβ proteotoxic diseases in human, mouse and C. elegans, and which involves the UPR^{mt} and mitophagy pathways. Using the worm model of Aβ proteotoxicity, GMC101, we recapitulated mitochondrial features and confirmed the induction of this mitochondrial stress response as key to maintain mitochondrial proteostasis and health. Importantly, boosting mitochondrial proteostasis by pharmacologically and genetically targeting mitochondrial translation and mitophagy increases fitness and lifespan of GMC101 worms and reduces amyloid aggregation in cells, worms, and in aged and AD transgenic mice. Our data support the relevance of enhancing mitochondrial proteostasis to delay Aβ proteotoxic diseases, such as AD and IBM.

Mitochondrial function and proteostasis are perturbed in Aβ diseases.

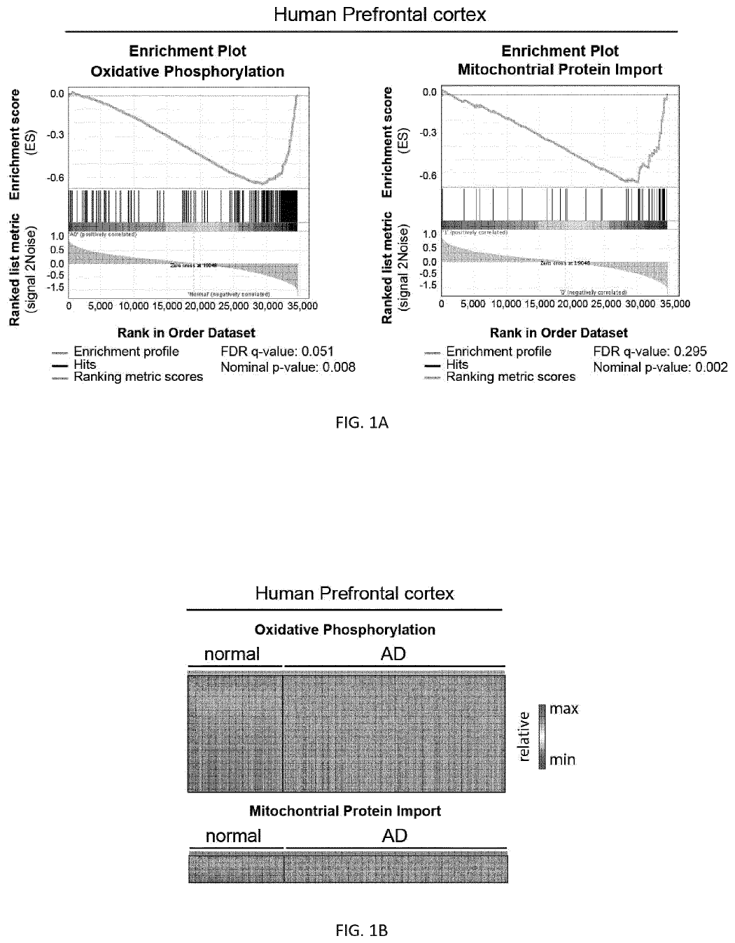

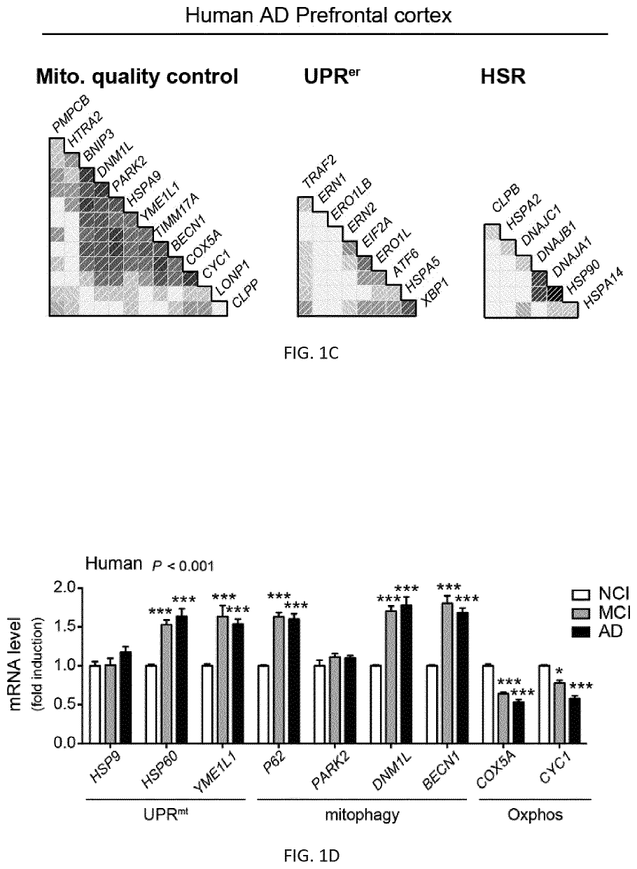

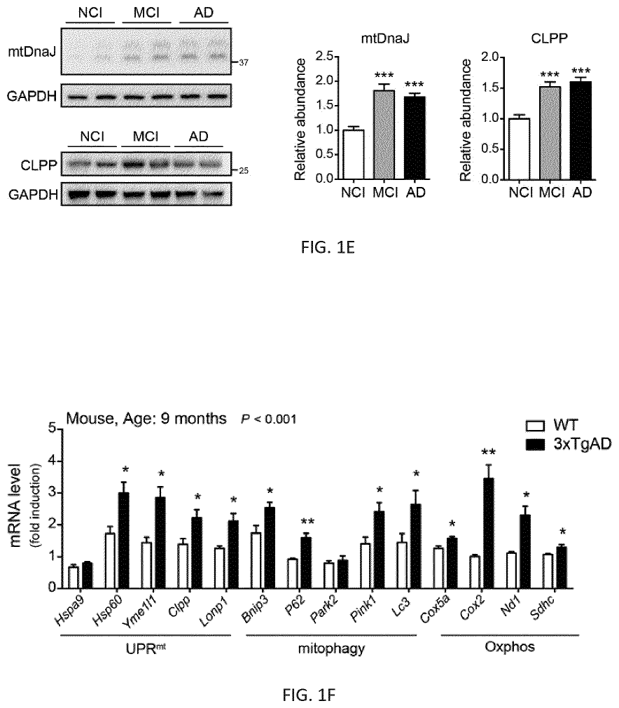

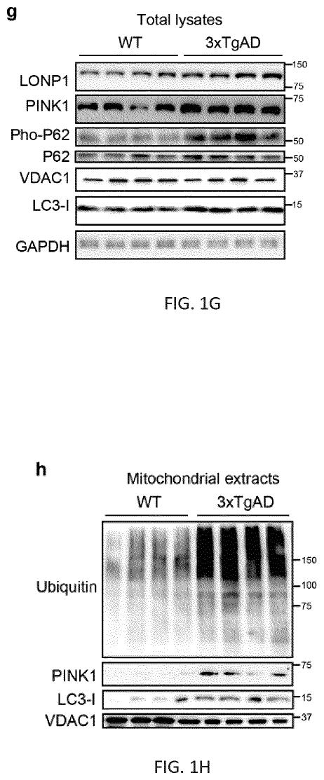

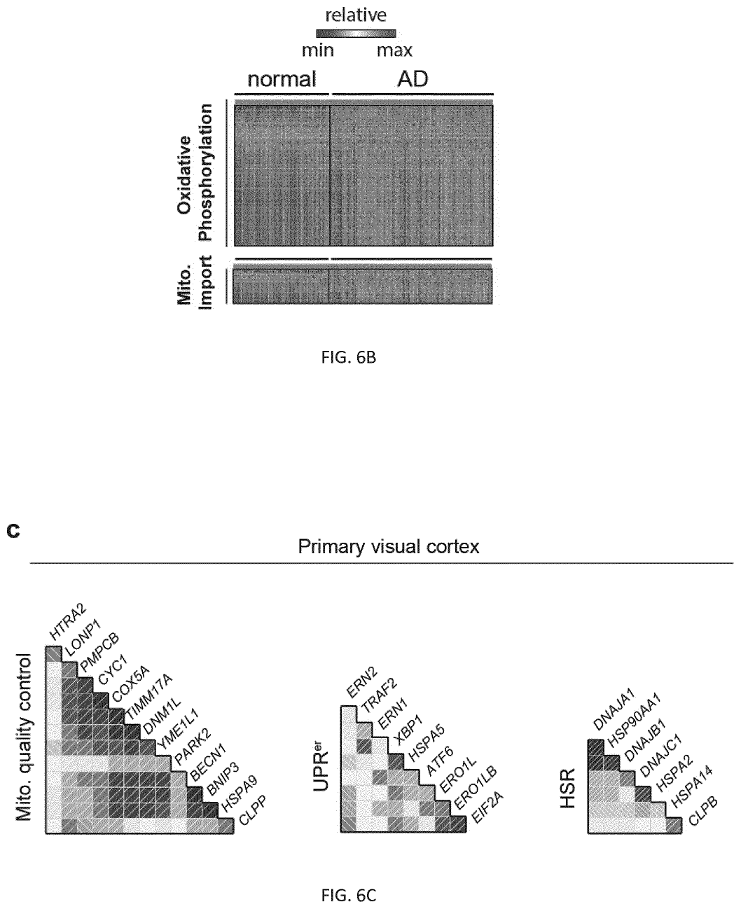

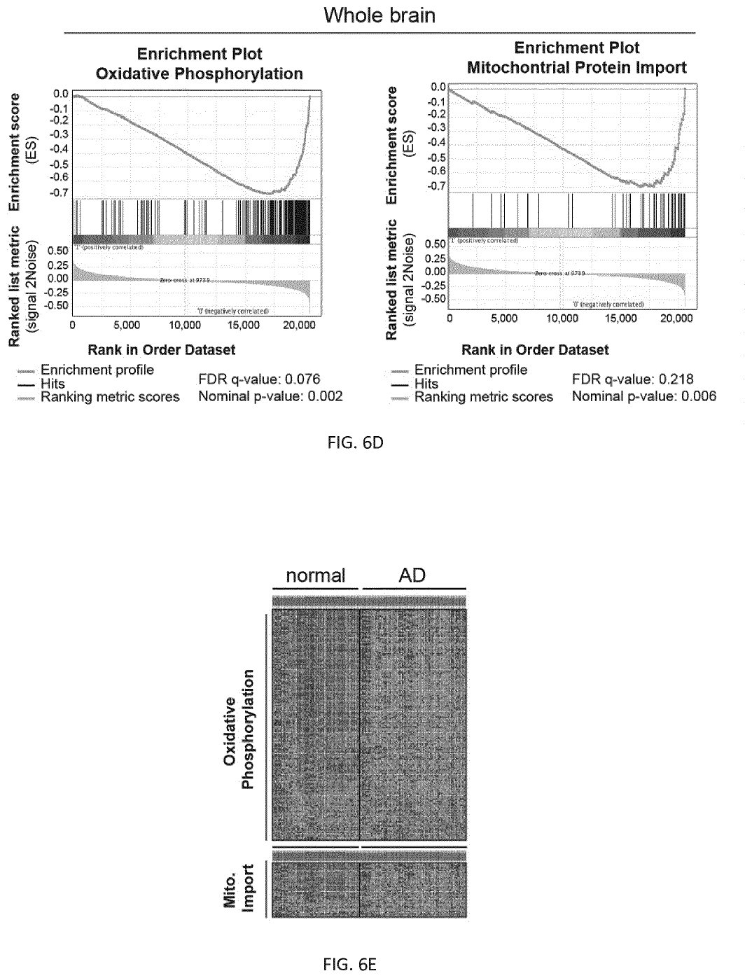

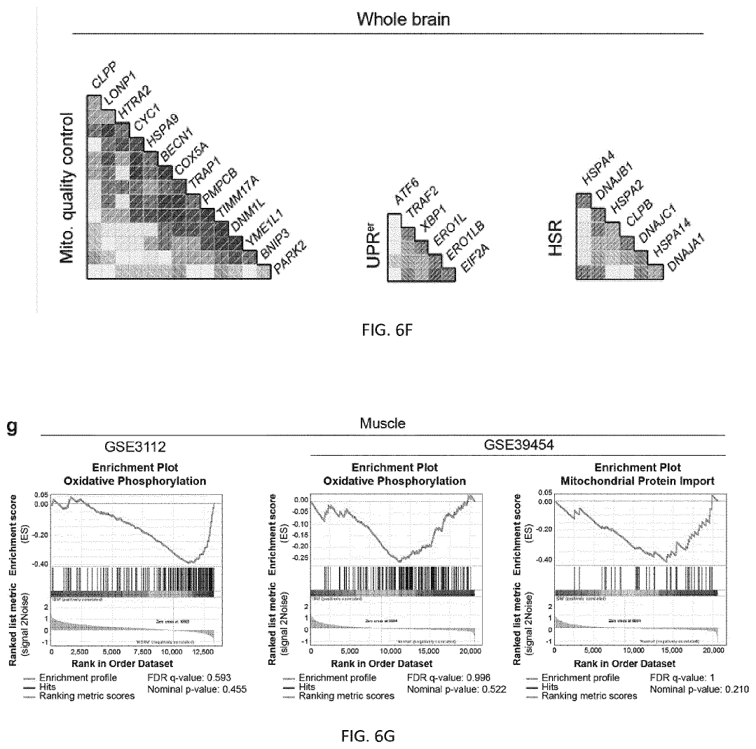

We investigated brain expression datasets from AD patients (GN327, GN328 and GN314) archived in the GeneNetwork web resource (www.genenetwork.org) to define the mitochondrial signature associated with the disease. Gene Set Enrichment Analysis (GSEA) of datasets from healthy versus AD individuals in prefrontal, primary visual cortex and whole brain showed that downregulation of mitochondrial oxidative phosphorylation (Oxphos) and perturbation of mitochondrial import pathways were hallmarks of AD (FIGS. 1A-1B, FIGS. 6A, 6B, 6D, 6E). As these processes are tightly linked with, and affect mitochondrial proteostasis, we used comprehensive gene sets for two major mitochondrial quality control pathways, the mitochondrial unfolded protein response (UPR^{mt}) and mitophagy, to evaluate whether the expression of these genes is co-regulated in AD patients. Whereas we observed a tight correlation between genes typifying UPR^{mt} and mitophagy in all brain datasets investigated (FIG. 1C and FIGS. 6C and 6F), other stress pathways, such as the ER stress (UPR^{er}) and heat shock response (HSR) were co-regulated but to a lower degree (FIG. 1C and FIGS. 6C and 6F). In line with the results in AD, GSEA of muscle expression datasets from healthy and IBM individuals (GSE3112 and GSE39454) showed an overall tendency towards down-regulation of genes key to mitochondrial function (FIG. 6G).

Experimental evidence for a mitochondrial stress signature in AD.

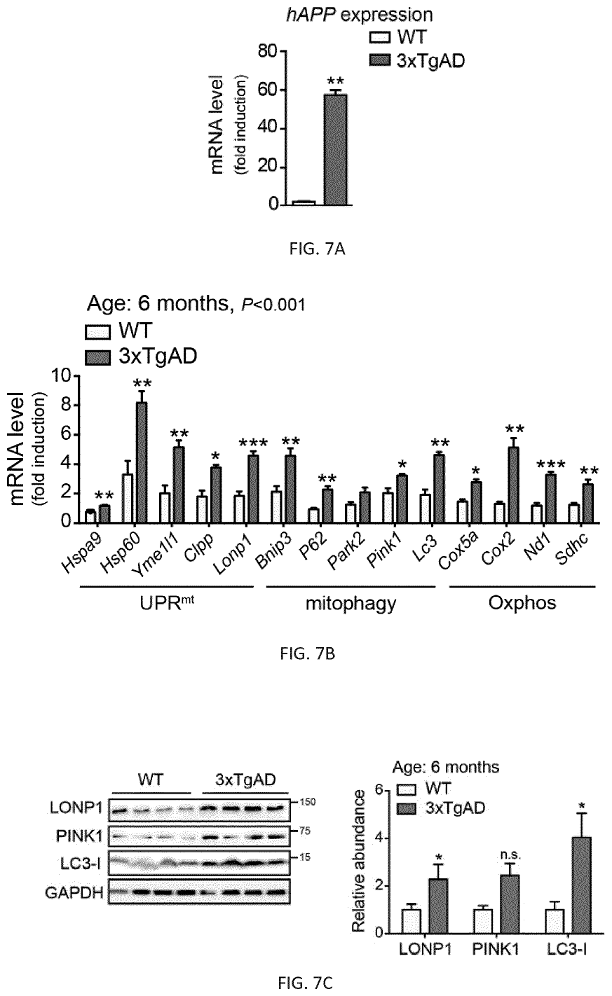

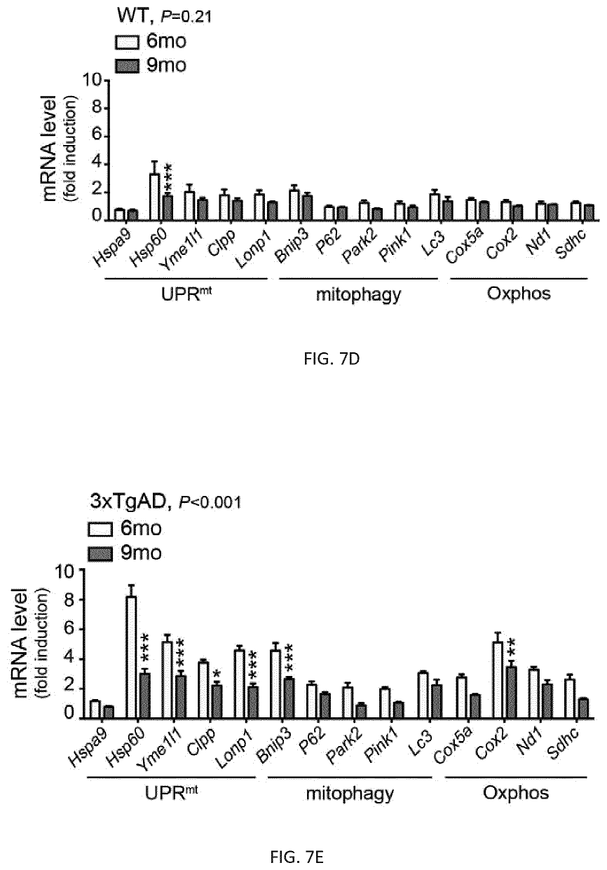

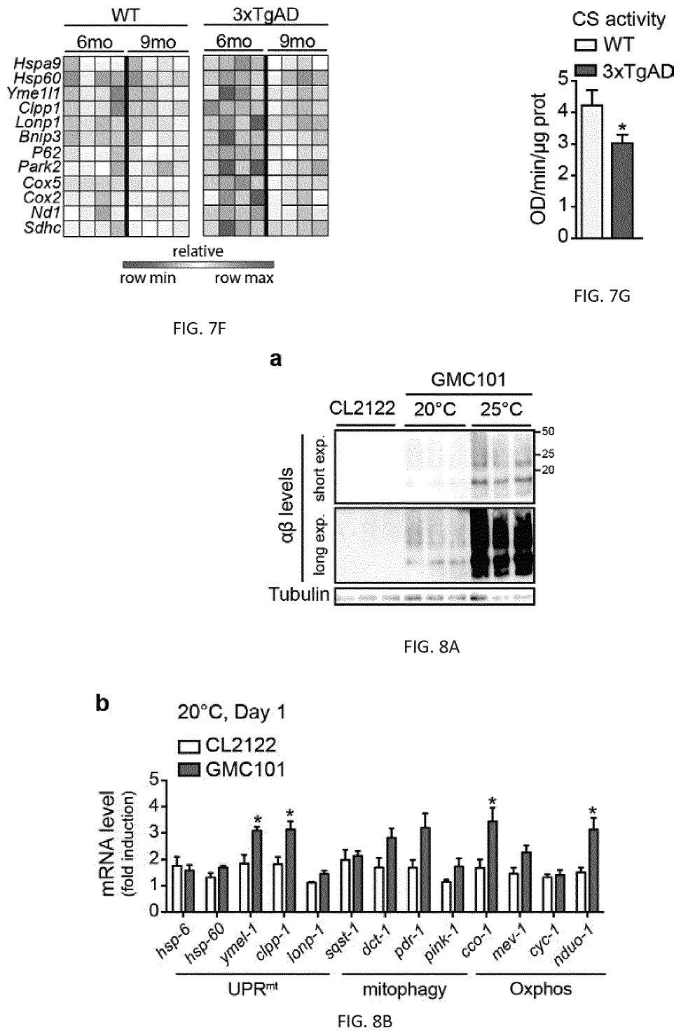

We then measured the levels of UPR^{mt}, mitophagy and Oxphos transcripts in cortex samples of humans. Previously, we have reported that several UPR^{mt} genes were up-regulated during frank familial or sporadic AD. Here, we extended that analysis and observed that, compared to subjects with no cognitive impairment (NCI), several UPR^{mt} and mitophagy transcripts were also up-regulated in patients with mild cognitive impairment (MCI), a putative prodromal AD stage, and in mild/moderate AD (FIG. 1D), whereas Oxphos genes were down-regulated, consistent with our GSEA. The induction of this mitochondrial stress response was also observed at the protein level in MCI and AD subjects (FIG. 1E). The occurrence of this perturbation already in MCI suggests that mitochondrial dysfunction contributes to neuron and synapse loss, and that mitochondrial stress pathways may be activated as a protective response during disease progression. We also analyzed cortex samples of wild-type (WT) and 3×TgAD mice (FIG. 7A) at two different ages, i.e. 6 and 9 months. Both mitochondrial quality control pathways and Oxphos genes were induced in AD mice (FIG. 1F and FIG. 7B), although to a different extent over time. In fact, pairing WT and AD animals at these two time points indicated a marked attenuation of this stress signature during disease progression (FIGS. 7D-7F). Immunoblotting of total lysates from the cortex of WT and 3×TgAD mice showed the induction of PINK1, LONP1 and LC3 at both time points (FIG. 1G and FIG. 7C). Additional analysis of the 9-month old animals also indicated a reduction in VDAC, a marked increase in P62 phosphorylation (FIG. 1G), and reduced citrate synthase (CS) activity (FIG. 7G) in AD mice, indicative of activated autophagy and mitophagy. PINK1 and LC3-I were also increased upon immunoblotting of mitochondrial extracts from cortex samples of the AD mice (FIG. 1H), confirming that these proteins are recruited to the mitochondria to promote mitophagy, as supported by the augmented ubiquitylation of mitochondrial proteins (FIG. 1H). For simplicity, we abbreviate the comprehensive mitochondrial stress footprint analyzed herein as Mitochondrial Stress Response (MSR).

Identification of a Cross-Species MSR Signature

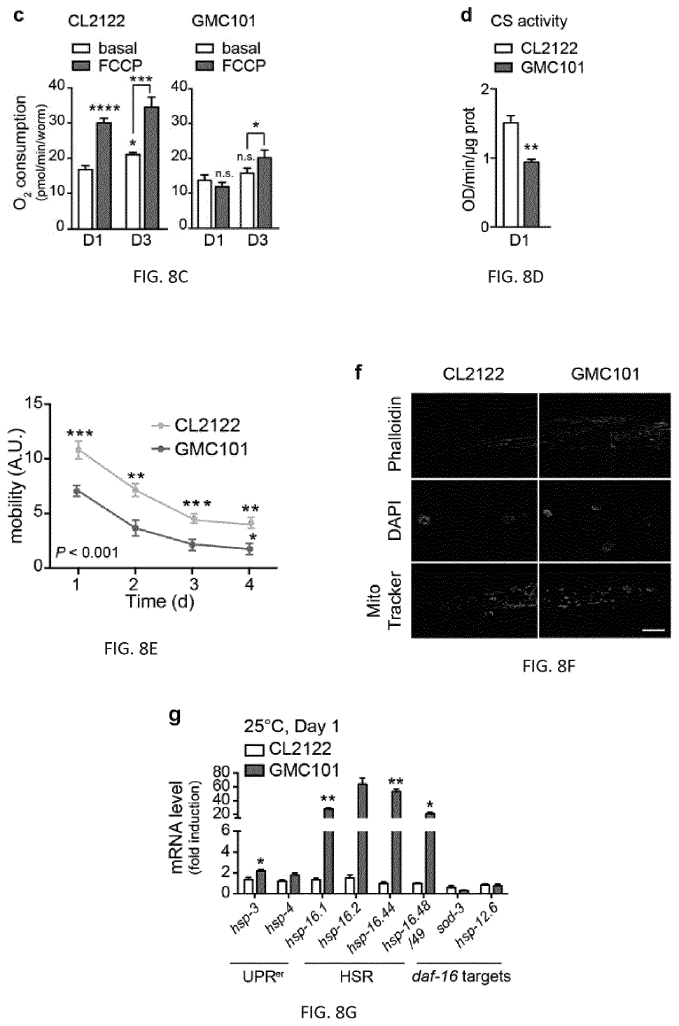

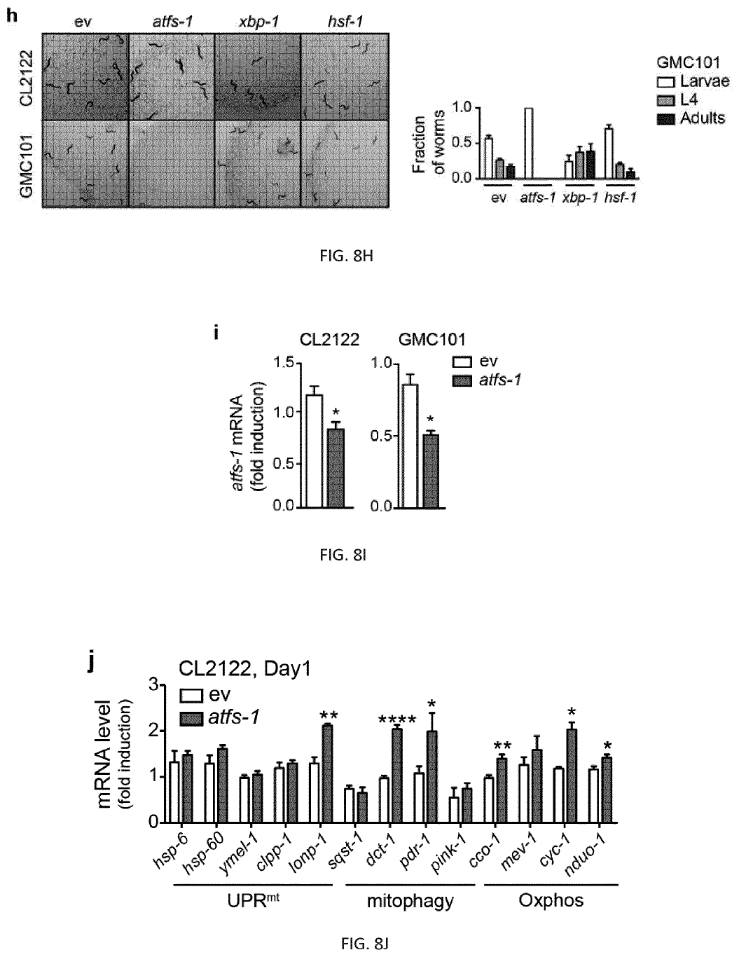

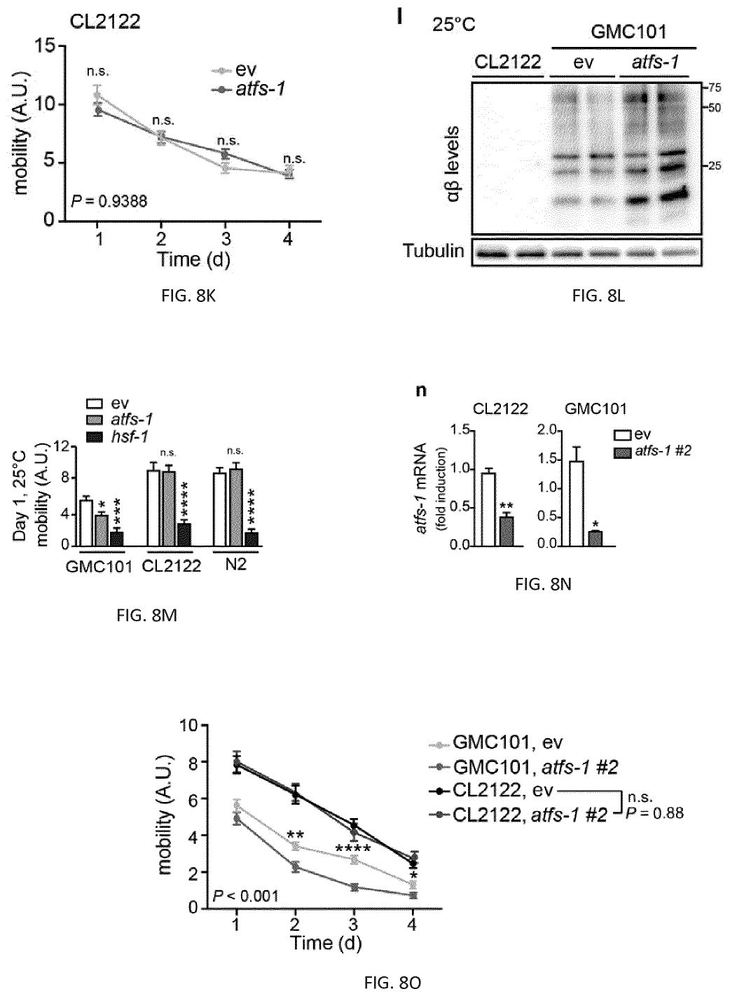

The functional impact of changes in mitochondrial homeostasis during disease and aging in mammals can be rather faithfully translated in the nematode Caenorhabditis elegans. Worm models of Aβ aggregation have been extensively used to study the basic consequences of proteotoxic stress on conserved biological pathways between worms and mammals, and to screen and validate compounds affecting these processes. Hence, we took advantage of the GMC101 worm model of Aβ proteotoxicity. GMC101 worms constantly express the human Aβ isoform 1-42 in muscle cells, but adults only develop age-progressive paralysis and amyloid deposition in the body wall muscle after a temperature shift from 20 to 25° C., while the control strain CL2122 does not express the Aβ peptide (FIG. 8A). Given the muscle-targeted overexpression of the Aβ peptide in GMC101, one should consider this strain as a general model of Aβ disease, and equally suitable to mimic the proteotoxic phenotypes observed in AD and IBM.

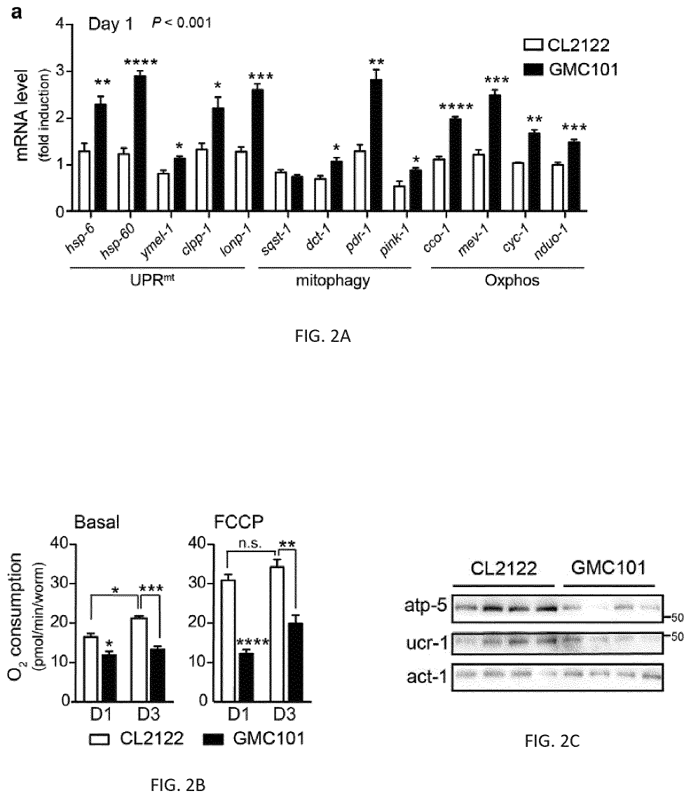

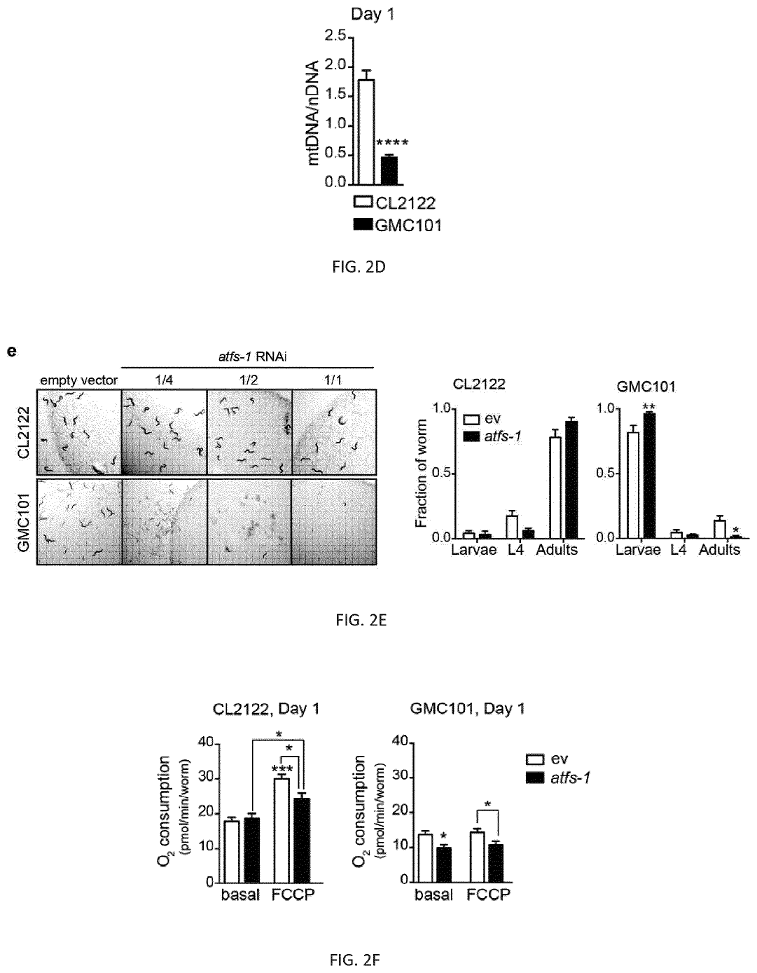

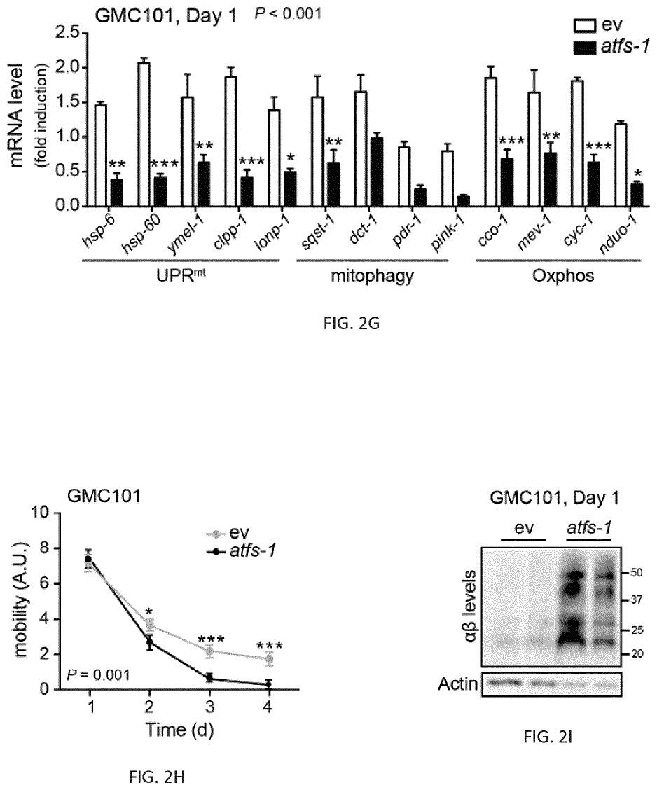

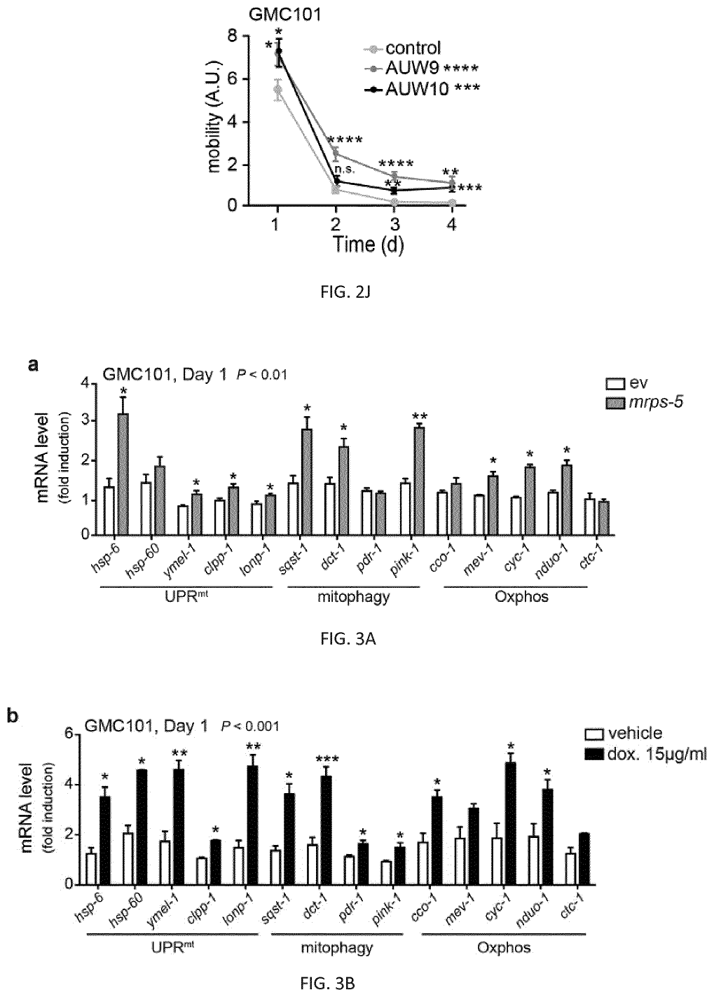

Transcripts of the worm orthologs of the MSR were induced in adult GMC101 worms compared to CL2122 following the temperature shift (FIG. 2A), while only partially perturbed at 20° C. (FIG. 8B). Basal and maximal respiratory capacity were decreased in GMC101 (FIG. 2B and FIG. 8C), in contrast with the increased Oxphos transcript levels (FIG. 2A), suggesting a compensatory induction of these genes to ensure respiration. Mitochondrial content was lower in GMC101 worms, as shown by decreased Oxphos proteins, mt/nDNA ratio and CS activity (FIGS. 2C-2E). Importantly, GMC101 fitness, measured as spontaneous movement, was robustly reduced relative to CL2122 in line with the evident muscle disorganization and alteration of the mitochondrial network in the body wall muscle (FIGS. 8D and 8E). These data highlight the cross-species conservation of the MSR, and make GMC101 an excellent proxy to characterize the mitochondrial dysfunction and phenotypic impact observed in Aβ diseases in mammals.

Mitochondrial homeostasis protects against proteotoxic Aβ disease.

The control of mitochondrial function and UPR^{mt} during stress in the worm is largely attributable to the activating transcription factor associated with stress, atfs-1. Strikingly, depletion of atfs-1 by RNAi feeding of the GMC101 worms, but not CL2122, caused a severe developmental delay even in absence of the “disease-inducing” temperature shift (FIG. 2F), phenocopying mitochondrial respiration mutants that rely on atfs-1 for survival and adaption. Comparative transcript analysis of genes involved in cytosolic and nuclear adaptation pathways, such as UPR^{er}, HSR, and daf-16, between GMC101 and CL2122 showed also a mild induction of the UPR^{er} and a striking upregulation of the HSR genes in GMC101 (FIG. 8F), in line with the role of HSR as a primary defense against proteotoxic stress in worm. We therefore evaluated the effect of RNAis targeting key regulators of these pathways, hsf-1 and xbp-1, on the development of the GMC101 strain. Only atfs-1 RNAi again led to extreme developmental delays, while no alterations were observed with all these RNAis in CL2122 (FIG. 8G).

Importantly, basal and maximal respiration in adult GMC101 worms was significantly impaired upon atfs-1 silencing, while only maximal respiration was partially affected in CL2122 (FIG. 2G). Furthermore, atfs-1 knockdown in GMC101 resulted in a prominent repression of the overall MSR signature, including mitophagy effectors (FIG. 2H). Conversely, in CL2122, pdr-1, dct-1 and Oxphos transcripts were even induced, in spite of atfs-1 silencing in both strains (FIGS. 8H-8I). In addition, upon atfs-1 RNAi in GMC101 worms, paralysis was exacerbated (FIG. 2I), while CL2122's mobility was unaffected (FIG. 8J), and the accumulation of amyloid aggregates was increased (FIG. 2J and FIG. 8K).

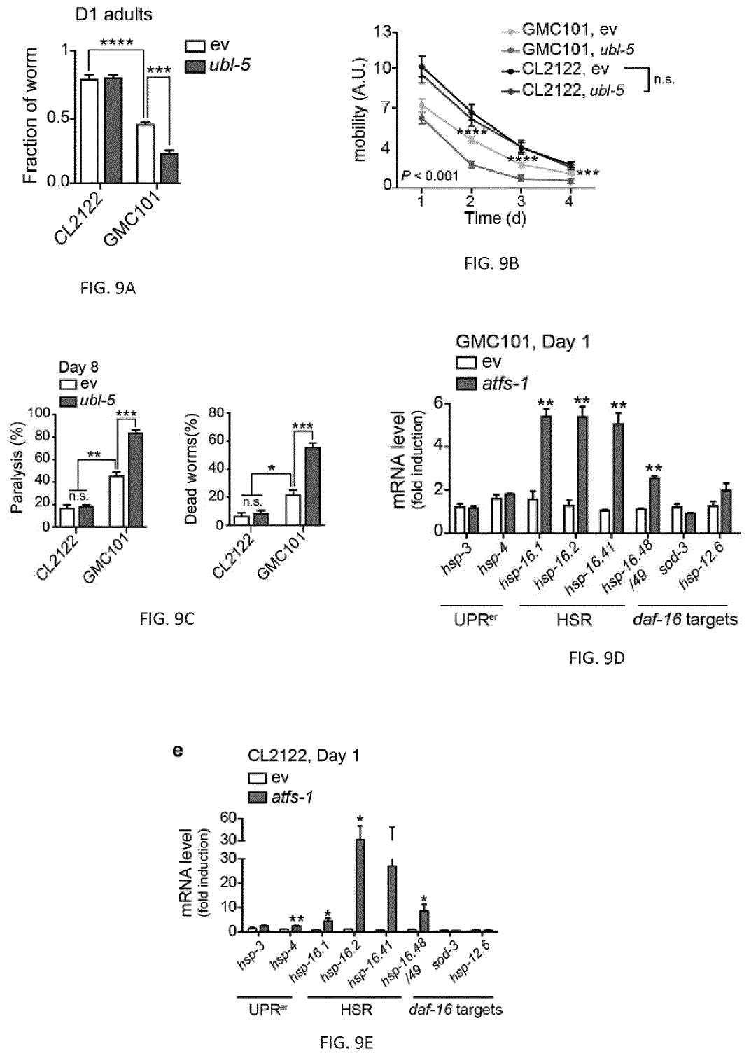

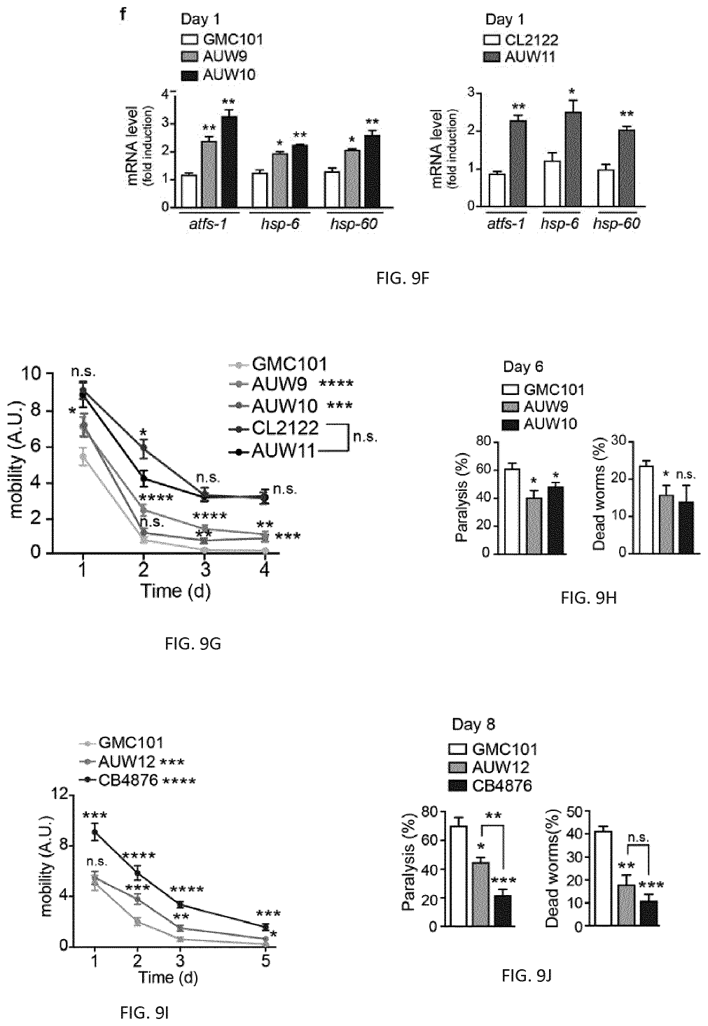

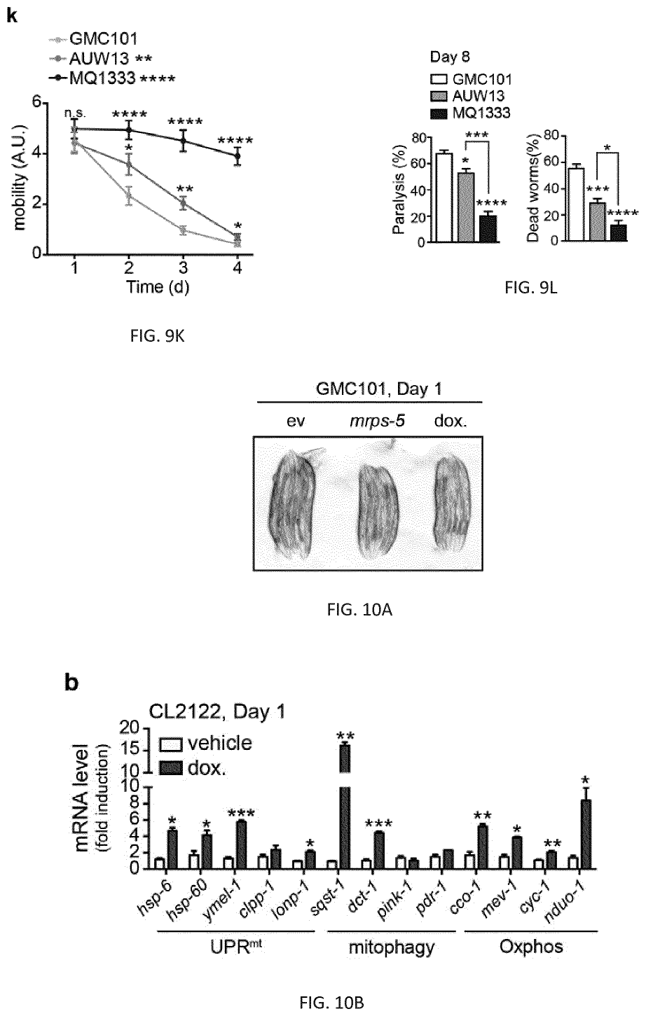

Given the induction of the HSR in the GMC101, we tested whether hsf-1 repression would impact on worms' fitness. Interestingly, while atfs-1 knockdown only paralyzed GMC101 worms, silencing of hsf-1 reduced mobility in both CL2122 and wild-type N2 strains when incubated at 25° C. (FIG. 8L), reflecting a general effect of hsf-1 on homeostasis independent of the strain used. Worm mobility was similarly impaired in GMC101, but not in CL2122, following efficient silencing of atfs-1 with an alternative RNAi we generated (atfs-1 #2) (FIGS. 8M-8N), confirming a specific role of atfs-1 in ensuring organismal homeostasis in GMC101 worms. Furthermore, silencing of ubl-5, another positive regulator of the nematode UPR^{mt}, also delayed development and decreased health- and lifespan specifically in GMC101 (FIGS. 9A-9C). Intriguingly, in addition to perturbation of mitochondrial pathways, atfs-1 silencing led to further upregulation of the HSR signature in GMC101 and to its induction in CL2122 (FIGS. 9D-9E), while repressing the mitochondrial stress signature specifically in GMC101 (FIG. 2G and FIG. 8I).

Conversely, to enhance atfs-1 function, we generated two GMC101-derived strains, AUW9 and AUW10, and one CL2122 line (AUW11), overexpressing atfs-1. This resulted in the induction of the UPR^{mt} (FIG. 9F), a significant increase in fitness, and a discrete decrease in paralysis and death scores in the GMC101-derived strains AUW9 and AUW11 (FIG. 2K and FIGS. 9G-9H), while no changes were observed in AUW11 (FIG. 9G). As an alternative approach to increase mitochondrial stress response, we crossed the GMC101 strain with two long-lived mitochondrial mutants, i.e. clk-1 and nuo-6. Consistently, GMC101 with a mutation in these mitochondrial genes (AUW12 and AUW13) manifested intermediate phenotypes between the GMC101 and their mitochondrial mutant counterpart, with enhanced healthspan and lifespan (FIGS. 9I-9L).

Altogether, this indicates that atfs-1 and the MSR induction are essential to ensure proteostasis and survival in this worm model of Aβ aggregation, and that mitochondria play an active, rather than passive, role during Aβ proteotoxic stress. This prompted us to investigate the potential of boosting mitochondrial proteostasis and function to curb the progression of this deleterious phenotype.

Interfering with mitochondrial translation reduces Aβ proteotoxicity through the UPR^{mt} and mitophagy.

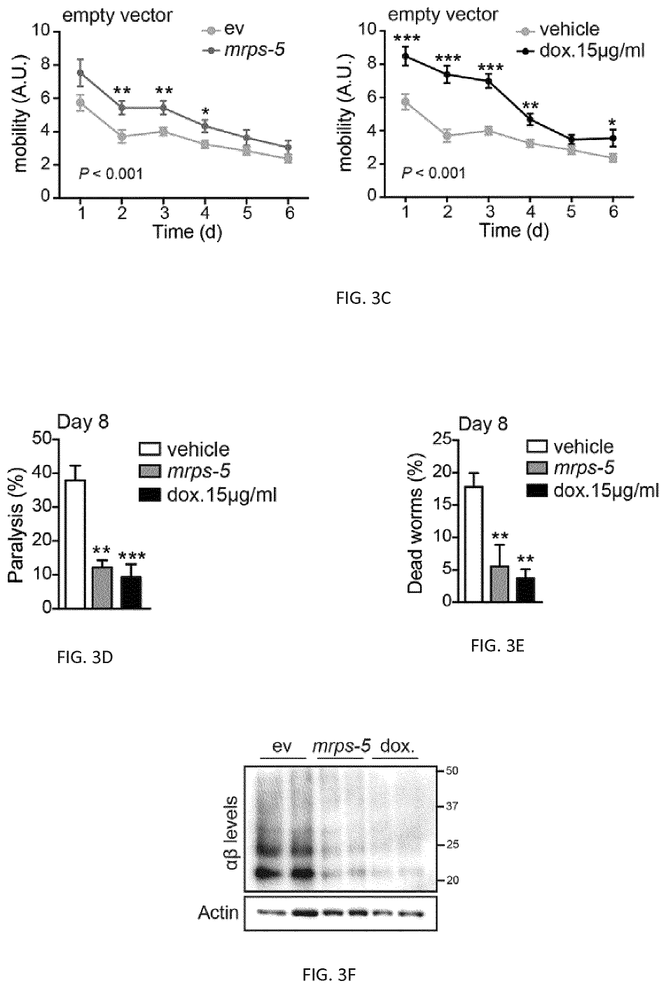

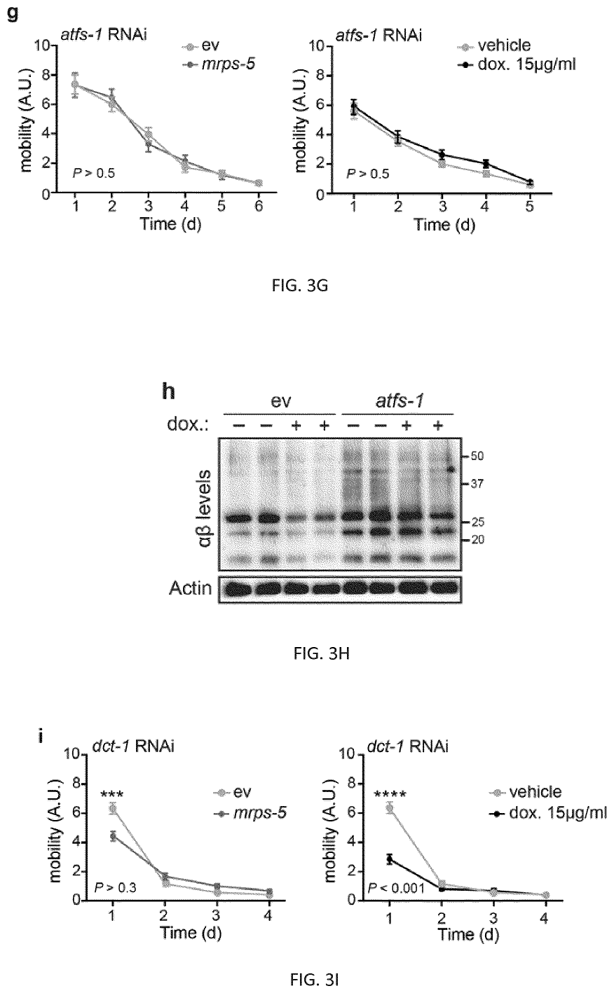

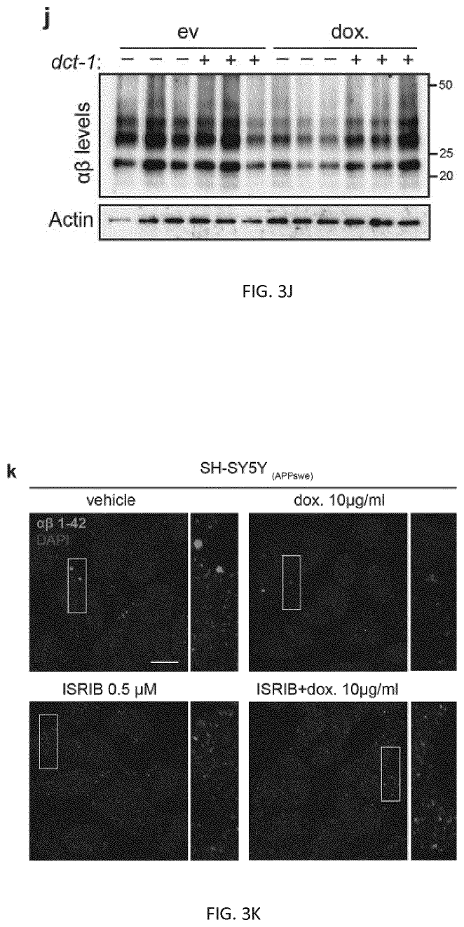

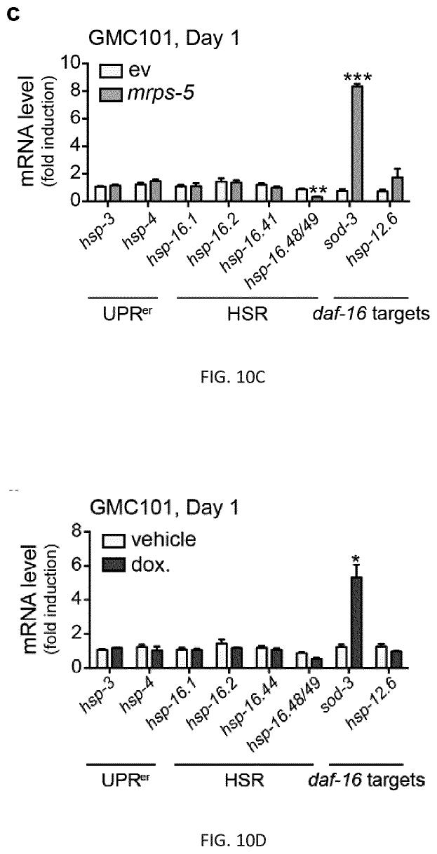

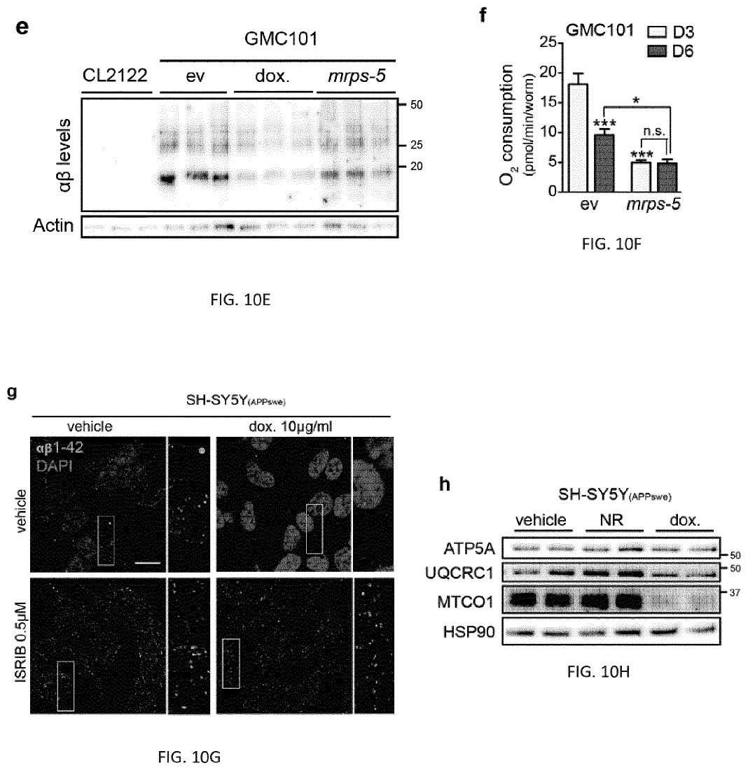

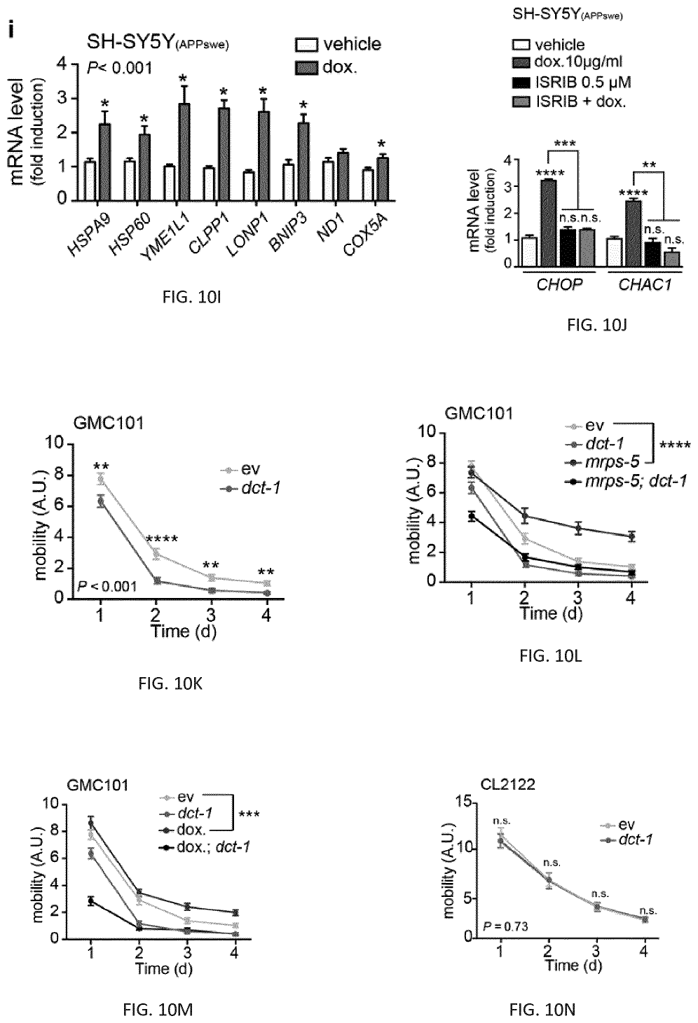

Given the tight link between the UPR^{mt} and AD observed above, we investigated the effects of two established strategies to induce the UPR^{mt} in C. elegans; genetically, by silencing the expression of the mitochondrial ribosomal protein mrps-5, and pharmacologically, by using the mitochondrial translation inhibitor doxycycline (dox). Both these interventions, which are known to favor worm health and lifespan, markedly induced the UPR^{mt} transcripts and increased the expression of mitophagy and respiration genes in GMC101 (FIGS. 3A-3B), without causing major development and growth delays (FIG. 10A). Dox similarly induced these pathways in CL2122 (FIG. 10B). Transcript analysis of the other stress pathways revealed no perturbations of UPR^{er} and HSR in GMC101 treated with dox or mrps-5 RNAi (FIGS. 10C-10D), while a consistent induction of one daf-16 target, the mitochondrial superoxide dismutase sod-3, was observed (FIGS. 10C-10D). The transcriptional induction of the MSR impacted beneficially on fitness and lifespan of GMC101 worms (FIGS. 3C-3E). Furthermore, Aβ aggregation was reduced by mrps-5 RNAi and dox (FIG. 3F and FIG. 10E). Of essence, the improvement in motility and Aβ clearance in GMC101 required atfs-1 (FIGS. 3G-3H), proving the vital contribution of the UPR^{mt} to the phenotypic improvements. Interestingly, respirometry on GMC101 worms fed with mrps-5 RNAi at D3 and D6 of adulthood (FIG. 10F), showed that mrps-5 knockdown prevented the decrease in respiration during aging in this strain, suggesting a stabilization of mitochondrial function following the MSR-dependent improvement of proteostasis.

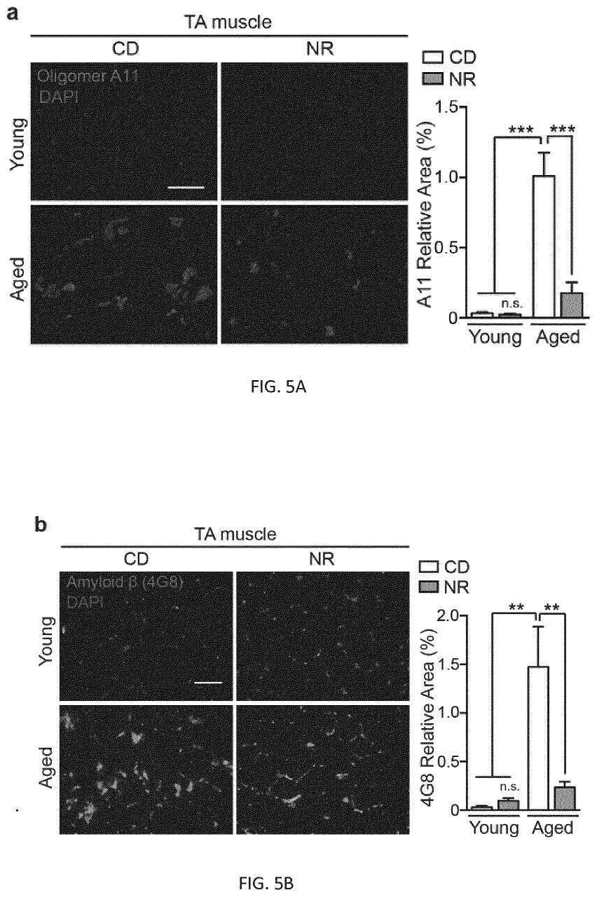

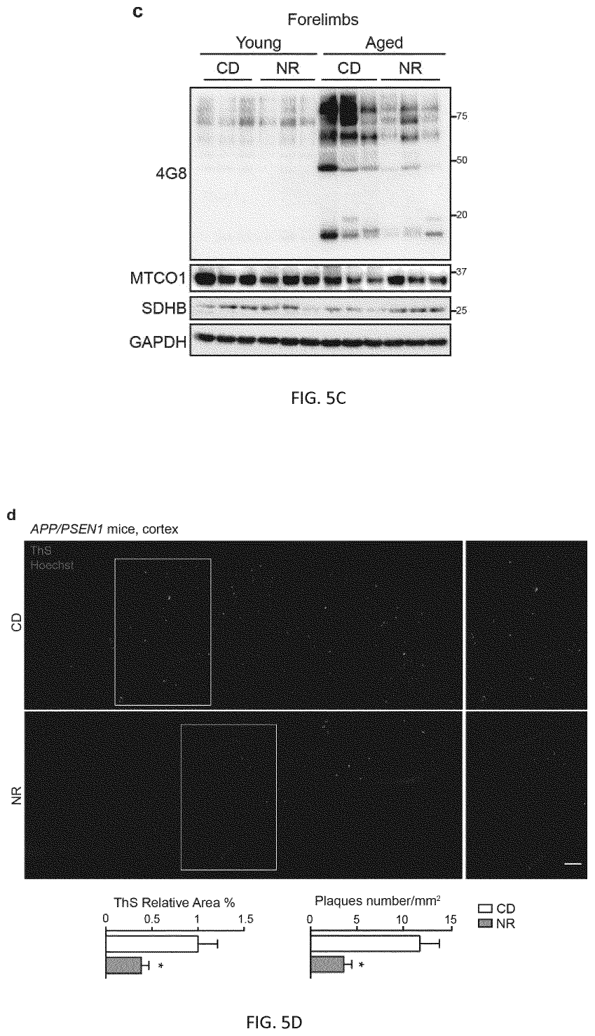

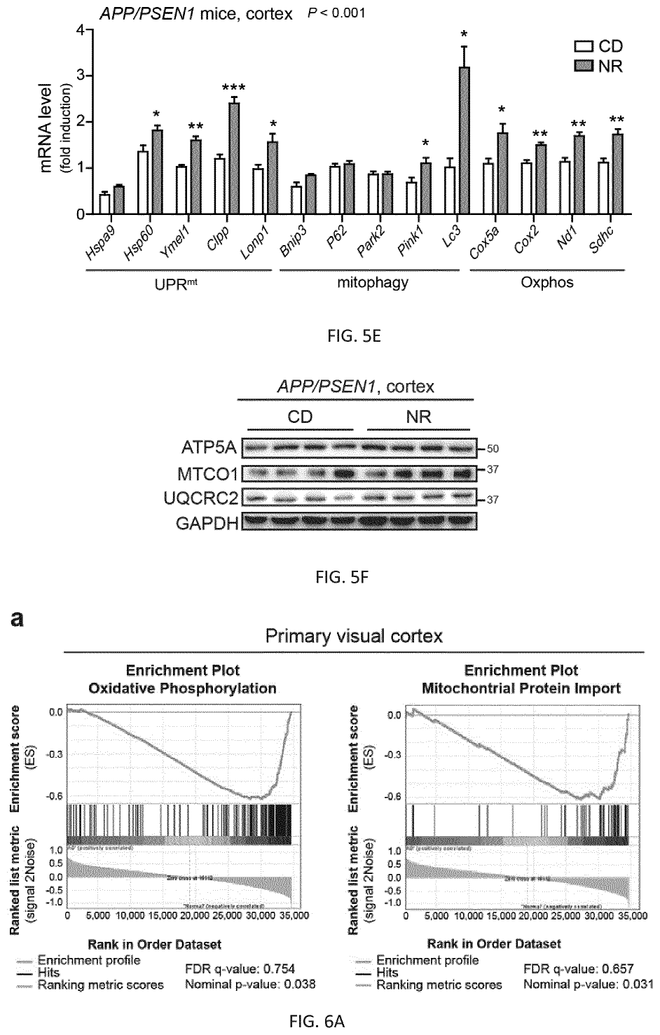

We then extended our investigation to a mammalian system using the SY5Y neuroblastoma cell line expressing the APP Swedish K670N/M671L mutation (APP_{Swe}). Dox markedly reduced intracellular Aβ deposits, as shown by immunodetection with an Aβ1-42-specific antibody (FIG. 3K and FIG. 10G). This improvement was linked to a mito-nuclear protein imbalance and the induction of components of the MSR transcript signature (FIGS. 10H-10I). These results add to observations of in vitro and in vivo studies in AD flies and patients, which suggested that dox treatment may ameliorate AD and Aβ aggregation. Recently, the regulation of the mitochondrial stress responses in mammals, including dox-dependent mitochondrial stress, was shown to rely on the transcription factor ATF4. Pretreating cells with ISRIB, a global inhibitor of the integrated stress response (ISR) that blocks the activity of the translation initiation factor eIF2α, thereby inhibiting ATF4 translation, prevented amyloid clearance by dox and hampered the dox-mediated induction of canonical ATF4 target genes, such as CHOP and CHAC1 (FIG. 3K and FIGS. 5G and 5J). This indicates the involvement of ATF4-dependent pathways in resolving Aβ proteotoxic stress in mammalian cells.

Given the induction of mitophagy in 3×Tg mice, human AD patients and in the GMC101 worms, and its further increase upon mrps-5 RNAi and dox treatments, we also tested the contribution of mitophagy to the homeostasis of GMC101. To achieve this, we silenced by RNAi dct-1, an evolutionarily conserved key regulator of mitophagy. dct-1 RNAi reduced GMC101's health- and life-span already in basal conditions (FIGS. 10K-10M). Furthermore, it also blunted the positive effects of mrps-5 RNAi and dox on health- and lifespan (FIG. 3I and FIGS. 10L and 10M) and on proteostasis (FIG. 3J). Conversely, dct-1 knockdown in CL2122 worms affected their movement only during aging (FIGS. 10N and 10O), stressing the relevance of mitophagy as a key process in aging. Altogether, these data show that mitophagy, in addition to UPR^{mt}, is also induced and required for the survival of GMC101 worms and for the beneficial effects of the described interventions.

NAD⁺ boosters attenuate Aβ proteotoxicity through the UPR^{mt} and mitophagy.

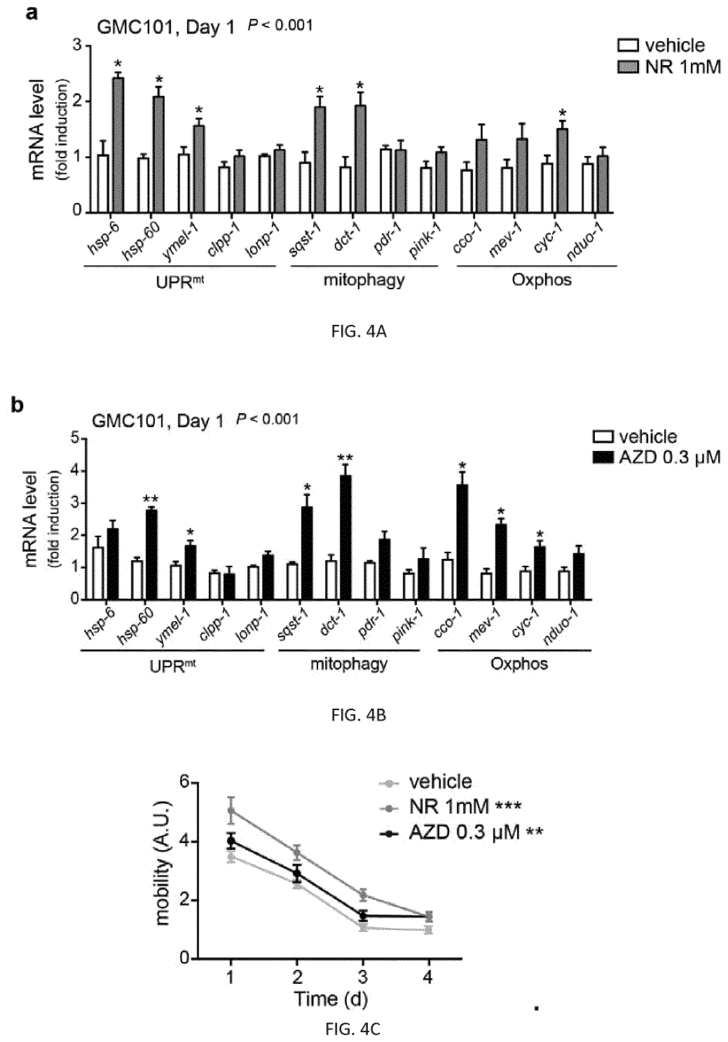

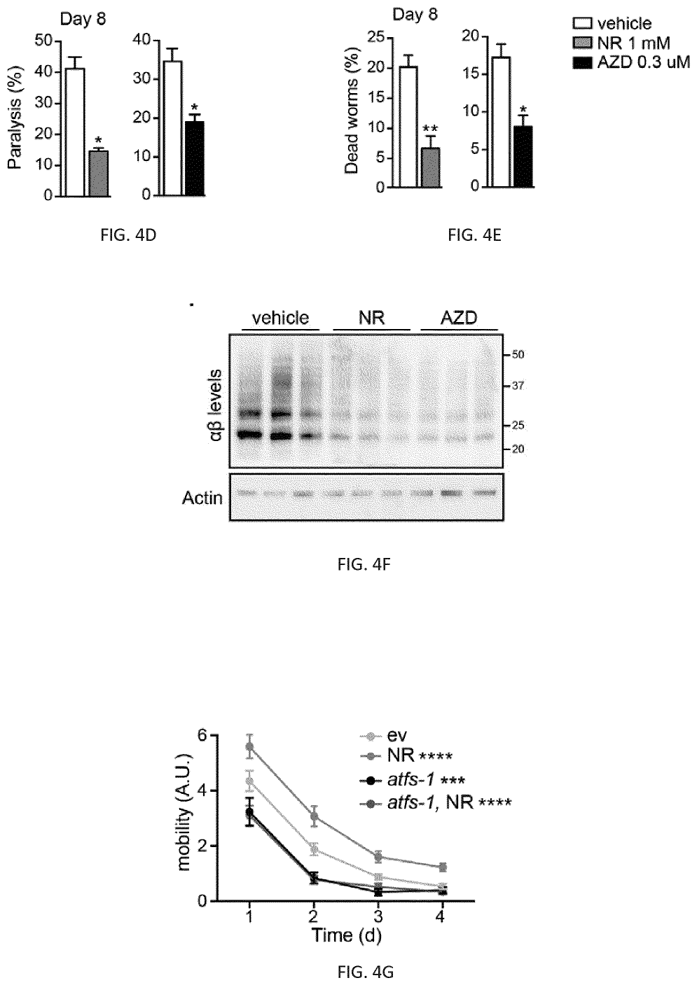

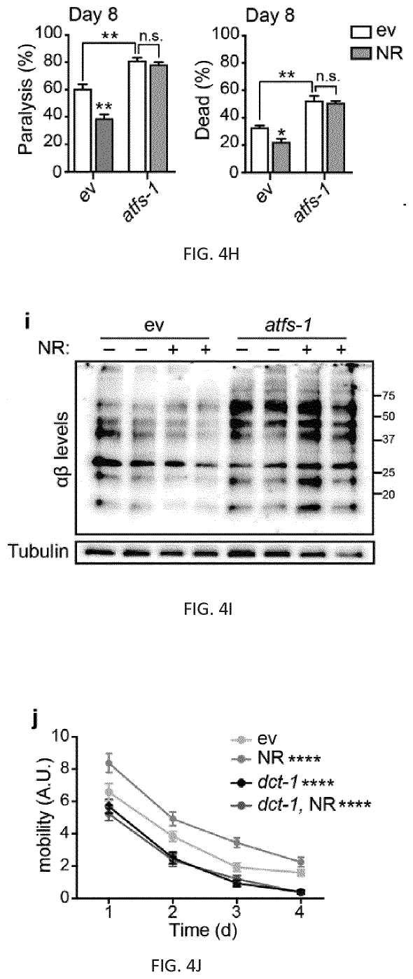

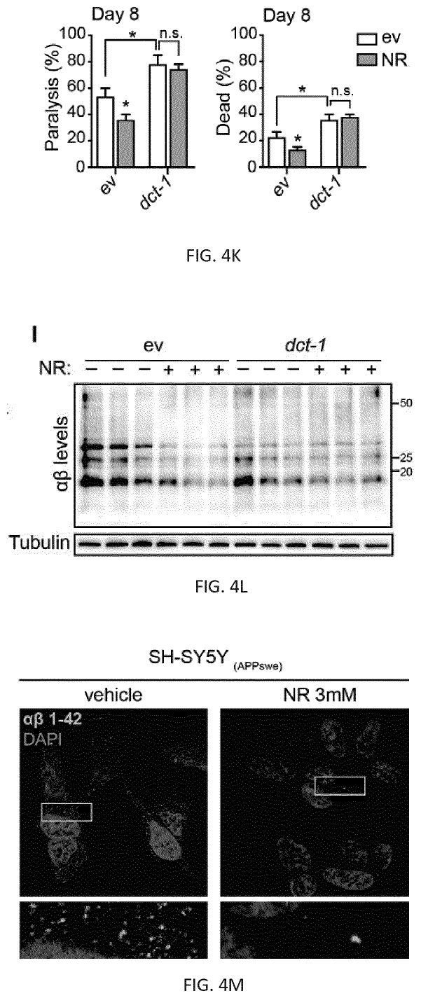

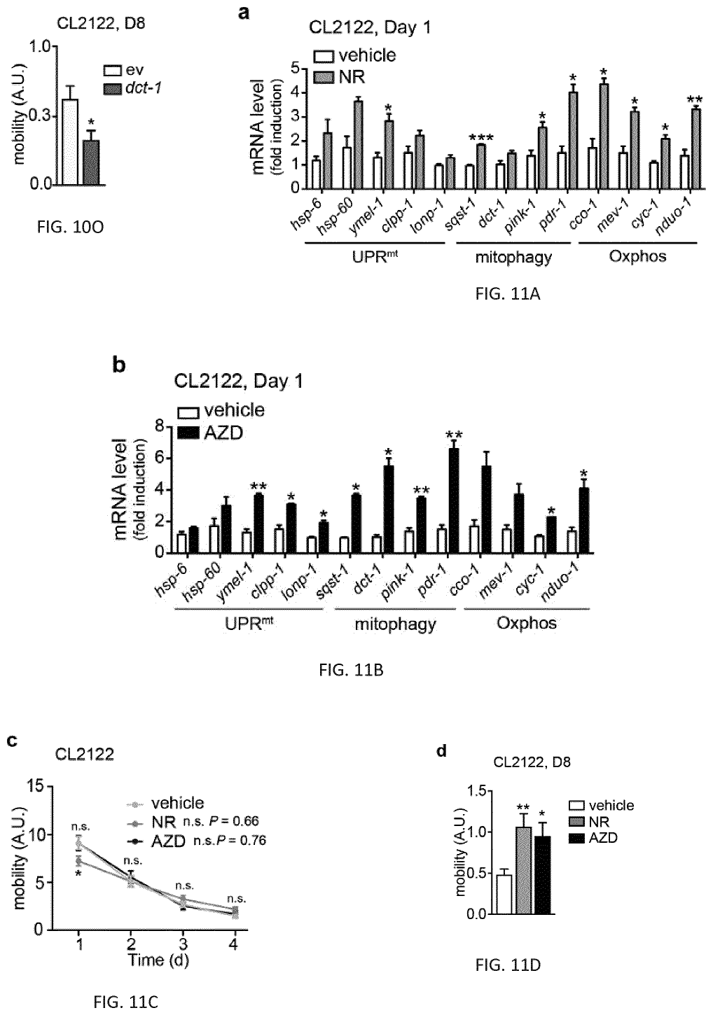

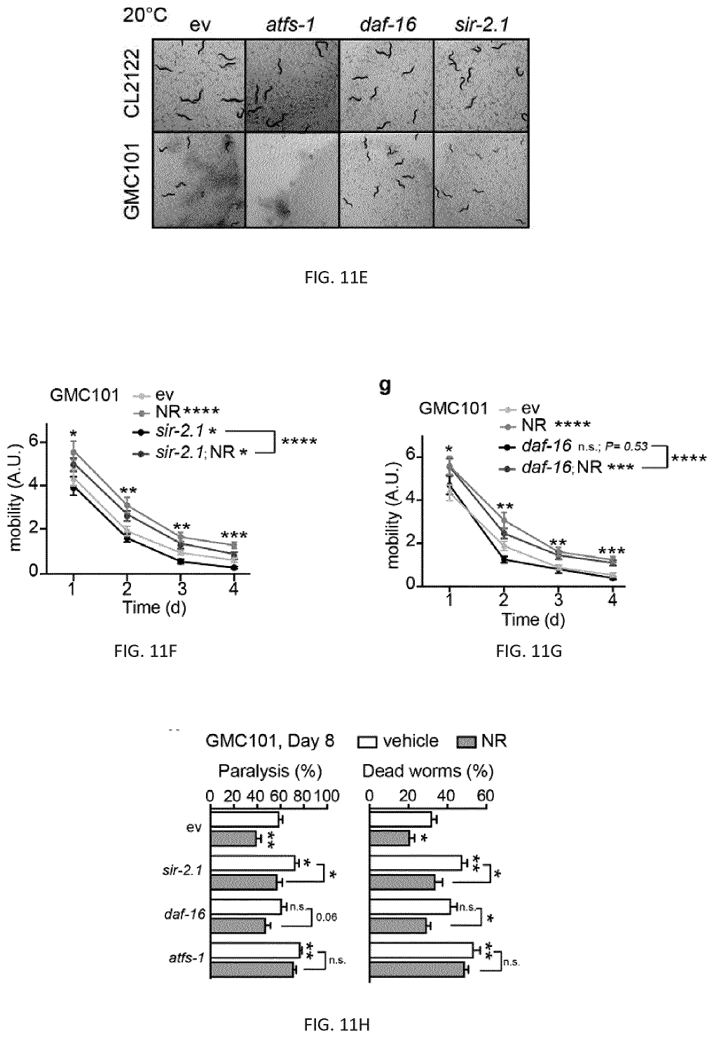

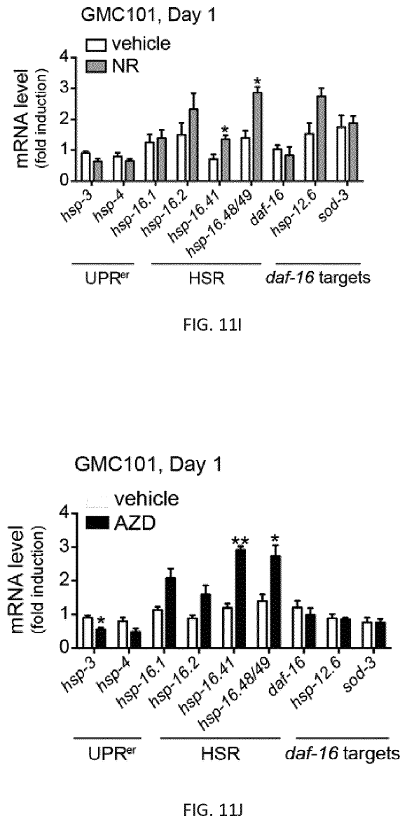

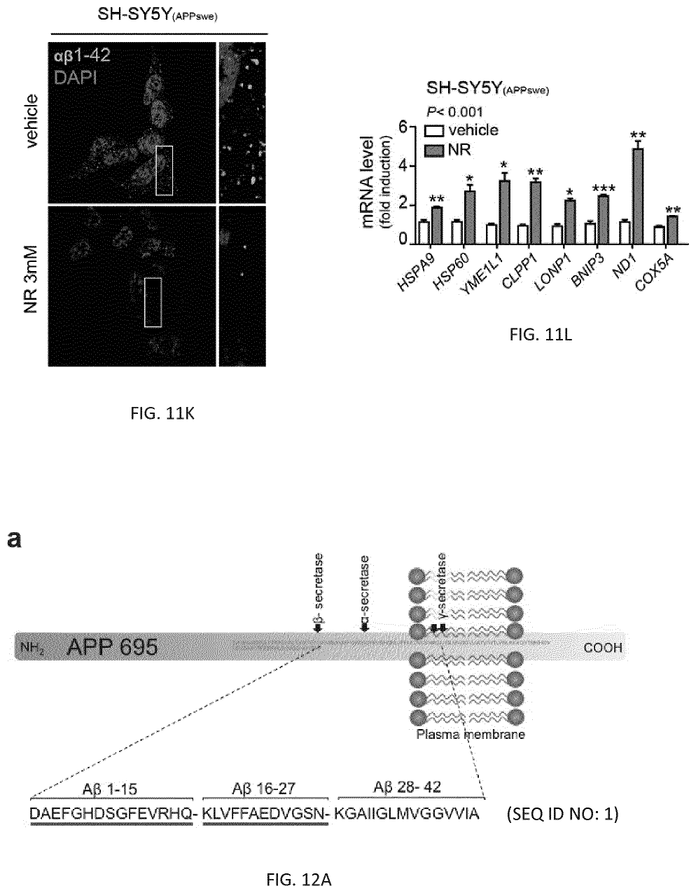

The UPR^{mt} and mitophagy pathways are also potently induced in worms and in various mammalian tissues by NAD⁺-boosting compounds, such as nicotinamide riboside (NR), and Olaparib (AZD2281 or AZD). Similarly to dox intervention and mrps-5 RNAi, treatment of GMC101 with NR and AZD induced the MSR (FIGS. 4A-4B), and improved healthspan and lifespan (FIGS. 4C-4E). The NR- and AZD-mediated induction of the MSR was also consistently observed in CL2122 (FIGS. 11A-11B), but, these treatments only improved CL2122 fitness during aging (FIGS. 11C-11D), similarly to what previously shown in N2 worms. In addition, treatment of GMC101 with NR and AZD reduced proteotoxic stress (FIG. 4F). Importantly, the NR-mediated phenotypic and proteostasis benefits required atfs-1 (FIGS. 4G-4I) and dct-1 (FIGS. 4J-4L). NR has been shown to increase sirtuin activity, and activate the FOXO/daf-16 signaling in mammals and in C. elegans. Therefore, we evaluated the effect of daf-16 and sir-2.1 silencing on development, healthspan and NR-dependent benefits of GMC101. While sir-2.1 knockdown did not delay the growth of GMC101 or its control (FIG. 11E), it increased paralysis and death in the GMC101 worms similarly to dct-1 and atfs-1 RNAis (FIGS. 11F and 11H). However, NR still significantly rescued health- and life-span of GMC101 fed with sir-2.1, showing that the positive effects of NR rely mostly on atfs-1 and dct-1 in these worms (FIGS. 11F and 11H). Instead, feeding GMC101 with daf-16 RNAi did not result in any major phenotypic changes (FIGS. 11E, 11G, and 11H). Furthermore, NR did not affect the expression levels of daf-16 and its targets (FIG. 11I). NR and AZD had only a minimal impact on the expression UPR^{er} and HSR genes in GMC101, with induction of hsp-16.41 and hsp-16.48/49 (FIGS. 11I-11J). These results indicate that in the GMC101 model, the main mode of action of NAD⁺ boosting involves the induction of the MSR.

We also assessed the effect of NR in the Aβ-expressing neuronal cells and consistent with the data in C. elegans, we observed a remarkable reduction of the intracellular Aβ deposits with NR (FIG. 4M and FIG. 11K), accompanied by increased Oxphos protein (FIG. 10H) and MSR transcript levels (FIG. 11L).

NR reduces Aβ levels in AD transgenic mice and protein aggregation in aged mice.

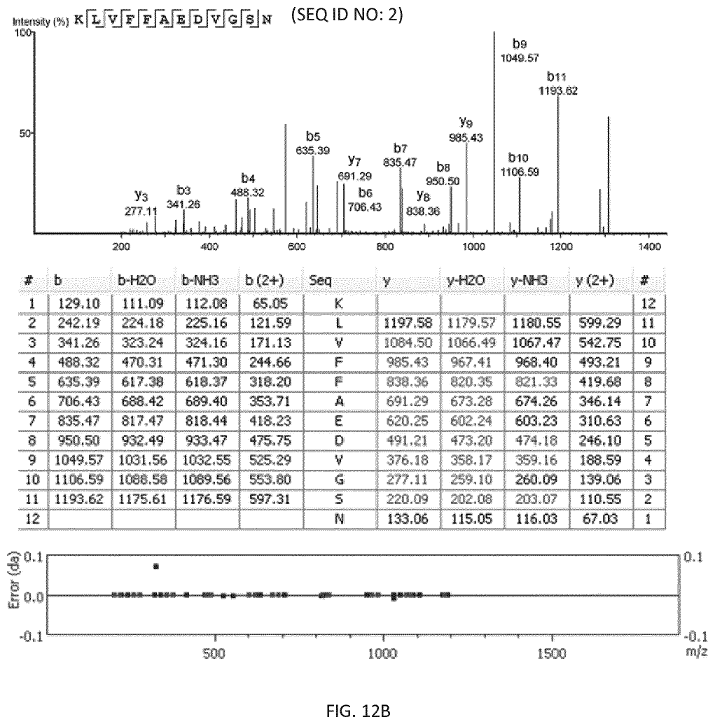

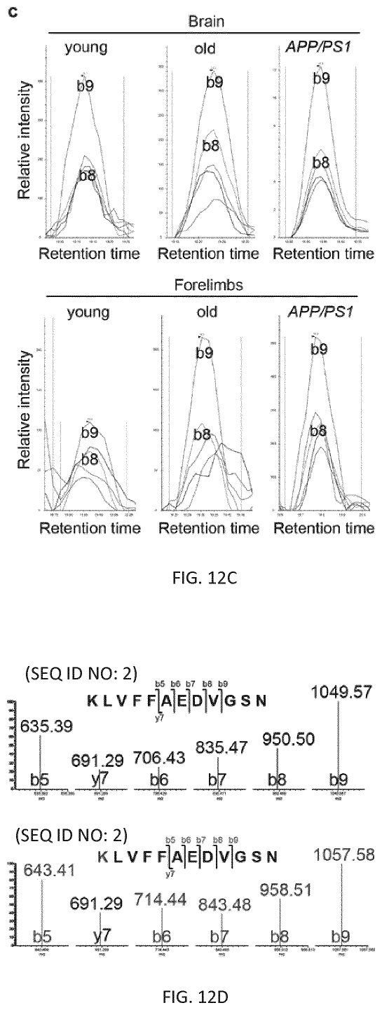

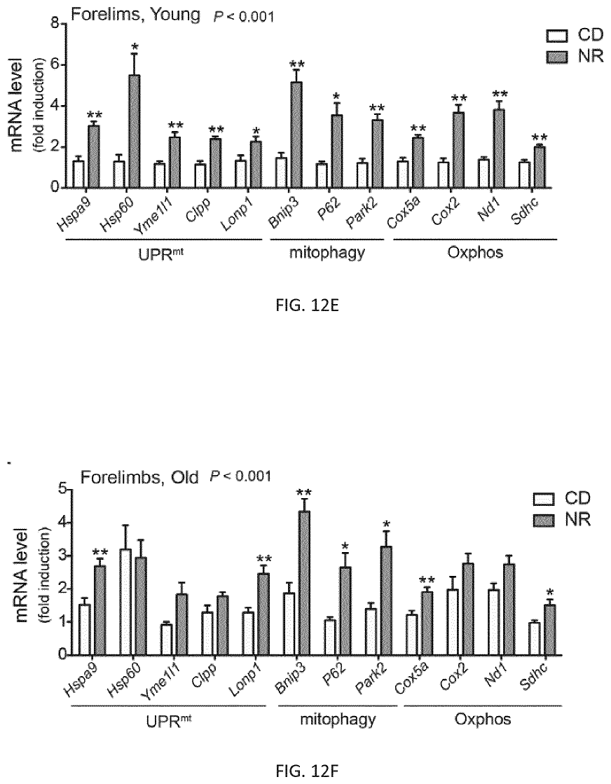

Aging is accompanied by a concomitant decrease in mitochondrial function and proteostasis. Whether natural aging in mice is linked to increased formation of protein aggregates of amyloid nature in muscle tissues is, however, unclear. We therefore assessed the levels of amyloid-like aggregates in tibialis anterior (TA) and forelimbs muscle tissues from young (˜3 months) and old (˜24 months) C57BL/6J mice, using anti-Aβ (4G8) and anti-oligomer (A11) antibodies. Intriguingly, we found that both muscles from aged mice present high levels of protein aggregates, suggestive of age-related amyloidosis (FIGS. 5A-5C). By performing mass spectrometry coupled with liquid chromatography (LC-MS/MS) together with multiple reaction monitoring (MRM) of 4G8-immunopreciptates from muscles and brains from young, old and transgenic APP/PSI AD mice (FIGS. 12A-12D) we could unambiguously detect the presence of endogenous Aβ peptide in these tissues; these data support the presence of Aβ amyloid aggregates under the 4G8-dependent signals in muscle tissues of aged animals. In addition, the protein levels of SDHB and MTCO1, representative Oxphos components, were downregulated upon aging (FIG. 5C).

We have previously shown that NR exerts beneficial effects on healthspan and lifespan, but whether it also improves proteostasis in mice is so far unknown. Treating old mice with NR led to a marked reduction of A11- and 4G8 positive-amyloid-like deposits in both TA and forelimbs muscle tissues (FIGS. 5A-5C), while partially restoring the levels of SDHB and MTCO1 in aged animals and increasing the expression of the MSR signature genes (FIG. 5C and FIGS. 12E-12F). To test the effects of NR in an established mouse model of Aβ amyloidosis, we also treated APP/PSEN1 AD mice with NR and assessed the levels of Aβ plaques in brain with Thioflavin S. NR administration robustly reduced Aβ deposits in cortex tissues of the AD mice (FIG. 5D), and similarly to what seen in the aged mice, induced the MSR signature and Oxphos protein levels (FIGS. 5E-5F). We hence propose that restoring or boosting mitochondrial function and proteostasis induces a conserved repair mechanism, from worm to mouse, that leads to decreased Aβ proteotoxicity.

Discussion

Proteotoxic stress in Aβ diseases, such as AD and IBM, is associated with mitochondrial dysfunction, and reduced Oxphos activity has been considered one of the major hallmarks of these diseases. Here, we identify a cross-species mitochondrial stress response signature that implicates mitochondrial proteostasis as a key mechanism in the response to Aβ proteotoxic stress. Most importantly, we show that Aβ accumulation induces both UPR^{mt} and mitophagy in a strikingly conserved manner from C. elegans to humans. Based on our results, we speculate that it must involve the alteration of a basic, conserved functional process, such as for instance mitochondrial import, which is linked to the activation of the UPR^{mt}, is perturbed during Aβ proteotoxic stress and is downregulated in AD patients in our analyses. Our work also provides solid evidence that mitochondria play an active role in the pathogenesis of Aβ diseases, as reducing mitochondrial homeostasis via atfs-1 depletion in GMC101 worms aggravates the hallmarks of the disease (FIG. 12G); conversely, boosting mitochondrial proteostasis by increasing the UPR^{mt} and mitophagy decreases protein aggregation, restores worm fitness and delays disease progression, ultimately translating in increased lifespan. Similarly, in mammals we showed that dox and NR can decrease Aβ accumulation in a neuronal cell model and that NR treatment reduces amyloid plaque formation in brain tissues of APP/PSEN1 AD mice, and amyloid-like aggregates in muscles of aged C57BL/6J mice. These findings indicate that treatments aimed at boosting mitochondrial function and proteostasis may decrease the formation of detrimental protein aggregates in the context of both proteotoxic disease and natural aging, both typified by reduced mitochondrial activity and loss of proteostasis. Furthermore, in light of our findings we would like to emphasize the potential value of the utilization of aged animal models as “natural” models of proteotoxic diseases. Together with initial evidence suggesting potential benefits of targeting dysfunctional mitochondria in AD and in view of recent findings linking mitochondrial stress to the induction of cytosolic proteostasis mechanisms, our data support the concept that enhancing mitochondrial proteostasis may hold promise to manage pervasive Aβ proteopathies, such as AD or IBM.

Methods

Animal Experiments

3×Tg AD mice, bearing human mutant APP_{Swe}, PS1M146V, and TauP301L transgenes, and wild-type, hybrid 129/C57BL6 mouse littermates were transcardially perfused with saline at 6 and 9 months of age (n=6/group) and brains from each group were hemisected. One hemisphere was immersion-fixed in 4% paraformaldehyde/0.1% glutaraldehyde for 24 hours and stored in cryoprotectant. From the other hemisphere, hippocampus, frontoparietal cortex, and cerebellum were rapidly dissected and snap-frozen.

Young (1 month old) and aged (20-24 months old) C57BL/6JRj mice were purchased from Janvier Labs. APP/PSEN1 mice (Tg(APP_{Swe},PSEN1dE9)85Dbo/Mmjax) were purchased from JAX. C57BL/6JR mice were fed with pellets containing vehicle or NR (400 mg/kg/day) for 6-8 weeks, while APP/PSEN1 mice were fed NR pellets for 10 weeks, starting at the age of 4 months. The pellets were prepared by mixing powdered chow diet (2016S, Harlan Laboratories) with water or with NR dissolved in water. Pellets were dried under a laminar flow hood for 48 hours. Mice were housed by groups of 2 to 5 animals per cage and randomized to 7-8 animals per experimental group according to their body weight. No blinding was used during the experiment procedures.

Ethical Approval

The experiments with postmortem human samples were authorized by the Michigan State University (MSU) Human Research Protection Program. The experiments with the 3×Tg mice were authorized by the MSU Institutional Review Board and Institutional Animal Care and Use Committee. The experiments with C57BL/6JRj and APP/PSEN1 mice were authorized by the local animal experimentation committee of the Canton de Vaud under licenses 2890 and 3207.

Human Brain Samples

Superior temporal cortex (Brodmann area 22) samples were obtained postmortem from participants in the Religious Orders Study who died with an antemortem clinical diagnosis of no cognitive impairment (NCI), mild cognitive impairment (MCI), or AD (n=8/group). Neuropsychological and clinical examinations, as well as postmortem diagnostic evaluations, have been described elsewhere. Demographic, antemortem cognitive testing, and postmortem diagnostic variables were compared among the groups using the nonparametric Kruskal-Wallis Test with Bonferroni correction for multiple comparisons. Gender and apoE ε4 allele distribution were compared using Fisher's Exact Test with Bonferroni correction.

Bioinformatics Analysis

For the in silico analysis of human brain expression datasets, we have used two sets of publicly available RNA-seq data: (1) from the Harvard Brain Tissue Resource Center (HBTRC), for human primary visual cortex (GN Accession: GN327) and human prefrontal cortex (GN Accession: GN328), and (2) from the Translational Genomics Research Institute, for the whole brain (GN Accession: GN314). These two datasets are publicly available on GeneNetwork (www.genenetwork.org). For the GSEA analysis of IBM studies, we used muscle transcript expression datasets from biopsies of healthy subjects and IBM patients (GSE3112, GSE39454). For correlation analysis, Pearson's r genetic correlation of the UPR^{mt}, mitophagy, ER stress and HSR gene sets was performed to establish the correlation between these pathways and genes that are associated or are causal to the development of AD. Analyses were performed using the hallmarks and canonical pathways gene sets databases.

Gene Expression Analyses

C. elegans:

A total of z3000 worms per condition, divided in 3 biological replicates, was recovered in M9 buffer from NGM plates and lysed in the TriPure RNA reagent. Each experiment was repeated twice. Total RNA was transcribed to cDNA using QuantiTect Reverse Transcription Kit (Qiagen). Expression of selected genes was analyzed using the LightCycler480 system (Roche) and LightCycler® 480 SYBR Green I Master reagent (Roche). For C. elegans, 2 housekeeping genes were used to normalize the expression data, namely actin (act-1) and peroxisomal membrane protein 3 (pmp-3).

Mouse:

Total RNA was extracted from tissues using TriPure RNA isolation reagent (Roche) according to the product manual. Expression of selected genes was analyzed using the LightCycler480 system (Roche) and LightCycler® 480 SYBR Green I Master reagent (Roche). The beta-2-Microglobulin B2m gene was used as housekeeping reference.

Human:

Total RNA was extracted using guanidine-isothiocyanate lysis (PureLink, Ambion, Waltham, Mass.) from cortex samples, and RNA integrity and concentration was verified using Bioanalysis (Agilent, Santa Clara, Calif.). Samples were randomized based on diagnostic group and assayed in triplicate on a real-time PCR cycler (ABI 7500, Applied Biosystems, Foster City) in 96-well optical plates. qPCR was performed using Taqman hydrolysis probe primer sets (Applied Biosystems) specific for the following human transcripts: HSPA9, HSPD1, YME1L1, DNM1L, BECN1, SQSTM1, PARK2, COX5A, CYC1. A primer set specific for human GAPDH was used as a control housekeeping transcript. For the APP_{Swe}-expressing SH-SY5Y cell line, total RNA was extracted from tissues using TriPure RNA isolation reagent, and expression of selected transcripts was analyzed using the LightCycler480 system (Roche) and LightCycler® 480 SYBR Green I Master reagent (Roche). The GAPDH gene was used as housekeeping reference. The ddCT method was employed to determine relative levels of each amplicon. Variance component analyses revealed relatively low levels of within-case variability, and the average value of the triplicate qPCR products from each case was used in subsequent analyses.

C. elegans Strains and Plasmids Generation, and RNAi Experiments

C. elegans strains were cultured at 20° C. on nematode growth media (NGM) agar plates seeded with E. coli strain OP50 unless stated otherwise. Strains used in this study were the wild-type Bristol N2, GMC101 [unc-54p::A-beta-1-42::unc-54 3′-UTR+mtl-2p::GFP], CL2122 [(pPD30.38) unc-54(vector)+(pCL26) mtl-2::GFP], CB4876 (clk-1(e2519)) and MQ1333 (nuo-6(qm200)). Strains were provided by the Caenorhabditis Genetics Center (University of Minnesota). The strain CL2122 was outcrossed 3 times in the N2 background, and subsequently used in the control experiments reported herein. AUW9 and AUW10: [GMC101+epfEx6[atfs-1p::atfs-1]], and AUW11: [CL2122+epfEx7[atfs-1p::atfs-1]] overexpression strains, and AUW12: [GMC101+clk-1(e2519) III], AUW13: [GMC101+nuo-6(qm200) I] were generated within this study.

atfs-1p::atfs-1 expression vector was created by amplifying 1488 bp sequence upstream from the transcription start site of atfs-1 coding region by using worm genomic DNA for the promoter region. The PCR product was digested with PciI and AgeI and ligated into the pPD30.38 expression vector containing gfp coding sequence cloned between inserted AgeI and NotI restriction sites. The atfs-1 coding sequence (CDS) was instead amplified using using C. elegans cDNA, and the PCR product was inserted into pPD30.38 downstream of the promoter region, between AgeI and NheI restriction sites. The correctness of the atfs-1p::atfs-1 construct was assessed by sequencing with the indicated pPD30.38 and atfs-1 seq. primers. Two independent GMC101 lines (AUW9 and AUW10), and one CL2122 strain (AUW11) carrying atfs-1p::atfs-1 transgene as extrachromosomal array were analyzed in the study. Injection marker myo-2p::gfp was cloned by amplifying 1179 bp sequence upstream from the transcription start site of myo-2 coding region by using C. elegans genomic DNA. The PCR product was digested with PciI and AgeI and ligated into the pPD30.38 expression vector containing atfs-1::gfp to replace atfs-1 promoter sequence between PciI and AgeI restriction sites. All transgenic strains were created by using microinjection. For the generation of the GMC101 lines AUW12 and AUW13, GMC101 males were generated after exposure of L4 worms to 30° C. for 3 h, and let mate with L4 from clk-1 or nuo-6 mutant strains. The derived progeny was selected for homozygosis of the GMC101 intestinal GFP marker for few generations, and the homozygosis of the clk-1 or nuo-6 mutant alleles verified by amplifying and sequencing a 500 bp region of the worms genomic DNA encompassing the desired mutation. Bacterial feeding RNAi experiments were carried out as described. Clones used were atfs-1 (ZC376.7), ubl-5 (F46F11.4), mrps-5 (E02A10.1), dct-1 (C14F5.1), daf-16 (R13H8.1), sir-2.1 (R11A8.4), xbp-1 (R74.3) and hsf-1 (Y53C10A.12). Clones were purchased from GeneService and verified by sequencing. The novel atfs-1 RNAi construct (atfs-1 #2) used in this study was generated by amplifying 1400 bp of the atfs-1 genomic DNA sequence, starting from the last exon of atfs-1. Gateway cloning (Thermo Scientfic) was used to insert the PCR product into the gateway vector pL4440gtwy (Addgene #11344), and verified by sequencing. atfs-1 knockdown was verified by qPCR for all the atfs-1 RNAi constructs used herein. For phenotypic studies (see below), worms at the L4 larval stage were allowed to reach adulthood and lay eggs on the treatment plates. The deriving F1 worms were shifted from 20° C. to 25° C. at the L4 stage to induce amyloid accumulation and paralysis, and phenotypes assessed over time as indicated. For double RNAi experiments, we used a combination of atfs-1 with mrps-5 RNAi constructs as indicated in the text, with 80% amount of atfs-1 and 20% mrps-5 RNAi. For mRNA analysis, synchronized L1 worms were exposed to the treatment plates, and shifted from 20° C. to 25° C. when reaching the L4 stage. After 24 hours incubation at 25° C., corresponding to day 1 of adulthood, they were harvested in M9 for analysis.

Pharmacological Treatment of C. elegans

Doxycycline was obtained from Sigma-Aldrich and dissolved in water. For experiments, a final concentration of 15 μg/mL was used. Olaparib (AZD2281) was dissolved in DMSO to experimental concentrations of 300 nM. Nicotinamide Riboside triflate (NR) was custom synthesized by Novalix (www.novalix-pharma.com/) and dissolved in water, and used at a final concentration of 1 mM. Compounds were added just before pouring the plates. For phenotyping experiments, parental F0 L4 worms were allowed to reach adulthood and lay eggs on the treatment plates. The deriving F1 worms were therefore exposed to compounds during the full life from eggs until death. For RNA analysis experiments, synchronized L1 worms were exposed to the compounds until harvest. To ensure a permanent exposure to the compound, plates were changed twice a week.

Worm Phenotypic Assays

Mobility:

C. elegans movement analysis was performed as described, starting from day 1 of adulthood, using the Movement Tracker software. The experiments were repeated at least twice.

Development:

50 adult worms per condition were transferred on NGM agar plates (10 worms per plate) and allowed to lay eggs for 3 h. Then they were removed and the number of eggs per plate was counted. 72 h later, the number of L1-L3 larvae, L4 and adult worms was counted. The experiment was done twice with five individual plates.

Paralysis and Death Score:

5 L4 worms per condition were allowed to reach adulthood and lay eggs on the treatment plates. 45 to 60 deriving F1 worms per condition were manually scored for paralysis after poking, as already described. Worms that were unable to respond to any, repeated stimulation, were scored as dead. Results are representative of the data obtained in at least three independent experiments.

Oxygen-Consumption Assays:

Oxygen consumption was measured using the Seahorse XF96 equipment (Seahorse Bioscience). Respiration rates were normalized to the number of worms in each individual well and calculated as averaged values of 5 to 6 repeated measurements. Each experiment was repeated at least twice.

MitoTracker® Orange CMTMRos Staining:

A population of 20 worms at L4 stage were transferred on plates containing MitoTracker® Orange CMTMRos (Thermo Scientific) at a final concentration of 2 ug/uL. The plates were incubated at 25° C. and the worms were collected and washed in 200 uL of M9 in order to remove the residual bacteria after 24 h of treatment. The worms were then incubated for 30 minutes on regular OP50 plates at 25° C. and mounted on an agar pad in M9 buffer for visualization. Mitochondria were observed by using confocal laser microscopy.

Phalloidin and DAPI Staining:

A population of 100 L4 worms was incubated for 24 h at 25° C. The worms were then washed in M9 and frozen in liquid nitrogen, immediately after they were lyophilized using a centrifugal evaporator. Worms were permeabilized using acetone. 2 U of phalloidin (Thermo Scientific) were resuspended in 20 uL of a buffer containing: Na-phosphate pH 7.5 (final concentration 0.2 mM), MgCl (final concentration 1 mM), SDS (final concentration 0.004%) and dH₂O to volume, and subsequently dispensed on NGM plates. The worms were incubated on the plates for 1 h in the dark and then washed 2 times in PBS and incubated in 20 uL of 2 ug/mL DAPI in PBS for 5 minutes. Following the immobilization, worms were observed by using confocal laser microscopy.

Quantitative Real-Time PCR for mtDNA/nuDNA Ratio

Absolute quantification of the mtDNA copy number in worms was performed by real-time PCR. Relative values for nd-1 and act-3 were compared within each sample to generate a ratio representing the relative level of mtDNA per nuclear genome. The results obtained were confirmed with a second mitochondrial gene MTCE.26. The average of at least three technical repeats was used for each biological data point. Each experiment was performed at least on five independent biological samples.

Cell Culture and Treatments

The SH-SY5Y neuroblastoma cell line expressing the APP Swedish K670N/M671L double mutation (APP_{Swe}) was a kind gift of Prof. Cedazo-Minguez (Karolinska Institute, Sweden). Cells were grown in DMEM/F-12, supplemented with 10% fetal bovine serum (FBS, Gibco), GlutaMAX (100×, Gibco) and penicillin/streptomycin (1×, Gibco). Cells were selected in 4 μg/mL Geneticin® Selective Antibiotic (G418 Sulfate, Sigma) and grown for three generations before experiments with cells plated and passaged at 4×10\hat{ }3 cells/ml and 60% confluence, respectively. Cells were cultured at 37° C. under a 5% CO₂ atmosphere and tested for mycoplasma using Mycoprobe (#CUL001B, R&D systems) following the manufacturer's instructions. Cells were treated with 10 μg/mL dox, NR 1 or 3 mM, ISRIB 0.5 μM (Sigma), as indicated for 24 hours before cell harvesting or fixation. For the immunostaining, cells were fixed with 1× Formal-Fixx (Thermo Scientific) for 15 min. After 15 min permeabilization with 0.1% Triton X-100, cells were blocked in PBS supplemented with 5% fetal bovine serum for 1 hour and immunostained overnight, at 4° C., with the anti-β-Amyloid 1-42 (1:100, Millipore AB5078P). The secondary antibody was coupled to the Alexa-488 fluorochrome (Thermo Scientific), and nuclei were stained with DAPI (Invitrogen, D1306). After washing in PBS, cell slides were mounted with Dako mounting medium (Dako, S3023) and examined with a Zeiss LSM 700 confocal microscope (Carl Zeiss MicroImaging) equipped with a Plan-Apochromat 40×/1.3 NA oil immersion objective lens using a 488 nm laser. Laser power was set at the lowest intensity allowing clear visualization of the signal. Imaging settings were maintained with the same parameters for comparison between different experimental conditions.

Western Blot Analysis

C. elegans:

Worms were lysed by sonication with RIPA buffer containing protease and phosphatase inhibitors (Roche), and analyzed by SDS-PAGE and western blot. The concentration of extracted protein was determined by using the Bio-Rad Protein Assay. Proteins were detected using the following antibodies: anti-β-actin (Sigma), anti-tubulin (Santa Cruz), atp-5, ucr-1 (Oxphos cocktail, Abcam), anti-β-Amyloid, 1-16 (6E10) (BioLegend). In addition to the housekeeping proteins, loading was monitored by Ponceau Red to ensure a homogeneous loading. Pixel intensity was quantified by using ImageJ software. Each immunoblot experiment was repeated at least twice using 3 biological replicates each containing approximately 1000 worms.

Mouse:

Frozen cortex tissue samples were lysed by mechanical homogenization with RIPA buffer containing protease and phosphatase inhibitors, and analyzed by SDS-PAGE and western blot. Subsequently, the concentration of extracted protein was determined by using the Bio-Rad Protein Assay. Proteins were detected using the following antibodies: HSP60 (Enzo Life Science), CLPP (Sigma), anti-GAPDH (14C10) (Cell Signaling), LONP1(Sigma), PINK1 (Novus Biologicals), LC3 A/B (Cell Signaling), SDHB (Oxphos cocktail, Abcam), MTCO1 (Abcam), Ubiquitin (Enzo), P62 (BD Transduction Laboratories), Phopsho P62 (Cell Signaling), VDAC (Abcam), P3-Amyloid, 17-24 (4G8) (BioLegend). In addition to the housekeeping proteins, loading was monitored by Ponceau Red to ensure a homogeneous loading. Antibody detection reactions for all the immunoblot experiments were developed by enhanced chemiluminescence (Advansta) and imaged using the c300 imaging system (Azure Biosystems). Pixel intensity was quantified by using ImageJ software.

Human:

Frozen cortex tissue samples were prepared as previously described. Samples were randomized based on diagnostic group and assayed in triplicate. For CLPP, blots were incubated overnight at 4° C. with a mouse monoclonal antibody to CLPP (1:1000; clone 2E1D9, ProteinTech) and then incubated for one hour with near-infrared-labeled goat anti-mouse IgG secondary antiserum (IRDye 800LT, 1:10,000; Licor) and analyzed on an Odyssey imaging system (Licor). Following imaging, the membranes were stripped and re-probed with a mouse monoclonal GAPDH antibody (1:10′000; clone 2D9, Origene) overnight followed by 1-hour incubation with near-infrared-labeled goat anti-mouse IgG secondary antiserum and Odyssey imaging. For mtDnaJ/Tid1, blots were incubated overnight at 4° C. with both a mouse monoclonal antibody to mtDnaJ/Tid1 (1:500; clone RS13, Cell Signaling) and the GAPDH antibody, followed by goat anti-mouse IgG incubation and Odyssey imaging. Signals for CLPP and mtDnaJ were normalized to GAPDH for quantitative analysis.

Citrate Synthase Activity Assay

Citrate synthase (CS) enzymatic activity was determined in mouse cortex samples and C. elegans using the CS assay kit (Sigma). Absorbance at 412 nm was recorded on a Victor X4 (PerkinElmer) with 10 readings over the 1.5 min timespan. These readings were in the linear range of enzymatic activity. The difference between baseline and oxaloacetate-treated samples was obtained and used to calculate total citrate synthase activity according to the formula provided in the manual. The obtained values were normalized by the amount of protein used for each sample.

Histology

TA muscles were harvested from anaesthetized mice and immediately frozen in Tissue-TEK® OCT compound (PST). 8-μm cryosections were collected and fixed with 4% paraformaldehyde. For immunostainings, heat activated antigen retrieval was performed in pH 6.0 citrate buffer for 10 min at 95° C. After washing with PBS-0.1% tween 20 (PBST), the sections were blocked with 10% affinipure Fab goat anti mouse IgG (Jackson Immunoresearch) in PBST for 60 min and PBST containing 2% BSA and 5% goat serum for 30 min at room temperature. Primary antibodies were then applied over night at 4° C. The following antibodies were used: anti-Oligomer A11 (Thermo Scientific, AHB0052), purified anti-β-Amyloid (4G8) (Biolegend, 800701). Subsequently, the slides were washed in PBST and incubated with appropriate secondary antibodies and labeling dyes. For immunofluorescence, secondary antibodies were coupled to Alexa-488 or Alexa-568 fluorochromes (Life technology), and nuclei were stained with DAPI (Invitrogen, D1306). After washing in PBST, tissue sections were mounted with Dako mounting medium (Dako, S3023). Images were acquired using Leica DMI 4000 (Leica Microsystems) or Olympus Slide Scanner VS120 (Olympus) at the same exposure time.

Brains hemispheres were harvested from anaesthetized mice and immediately frozen in isopentane. 8-μm cryosections were collected and fixed with 4% paraformaldehyde. For immunostainings, sections were stained with 0.01% Thioflavin S (Sigma) for 15 min at room temperature, and after washes in ethanol and PBS, stained with Hoechst (Life Technology). After washing in PBS, tissue sections were mounted with Dako mounting medium. Images were acquired using Leica DM 5500 (Leica Microsystems) CMOS camera 2900 Color at the same exposure time. Quantitative analysis of the immunofluorescence data was carried out by histogram analysis of the fluorescence intensity at each pixel across the images using Image J (Fiji; National Institutes of Health). Appropriate thresholding was employed to all the images of each single experiment to eliminate background signal in the images before histogram analysis. Fluorescence intensity and signal positive areas were calculated using the integrated “analyse particles” tool of the Fiji software, and statistical analysis were performed using Prism 6 (GraphPad Software).

Tandem LC-MS/MS and Multiple Reaction Monitoring Mass Spectrometry Analysis

A detailed description of the analytical approach used for IP-MS as well as LC-MS/MS parameters used for targeted mass spectrometry and MRM is provided in the supplementary method section. A complete list of all identified proteins from the IP-4G8 pull-down is not provided herein.

Statistical Analyses

Differences between two groups were assessed using two-tailed t tests. Differences between more than two groups were assessed by using one-way ANOVA. To compare the interaction between two factors, two-way ANOVA tests were performed. Analysis of variance, assessed by Bonferroni's multiple-comparison test, was used when comparing more than two groups. GraphPad Prism 6 (GraphPad Software, Inc.) was used for all statistical analyses. Variability in all plots and graphs is represented as the SEM. All P values<0.05 were considered to be significant. *P<0.05; **P≤0.01; ***P≤0.001; ****P≤0.0001 instead stated otherwise. All animal experiments were performed once. Animals that showed signs of severity, predefined by the animal authorizations were euthanized. These animals, together with those who died spontaneously during the experiments, were excluded from the calculations. These criteria were established before starting the experiments. For motility, fitness and death scoring experiments in C. elegans, sample size was estimated based on the known variability of the assay. All experiments were done non-blinded and repeated at least twice.

### REFERENCES FOR EXAMPLE 1

1 Alzheimer's, A. 2016 Alzheimer's disease facts and figures. Alzheimer's & dementia: the journal of the Alzheimer's Association 12, 459-509, (2016).
2 Mukherjee, A., Morales-Scheihing, D., Butler, P. C. & Soto, C. Type 2 diabetes as a protein misfolding disease. Trends in molecular medicine 21, 439-449, (2015).
3 Dember, L. M. Amyloidosis-associated kidney disease. Journal of the American Society of Nephrology: JASN 17, 3458-3471, (2006).
4 Askanas, V. & Engel, W. K. Sporadic inclusion-body myositis: conformational multifactorial ageing-related degenerative muscle disease associated with proteasomal and lysosomal inhibition, endoplasmic reticulum stress, and accumulation of amyloid-beta42 oligomers and phosphorylated tau. Presse medicale 40, e219-235, (2011).
5 Ahmed, M. et al. Targeting protein homeostasis in sporadic inclusion body myositis. Science translational medicine 8, 331ra341, (2016).
6 Gauthier, S. et al. Why has therapy development for dementia failed in the last two decades? Alzheimer's & dementia: the journal of the Alzheimer's Association 12, 60-64, (2016).
7 Mastaglia, F. L. & Needham, M. Inclusion body myositis: a review of clinical and genetic aspects, diagnostic criteria and therapeutic approaches. Journal of clinical neuroscience: official journal of the Neurosurgical Society of Australasia 22, 6-13, (2015).
8 Soejitno, A., Tjan, A. & Purwata, T. E. Alzheimer's Disease: Lessons Learned from Amyloidocentric Clinical Trials. CNS drugs 29, 487-502, (2015).
9 Herrup, K. et al. Beyond amyloid: getting real about nonamyloid targets in Alzheimer's disease. Alzheimer's & dementia: the journal of the Alzheimer's Association 9, 452-458 e451, (2013).
10 Selfridge, J. E., E, L., Lu, J. & Swerdlow, R. H. Role of mitochondrial homeostasis and dynamics in Alzheimer's disease. Neurobiology of disease 51, 3-12, (2013).
11 Joshi, P. R. et al. Functional relevance of mitochondrial abnormalities in sporadic inclusion body myositis. Journal of clinical neuroscience: official journal of the Neurosurgical Society of Australasia 21, 1959-1963, (2014).
12 Rygiel, K. A. et al. Mitochondrial and inflammatory changes in sporadic inclusion body myositis. Neuropathology and applied neurobiology 41, 288-303, (2015).
13 Pellegrino, M. W. & Haynes, C. M. Mitophagy and the mitochondrial unfolded protein response in neurodegeneration and bacterial infection. BMC biology 13, 22, (2015).
14 Greenberg, S. A. et al. Plasma cells in muscle in inclusion body myositis and polymyositis. Neurology 65, 1782-1787, (2005).
15 Zhu, W. et al. Genomic signatures characterize leukocyte infiltration in myositis muscles. BMC medical genomics 5, 53, (2012).
16 Beck, J. S., Mufson, E. J. & Counts, S. E. Evidence for Mitochondrial UPR Gene Activation in Familial and Sporadic Alzheimer's Disease. Current Alzheimer research 13, 610-614, (2016).
17 Mufson, E. J. et al. Mild cognitive impairment: pathology and mechanisms. Acta neuropathologica 123, 13-30, (2012).
18 Oddo, S. et al. Triple-transgenic model of Alzheimer's disease with plaques and tangles: intracellular Abeta and synaptic dysfunction. Neuron 39, 409-421, (2003).
19 Jovaisaite, V., Mouchiroud, L. & Auwerx, J. The mitochondrial unfolded protein response, a conserved stress response pathway with implications in health and disease. The Journal of experimental biology 217, 137-143, (2014).
20 Cohen, E., Bieschke, J., Perciavalle, R. M., Kelly, J. W. & Dillin, A. Opposing activities protect against age-onset proteotoxicity. Science 313, 1604-1610, (2006).
21 Brehme, M. et al. A chaperome subnetwork safeguards proteostasis in aging and neurodegenerative disease. Cell reports 9, 1135-1150, (2014).
22 Link, C. D. C. elegans models of age-associated neurodegenerative diseases: lessons from transgenic worm models of Alzheimer's disease. Experimental gerontology 41, 1007-1013, (2006).
23 McColl, G. et al. Utility of an improved model of amyloid-beta (Abeta(1)(−)(4)(2)) toxicity in Caenorhabditis elegans for drug screening for Alzheimer's disease. Mol Neurodegener 7, 57, (2012).
24 Mouchiroud, L. et al. The Movement Tracker: A Flexible System for Automated Movement Analysis in Invertebrate Model Organisms. Current protocols in neuroscience 77, 8 37 31-38 37 21, (2016).
25 Nargund, A. M., Fiorese, C. J., Pellegrino, M. W., Deng, P. & Haynes, C. M. Mitochondrial and nuclear accumulation of the transcription factor ATFS-1 promotes OXPHOS recovery during the UPR(mt). Molecular cell 58, 123-133, (2015).
26 Nargund, A. M., Pellegrino, M. W., Fiorese, C. J., Baker, B. M. & Haynes, C. M. Mitochondrial import efficiency of ATFS-1 regulates mitochondrial UPR activation. Science 337, 587-590, (2012).
27 Prahlad, V. & Morimoto, R. I. Integrating the stress response: lessons for neurodegenerative diseases from C. elegans. Trends in cell biology 19, 52-61, (2009).
28 Houtkooper, R. H. et al. Mitonuclear protein imbalance as a conserved longevity mechanism. Nature 497, 451-457, (2013). 29 Kim, H. E. et al. Lipid Biosynthesis Coordinates a Mitochondrial-to-Cytosolic Stress Response. Cell 166, 1539-1552 e1516, (2016).
30 Benedetti, C., Haynes, C. M., Yang, Y., Harding, H. P. & Ron, D. Ubiquitin-like protein 5 positively regulates chaperone gene expression in the mitochondrial unfolded protein response. Genetics 174, 229-239, (2006).
31 Haynes, C. M., Petrova, K., Benedetti, C., Yang, Y. & Ron, D. ClpP mediates activation of a mitochondrial unfolded protein response in C. elegans. Developmental cell 13, 467-480, (2007).
32 Wong, A., Boutis, P. & Hekimi, S. Mutations in the clk-1 gene of Caenorhabditis elegans affect developmental and behavioral timing. Genetics 139, 1247-1259, (1995).
33 Yang, W. & Hekimi, S. Two modes of mitochondrial dysfunction lead independently to lifespan extension in Caenorhabditis elegans. Aging cell 9, 433-447, (2010).
34 Moullan, N. et al. Tetracyclines Disturb Mitochondrial Function across Eukaryotic Models: A Call for Caution in Biomedical Research. Cell reports, (2015).
35 Zheng, L. et al. Macroautophagy-generated increase of lysosomal amyloid beta-protein mediates oxidant-induced apoptosis of cultured neuroblastoma cells. Autophagy 7, 1528-1545, (2011).
36 Forloni, G., Colombo, L., Girola, L., Tagliavini, F. & Salmona, M. Anti-amyloidogenic activity of tetracyclines: studies in vitro. FEBS letters 487, 404-407, (2001).
37 Costa, R., Speretta, E., Crowther, D. C. & Cardoso, I. Testing the therapeutic potential of doxycycline in a Drosophila melanogaster model of Alzheimer disease. The Journal of biological chemistry 286, 41647-41655, (2011).
38 Loeb, M. B. et al. A randomized, controlled trial of doxycycline and rifampin for patients with Alzheimer's disease. Journal of the American Geriatrics Society 52, 381-387, (2004).
39 Quiros, P. M. et al. Multi-omics analysis identifies ATF4 as a key regulator of the mitochondrial stress response in mammals. The Journal of cell biology 216, 2027-2045, (2017).
40 Munch, C. & Harper, J. W. Mitochondrial unfolded protein response controls matrix pre-RNA processing and translation. Nature 534, 710-713, (2016).
41 Bao, X. R. et al. Mitochondrial dysfunction remodels one-carbon metabolism in human cells. eLife 5, (2016).
42 Sidrauski, C., McGeachy, A. M., Ingolia, N. T. & Walter, P. The small molecule ISRIB reverses the effects of elF2alpha phosphorylation on translation and stress granule assembly. eLife 4, (2015).
43 Palikaras, K., Lionaki, E. & Tavernarakis, N. Coordination of mitophagy and mitochondrial biogenesis during ageing in C. elegans. Nature 521, 525-528, (2015).
44 Mouchiroud, L. et al. The NAD(+)/sirtuin pathway modulates longevity through activation of mitochondrial UPR and FOXO signaling. Cell 154, 430-441, (2013).
45 Gariani, K. et al. Eliciting the mitochondrial unfolded protein response by nicotinamide adenine dinucleotide repletion reverses fatty liver disease in mice. Hepatology 63, 1190-1204, (2016).
46 Zhang, H. et al. NAD(+) repletion improves mitochondrial and stem cell function and enhances life span in mice. Science 352, 1436-1443, (2016).
47 Fang, E. F. et al. NAD+ Replenishment Improves Lifespan and Healthspan in Ataxia Telangiectasia Models via Mitophagy and DNA Repair. Cell metabolism 24, 566-581, (2016).
48 Riera, C. E., Merkwirth, C., De Magalhaes Filho, C. D. & Dillin, A. Signaling Networks Determining Life Span. Annual review of biochemistry 85, 35-64, (2016).
49 Labbadia, J. & Morimoto, R. I. The biology of proteostasis in aging and disease. Annual review of biochemistry 84, 435-464, (2015).
50 Rudinskiy, N. et al. Amyloid-beta oligomerization is associated with the generation of a typical peptide fragment fingerprint. Alzheimer's & dementia: the journal of the Alzheimer's Association 12, 996-1013, (2016).
51 Cenini, G., Rub, C., Bruderek, M. & Voos, W. Amyloid beta-peptides interfere with mitochondrial preprotein import competence by a coaggregation process. Molecular biology of the cell 27, 3257-3272, (2016).
52 Gong, B. et al. Nicotinamide riboside restores cognition through an upregulation of proliferator-activated receptor-gamma coactivator 1 alpha regulated beta-secretase 1 degradation and mitochondrial gene expression in Alzheimer's mouse models. Neurobiology of aging 34, 1581-1588, (2013).
53 Martire, S. et al. Bioenergetic Impairment in Animal and Cellular Models of Alzheimer's Disease: PARP-1 Inhibition Rescues Metabolic Dysfunctions. Journal of Alzheimer's disease: JAD 54, 307-324, (2016).
54 Wang, X. & Chen, X. J. A cytosolic network suppressing mitochondria-mediated proteostatic stress and cell death. Nature 524, 481-484, (2015).
55 Wrobel, L. et al. Mistargeted mitochondrial proteins activate a proteostatic response in the cytosol. Nature 524, 485-488, (2015).
56 D'Amico, D., Sorrentino, V. & Auwerx, J. Cytosolic Proteostasis Networks of the Mitochondrial Stress Response. Trends in biochemical sciences, (2017).
57 Tiernan, C. T. et al. Protein homeostasis gene dysregulation in pretangle-bearing nucleus basalis neurons during the progression of Alzheimer's disease. Neurobiology of aging 42, 80-90, (2016).
58 Liang, W. S. et al. Alzheimer's disease is associated with reduced expression of energy metabolism genes in posterior cingulate neurons. Proceedings of the National Academy of Sciences of the United States of America 105, 4441-4446, (2008).
59 Fonte, V. et al. Suppression of in vivo beta-amyloid peptide toxicity by overexpression of the HSP-16.2 small chaperone protein. The Journal of biological chemistry 283, 784-791, (2008).
60 Kamath, R. S., Martinez-Campos, M., Zipperlen, P., Fraser, A. G. & Ahringer, J. Effectiveness of specific RNA-mediated interference through ingested double-stranded RNA in Caenorhabditis elegans. Genome biology 2, RESEARCH0002, (2001).
61 Florez-McClure, M. L., Hohsfield, L. A., Fonte, G., Bealor, M. T. & Link, C. D. Decreased insulin-receptor signaling promotes the autophagic degradation of beta-amyloid peptide in C. elegans. Autophagy 3, 569-580, (2007).
62 Koopman, M. et al. A screening-based platform for the assessment of cellular respiration in Caenorhabditis elegans. Nature protocols 11, 1798-1816, (2016).
63 Ryu, D. et al. Urolithin A induces mitophagy and prolongs lifespan in C. elegans and increases muscle function in rodents. Nat Med 22, 879-888, (2016).
64 Counts, S. E., Nadeem, M., Lad, S. P., Wuu, J. & Mufson, E. J. Differential expression of synaptic proteins in the frontal and temporal cortex of elderly subjects with mild cognitive impairment. Journal of neuropathology and experimental neurology 65, 592-601, (2006).

### Example 2

Inhibition of the ceramide and sphingolipid biosynthesis pathway improves fitness in the GMC101 worms.

C. elegans strains were cultured at 20° C. on nematode growth media (NGM) agar plates seeded with E. coli strain HT115. The GMC101 strain [unc-54p::A-beta-1-42::unc-54 3′-1054 UTR+mtl-2p::GFP] (McColl et al., 2012) was provided by the Caenorhabditis Genetics Center (University of Minnesota). GMC101 worms constantly express the human Aβ isoform 1-42 in muscle cells, but adults only develop age-progressive paralysis and amyloid deposition in the body wall muscle after a temperature shift from 20 to 25° C. Given the muscle-targeted overexpression of the Aβ peptide in GMC101, this strain can be considered as a general model of Aβ disease, and equally suitable to mimic the proteotoxic phenotypes observed in Alzheimer's disease (AD) and inclusion body myositis (IBM).

In this assay, GMC101 worms were treated with Myriocin ((E,2S,3R,4R)-2-amino-3,4-dihydroxy-2-(hydroxymethyl)-14-oxoicos-6-enoic acid). Myriocin, also known as ISP-1/thermozymocidin, is an antibiotic derived from certain thermophilic fungi, including Mycelia sterilia (Miyake et al., 1995). Myriocin was described as a natural compound whose structure is homologous to sphingosine, therefore it acts as a potent inhibitor of sphingolipid biosynthesis, including the synthesis of sphingosine and ceramide (Miyake et al., 1995). This group of bioactive molecules, including sphingolipids and ceramides, are involved in numerous cellular processes, ranging from proliferation and differentiation of the cells to inflammatory responses and cellular apoptosis, and their accumulation can be detrimental (Hanada, 2003). Silencing of genes involved in sphingolipid biosynthesis in C. elegans has been shown to induce a mitochondrial stress response as well as the heat shock response, which are key pathways whose induction can restore proteostasis in worms subjected to proteotoxic stress (Kim et al., 2016).

During the assays, animals were exposed to Myriocin (dissolved in DMSO) from the egg stage on plates seeded with live HT115 E. coli bacteria. Control plates seeded with the same bacteria were prepared with the corresponding concentrations of DMSO. The day of the experiment (day 1 of adulthood, 3 days after hatching), the GMC101 population was shifted to 25° C. in order to induce the amyloid deposition in the body wall muscle. ˜1000 worms were used for western blotting assays.

Worms were lysed by sonication with RIPA buffer containing protease and phosphatase inhibitors (Roche), and analyzed by western blot. The concentration of extracted protein was determined by the Bio-Rad Protein Assay. Proteins were detected using the following antibodies: anti-β-actin (Sigma) and anti-β-Amyloid (BioLegend). In addition to the housekeeping proteins, loading was monitored by Ponceau Red to ensure a homogeneous loading. The immunoblot experiment employed 3 biological replicates, each containing approximately 1000 worms.

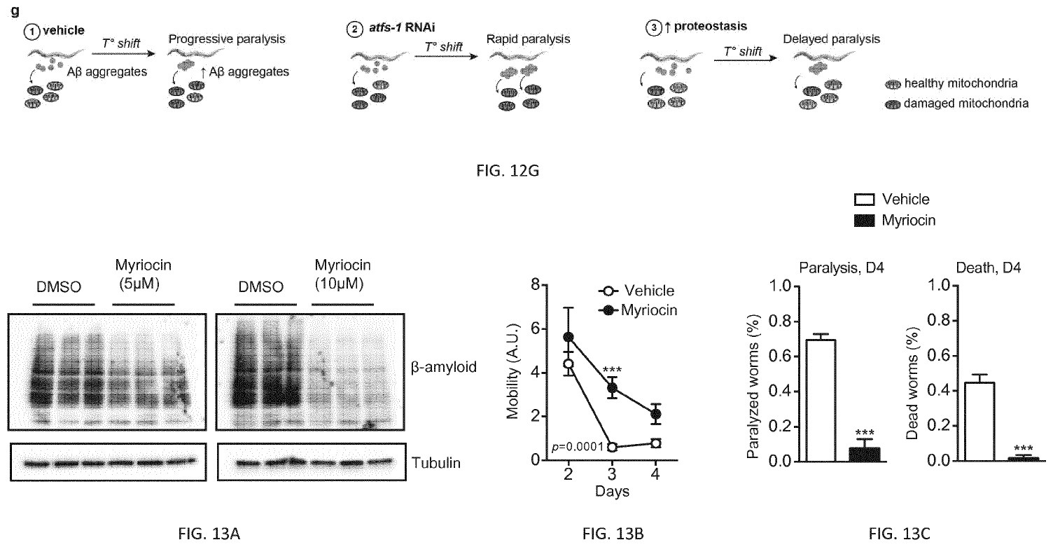

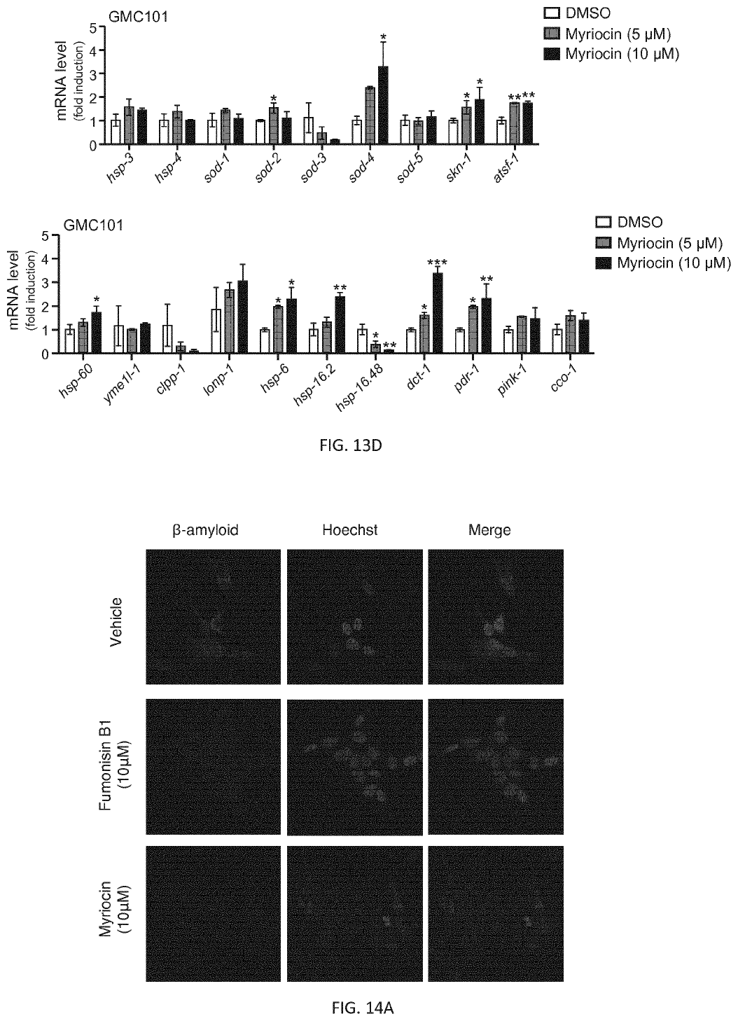

Treatment with two different doses of Myriocin, i.e. 5 uM and 10 uM, dose-dependently decreased Aβ amyloid deposition in treated GMC101 worms at Day 1 of adulthood (FIG. 13A).

### Example 3

Inhibition of the ceramide and sphingolipid biosynthesis pathway increases proteostasis in the GMC101 worms.

C. elegans movement analysis was performed as described (Mouchiroud et al., 2016), using the Movement Tracker software. ˜50 adult worms were used per condition for movement assays. Treatment with 10 uM Myriocin increased the mobility of the GMC101 worms within a period of 4 days (FIG. 13B).

To score for paralysis and death, 50 worms per condition were manually scored after poking as described (McCool et al., 2012; Florez-McClure et al., 2007). Worms that were unable to respond to repeated stimulation were scored as dead. Treatment with 10 uM Myriocin decreased both paralysis and death of the GMC101 worms after a period of 4 days (FIG. 13C).

### Example 4

Inhibition of the ceramide and sphingolipid biosynthesis pathway increases proteostasis in a human neuronal cell line model of Aβ amyloid aggregation.

The SH-SY5Y neuroblastoma cell line expresses the APP Swedish K670N/M671L double mutation (APP_{Swe}) (Zheng et al., 2011) and accumulates intracellular Aβ amyloid aggregates. Cells were grown in DMEM/F-12, supplemented with 10% fetal bovine serum (FBS, Gibco), GlutaMAX (100×, Gibco) and penicillin/streptomycin (1×, Gibco). Cells were selected in 4 μg/mL Geneticin® Selective Antibiotic (G418 Sulfate, Sigma) and grown for three generations before experiments with cells plated and passaged at 4×10\hat{ }3 cells/ml and 60% confluence, respectively. Cells were cultured at 37° C. under a 5% CO₂ atmosphere and tested for mycoplasma using Mycoprobe (#CUL001B, R&D systems), following the manufacturer's instructions. Myriocin was dissolved in DMSO.

Fumonisin B₁ ((2S,2'S)-2,2′-{[(5S,6R,7R,9R,11S,16R,18S,19S)-19-Amino-11,16,18-trihydroxy-5,9-dimethylicosane-6,7-diyl]bis[oxy(2-oxoethane-2,1-diyl)]}disuccinic acid) is the most prevalent member of a family of toxins, known as fumonisins, produced by several species of Fusarium molds, which occur mainly in maize (corn), wheat and other cereals.

Myriocin and Fumonisin B₁ are natural compounds whose structure is homologous to sphingosine, therefore they act as potent inhibitors of sphingolipids biosynthesis, including the synthesis of sphingosine and ceramide (Miyake et al., 1995, Wang et al., 1991).

Cells were treated with 10 uM Myriocin and Fumonisin B₁ for 24 hours before harvesting or fixation (FIG. 3A). For immunostaining, cells were fixed with 1× Formal-Fixx (Thermo Scientific) for 15 min. After 15 min permeabilization with 0.1% Triton X-100, cells were blocked in PBS supplemented with 5% fetal bovine serum for 1 hour and immunostained overnight, at 4° C., with the anti-β-Amyloid (6E10) antibody (1:100, BioLegend, 803002). The secondary antibody was coupled to the Alexa-568 fluorochrome (Thermo Fisher), and nuclei were stained with DAPI (Invitrogen, D1306). After washing in PBS, cell slides were mounted with Dako mounting medium (Dako, S3023) and examined with a Zeiss LSM 700 confocal microscope (Carl Zeiss MicroImaging) equipped with a Plan-Apochromat 40×/1.3 NA oil immersion objective lens using a 555 nm laser. Laser power was set at the lowest intensity allowing clear visualization of the signal. Imaging settings were maintained with the same parameters for comparison between different experimental conditions.

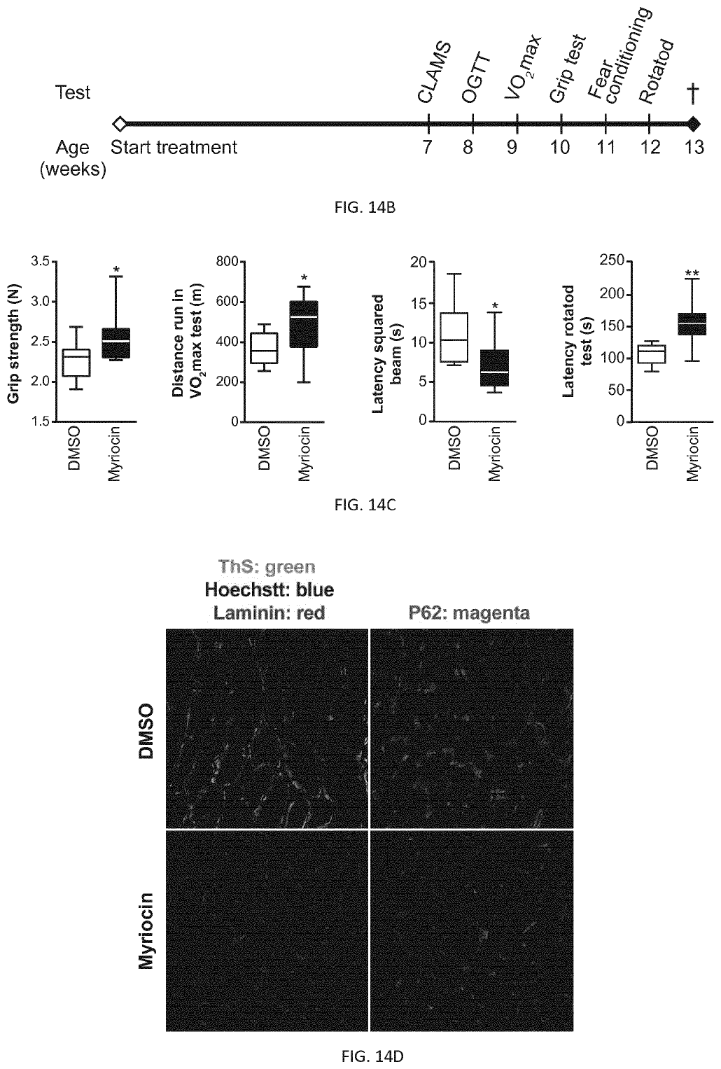

Treatment of human neuroblastoma cell line expressing the APP_{Swe} with Myriocin and Fumonisin B1, both at 10 uM, significantly reduced intracellular Aβ deposits (FIG. 14).

### REFERENCES FOR EXAMPLES 2-4

Florez-McClure, M. L. et al. Decreased insulin-receptor signaling promotes the autophagic degradation of beta-amyloid peptide in C. elegans. (2007). Autophagy 3, 569-580. Hanada, K. Serine palmitoyltransferase, a key enzyme of sphingolipid metabolism. (2003). Biochim Biophys Acta 1632, 16-30.
Kim, H. E. et al. Lipid biosynthesis coordinates a mitochondrial-to-cytosolic stress response. (2016). Cell 166, 1539-1552.
McColl, G. et al. Utility of an improved model of amyloid-beta (Abeta(1)(−)(4)(2)) toxicity in Caenorhabditis elegans for drug screening for Alzheimer's disease. (2012). Mol Neurodegener 7, 57.
Mouchiroud, L. et al. The Movement Tracker: A flexible system for automated movement analysis in invertebrate model organisms. (2016). Curr Protoc Neurosci 77, 8:37.1-8:37.21.
Miyake, Y. et al. Serine palmitoyltransferase is the primary target of a sphingosine-like immunosuppressant, ISP-1/myriocin. (1995). Biochem Biophys Res Commun 211, 396-403.
Wang, E. et al. Inhibition of sphingolipid biosynthesis by fumonisins. (1991). J. Biol. Chem. 266, 14486-14490.
Zheng, L. et al. Macroautophagy-generated increase of lysosomal amyloid beta protein mediates oxidant-induced apoptosis of cultured neuroblastoma cells. (2011). Autophagy 7, 1528-1545.

### Example 5

Mitophagy inducers, such as Urolithin A (UA), improve fitness in the GMC101 worms.

C. elegans strains were cultured at 20° C. on nematode growth media (NGM) agar plates seeded with E. coli strain HT115. The GMC101 strain [unc-54p::A-beta-1-42::unc-54 3′-1054 UTR+mtl-2p::GFP] (McColl et al., 2012) was provided by the Caenorhabditis Genetics Center (University of Minnesota). GMC101 worms constantly express the human Aβ isoform 1-42 in muscle cells, but adults only develop age-progressive paralysis and amyloid deposition in the body wall muscle after a temperature shift from 20 to 25° C. Given the muscle-targeted overexpression of the Aβ peptide in GMC101, this strain can be considered as a general model of Aβ disease, and equally suitable to mimic the proteotoxic phenotypes observed in Alzheimer's disease (AD) and inclusion body myositis (IBM).

Urolithin A (3,8-Dihydroxyurolithin) was dissolved in DMSO. Urolithin A (UA) is an ellagitannin- and ellagic acid-derived metabolite produced by mammalian colonic microflora, including human colonic microflora (Espin et al., 2013; Seeram et al., 2006). UA was described as a natural compound that induces mitophagy both in vitro and in vivo (Ryu et al., 2016). Mitophagy is the molecular process allowing the removal of damaged mitochondria through autophagy (Youle and Narendra, 2011). This process is critical for maintaining proper cellular functions, especially during aging when mitochondrial functions start to decline. In the nematode C. elegans, UA was shown to prevent the accumulation of dysfunctional mitochondria with age and extended lifespan through the activation of mitophagy (Ryu et al., 2016). Furthermore, C. elegans treated with UA maintained normal activity during aging, including mobility and pharyngeal pumping, while maintaining mitochondrial respiratory capacity. These effects are conserved in rodents, where UA significantly improved exercise capacity in mouse models of age-related decline of muscle function, as well as in young rats (Ryu et al., 2016).

During the assays, animals were exposed to the compound UA from egg stage on plates seeded with live HT115 E. coli bacteria. Control plates seeded with the same bacteria were prepared with the corresponding concentrations of DMSO (0.1%). The day of the experiment (day 1 of adulthood, 3 days after hatching), the GMC101 population was shifted to 25° C. in order to induce the amyloid deposition in the body wall muscle. ˜50 adult worms were used per conditions.

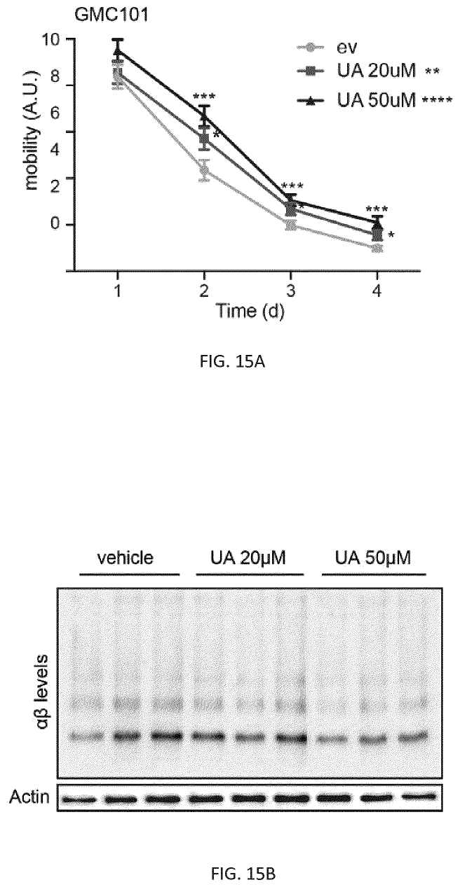

C. elegans movement analysis was performed as described in (Mouchiroud et al., 2016), using the Movement Tracker software within a period of 4 days. Treatment with two different doses of UA, ie 20 uM and 50 uM, dose-dependently increased the mobility of the GMC101 worms (FIG. 15A).

### REFERENCES FOR EXAMPLE 5

Espin, J. C. et al. Biological significance of urolithins, the gut microbial ellagic acid-derived metabolites: the evidence so far. (2013). Evid Based Complement Alternat Med 2013, 270418.
McColl, G. et al. Utility of an improved model of amyloid-beta (Abeta(1)(−)(4)(2)) toxicity in Caenorhabditis elegans for drug screening for Alzheimer's disease. (2012). Mol Neurodegener 7, 57.
Mouchiroud, L. et al. The Movement Tracker: A flexible system for automated movement analysis in invertebrate model organisms. (2016). Curr Protoc Neurosci 77, 8:37.1-8:37.21.
Ryu, D. et al. Urolithin A induces mitophagy and prolongs lifespan in C. elegans and increases muscle function in rodents. (2016). Nat Med 22, 879-888.
Seeram, N. P. et al. Pomegranate juice ellagitannin metabolites are present in human plasma and some persist in urine for up to 48 hours. (2006). J Nutr 136, 2481-2485.
Youle, R. J. & Narendra, D. P. Mechanisms of mitophagy. (2011). Nat Rev Mol Cell Biol 12, 9-14.

### Example 6

Urolithin A (UA) increases proteostasis in the GMC101 worms.

Worms were lysed by sonication with RIPA buffer containing protease and phosphatase inhibitors (Roche), and analyzed by western blot. The concentration of extracted proteins was determined by the Bio-Rad Protein Assay. Proteins were detected using the following antibodies: anti-β-actin (Sigma) and anti-β-Amyloid (BioLegend). In addition to the housekeeping proteins, loading was monitored by Ponceau Red to ensure a homogeneous loading. The immunoblot experiment employed 3 biologicals replicates, each containing approximately 1000 worms.

The GMC101 worms were recovered 24 h after being shifted at 25° C. The population was treated with UA at two different concentrations, ie 20 uM and 50 uM. Aβ aggregation was dose-dependently reduced upon UA treatment, as shown by immunobloting (FIG. 15B).

### EQUIVALENTS

While the present invention has been described in conjunction with the specific embodiments set forth above, many alternatives, modifications and other variations thereof will be apparent to those of ordinary skill in the art. All such alternatives, modifications and variations are intended to fall within the spirit and scope of the present invention.

What is claimed is:

1. A method of treating an amyloid-3 peptide disease in a subject in need thereof, the method comprising administering to the subject a therapeutically effective amount of a compound that enhances mitochondrial proteostasis.

2. The method of claim 1, wherein the compound induces mitochondrial unfolded protein response (UPR^{mt}), mitochondrial biogenesis, or mitophagy.

3. The method of claim 1, wherein the compound modulates lipid metabolism.

4. The method of claim 2, wherein the compound that induces UPR^{mt} is selected from the group consisting of tetracycline, chlortetracycline, oxytetracycline, demeclocycline, lymecycline, meclocycline, methacycline, minocycline, rolitetracycline, doxycycline, tigecycline, actinonin, chloramphenicol, and a compound that blocks mitochondrial import.

5. The method of claim 4, wherein doxycycline is administered at 90 mg/kg/day in food for 9-10 weeks or 200-500 mg/kg/day in water for about 9-10 weeks.

6. The method of claim 2, wherein the compound that induces mitochondrial biogenesis is selected from the group consisting of an NAD⁺ boosting compound, a PARP inhibitor, a CD38 or CD157/BST1 inhibitor, an activator of nicotinamide phosphoribosyltransferases (NAMPT), an inhibitor of nicotinamide N methyltransferases (NNMT), and an inhibitor of α-amino-β-carboxymuconate-ε-semialdehyde decarboxylase (ACMSD).

7. The method of claim 6, wherein the NAD⁺ boosting compound is an NAD⁺ precursor.

8. The method of claim 7, wherein the NAD⁺ precursor is nicotinamide riboside, nicotinamide mononucleotide, nicotinic acid, or nicotinamide.

9. The method of claim 8, wherein nicotinamide riboside is administered at 400 mg/kg/day in food for about 9-10 weeks.

10. The method of claim 6, wherein the PARP inhibitor is 3-aminobenzamide, olaparib, velaparib, rucaparib, iniparib, talazoparib, CEP-9722, E7016, or niraparib.

11. The method of claim 10, wherein olaparib or velaparib is administered at 300 mg/kg/day in food for about 9-10 weeks.

12. The method of claim 6, wherein the CD38 or CD157/BST1 inhibitor is GSK 897-78c, apigenin, or quercetin.

13. The method of claim 6, wherein the activator of NAMPT is PC73.

14. The method of claim 6, wherein the inhibitor of ACMSD is a phthalate ester or pyrazinamide.

15. The method of claim 2, wherein the compound that induces mitophagy is selected from the group consisting of Urolithin A, Urolithin B, 5-aminoimidazole-4-carboxamide-ribonucleoside, salicylate, A-769662, 1-(2,6-Dichlorophenyl)-6-[[4-(2-hydroxyethoxy)phenyl]methyl]-3-propan-2-yl-2H-pyrazolo[3,4-d]pyrimidin-4-one, and metformin.

16. The method of claim 3, wherein the compound that modulates lipid metabolism is selected from the group consisting of perhexiline, a fibrate, and a statin.

17. The method of claim 16, wherein the fibrate is fenofibrate, clofibrate, or bezafibrate.

18. The method of claim 16, wherein the statin is lovastatin, simvastatin, atorvastatin, or fluvastatin.

19. The method of claim 2, wherein the compound that modulates lipid metabolism also inhibits sphingosin and ceramide synthesis.

20. The method of claim 19, wherein the compound that modulates lipid metabolism is myriocin, fumonisin B1, amlodipine, astemizole, benztropine, bepridil, or doxepine.

21. The method of claim 20, wherein myriocin is administered at 0.4 mg/kg body weight.

22. The method of any one of claims 1-3, wherein the compound is a natural product.

23. The method of claim 22, wherein the natural product is Urolithin A, nicotinamide riboside, nicotinamide mononucleotide, nicotinic acid, nicotinamide, or quercetin.

24. The method of any one of the preceding claims, wherein the amyloid-3 peptide disease is a muscle disease.

25. The method of claim 24, wherein the muscle disease is inclusion body myositis, age-related sarcopenia, frailty of the elderly, or muscular dystrophy.

26. The method of claim 25, wherein the muscular dystrophy is Duchenne's or Becker's muscular dystrophy.

27. The method of any one of claims 1-23, wherein the amyloid-3 peptide disease is a metabolic disease.

28. The method of claim 27, wherein the metabolic disease is type 2 diabetes, an amyloid kidney disease, or an amyloid heart disease.

29. The method of any one of claims 1-23, wherein the amyloid-3 peptide disease is Alzheimer's disease, Dementia, Parkinson's disease, Huntington's disease, or amyotropic lateral sclerosis.

30. The method of any one of the preceding claims, wherein the subject is a human.

31. The method of any one of the preceding claims, comprising administering to the subject a therapeutically effective amount of at least two compounds, each of which enhances mitochondrial proteostasis.

32. The method of claim 31, wherein the at least two compounds are administered sequentially or simultaneously.


SEQUENCE LISTING (2 sequences)

SEQ ID NO: 1 - protein, 42 aa, Homo sapiens
DAEFGHDSGFEVRHQKLVFFAEDVGSNKGAIIGLMVGGVVIA

SEQ ID NO: 2 - protein, 12 aa, Artificial Sequence
  Artificial polypeptide
KLVFFAEDVGSN